(19) United States
(12) Patent Application Publication
(10) Pub. No.: US 2013/0189261 A1
(43) Pub. Date: Jul. 25, 2013

(54) TCR Complex Immunotherapeutics

(71) Applicant: Emergent Product Development Seattle, LLC, Seattle, WA (US)

(72) Inventors: Valerie ODEGARD, Seattle, WA (US); Catherine J. Mcmahan, Seattle, WA (US); Peter Robert Baum, Seattle, WA (US); Peter Armstrong Thompson, Bellevue, WA (US); Philip Tan, Edmonds, WA (US); John W. Blankenship, Seattle, WA (US); Sateesh Kumar Natarajan, Redmond, WA (US)

(73) Assignee: Emergent Product Development Seattle, LLC, Seattle, WA (US)

(21) Appl. No.: 13/830,959

(22) Filed: Mar. 14, 2013

Related U.S. Application Data
(63) Continuation of application No. 13/123,509, filed on May 27, 2011.
(60) Provisional application No. 61/104,608, filed on Oct. 10, 2008.
(60) Provisional application No. 61/148,341, filed on Jan. 29, 2009.

(51) Int. Cl. A61K 39/395; C07K 16/46

(52) U.S. Cl. CPC A61K 39/3955; C07K 16/46
USPC 424/135.1; 530/387.3; 536/23.4; 435/320.1

(57) ABSTRACT

Single chain fusion proteins that specifically bind to a TCR complex or a component thereof, such as TCRα, TCRβ, or CD3ε, along with compositions and methods of use thereof are provided.

The content of the electronically submitted sequence listing (Name: sequencelisting_ascii.txt, Size: 636,648 bytes; and Date of Creation: Mar. 14, 2013) is herein incorporated by reference in its entirety.

Related applications: U.S. patent application Ser. No. 13/123,509, which is the National Stage of International Application No. PCT/US2009/060286, filed Oct. 9, 2009, U.S. Provisional Patent Application No. 61/104,608, filed Oct. 10, 2008, and U.S. Provisional Patent Application No. 61/148,341, filed Jan. 29, 2009 are incorporated herein by reference in their entireties.

### BACKGROUND

1. Technical Field

The present disclosure relates to immunologically active, recombinant binding proteins and, in particular, to single chain fusion proteins specific for a TCR complex or component thereof, such as CD3. The present disclosure also relates to compositions and methods for treating autoimmune diseases and other disorders or conditions (e.g., transplant rejection).

2. Description of the Related Art

Targeting the TCR complex on human T cells with anti-CD3 monoclonal antibodies has long been used in the treatment of organ allograft rejection. Mouse monoclonal antibodies specific for human CD3, such as OKT3 (Kung et al. (1979) Science 206: 347-9), were the first generation of such treatments. Although OKT3 has strong immunosuppressive potency, its clinical use was hampered by serious side effects linked to its immunogenic and mitogenic potentials (Chatenoud (2003) Nature Reviews 3:123-132). It induced an anti-globulin response, promoting its own rapid clearance and neutralization (Chatenoud et al. (1982) Eur. J. Immunol. 137:830-8). In addition, OKT3 induced T-cell proliferation and cytokine production in vitro and led to a large scale release of cytokine in vivo (Hirsch et al. (1989) J. Immunol. 142: 737-43, 1989). The cytokine release (also referred to as “cytokine storm”) in turn led to a “flu-like” syndrome, characterized by fever, chills, headaches, nausea, vomiting, diarrhea, respiratory distress, septic meningitis and hypotension (Chatenoud, 2003). Such serious side effects limited the more widespread use of OKT3 in transplantation as well as the extension of its use to other clinical fields such as autoimmunity (Id.).

To reduce the side effects of the first generation of anti-CD3 monoclonal antibodies, a second generation of genetically engineered anti-CD3 monoclonal antibodies had been developed not only by grafting complementarity-determining regions (CDRs) of murine anti-CD3 monoclonal antibodies into human IgG sequences, but also by introducing non-FcR-binding mutations into the Fc (Cole et al. (1999) Transplantation 68: 563; Cole et al. (1997) J. Immunol. 159: 3613). Humanization of the murine monoclonal antibodies results in decreased immunogenicity and improved mAb half-life (Id.). In addition, non-FcR-binding mAbs have reduced potential for inducing cytokine release and acute toxicity in vivo (Chatenoud et al. (1989) N. Engl. J. Med. 320:1420). However, the cytokine release, even at a reduced level, is still dose-limiting and toxic at very low drug doses (micrograms/patient) (Plevy et al., (2007) Gastroenterology 133:1414-1422).

Several difficulties exist for improving anti-CD3/TCR-directed therapy. For example, the mechanism of immunosuppression mediated by anti-CD3 monoclonal antibodies is complex and not fully understood. It is believed that such antibodies function through four mechanisms: cell coating, cell depletion, TCR down-modulation and cell signaling, with the latter two as the main mechanisms (Chatenoud (2003) Nature Reviews:123-132). It is further believed that the induction of cytokine storm and in vivo T cell activation are required for efficacy of CD3/TCR-directed therapy (Carpenter et al. (2000) J. Immunology 165:6205-13). Finally, second generation anti-CD3 monoclonal antibodies reported to be “non-activating” in vitro have still induced a cytokine storm in vivo.

A number of anti-CD3 directed antibodies are currently being tested in the clinic for use in autoimmune disease, inflammatory disease, and transplant patient. These antibodies include hOKT3γ1(Ala-Ala) (Macrogenics), visilizumab (Nuvion®, PDL), TRX-4 (Tolerx), and NI-0401 (NovImmune). However, patients treated with each of these antibodies have experienced cytokine-release associated adverse events (moderate to severe) and sometimes viral reactivation above that typically observed in the patient population.

Given the cytokine-release associated adverse events related to current T cell antibody and other biologic therapies, there is a continuing need for alternative therapies. The present invention meets such needs, and further provides other related advantages.

### BRIEF SUMMARY

The present disclosure provide fusion proteins that bind to a TCR complex or a component thereof, compositions and unit dosage forms comprising such fusion proteins, polynucleotides and expression vectors that encode such fusion proteins, methods for reducing rejection of solid organ transplant or treating an autoimmune disease, and methods for detecting T cell activation.

In one aspect, the present disclosure provides a fusion protein, comprising, consisting essentially of, or consisting of, from amino-terminus to carboxy-terminus: (a) a binding domain that specifically binds to a TCR complex or a component thereof, (b) a linker polypeptide, (c) optionally an immunoglobulin C_{H2} region polypeptide comprising (i) an amino acid substitution at the asparagine of position 297; (ii) one or more amino acid substitutions or deletions at positions 234-238; (iii) at least one amino acid substitution or deletion at positions 253, 310, 318, 320, 322, or 331; (iv) an amino acid substitution at the asparagine of position 297 and one or more substitutions or deletions at positions 234-238; (v) an amino acid substitution at the asparagine of position 297 and at least one substitution or deletion at position 253, 310, 318, 320, 322, or 331; (vi) one or more amino acid substitutions or deletions at positions 234-238, and at least one amino acid substitution or deletion at position 253, 310, 318, 320, 322, or 331; or (vi) an amino acid substitution at the asparagine of position 297, one or more amino acid substitutions or deletions at positions 234-238, and at least one amino acid substitution or deletion at position 253, 310, 318, 320, 322, or 331, and (d) an immunoglobulin C_{H3} region polypeptide, wherein the fusion protein does not induce a cytokine storm or induces a minimally detectable cytokine release, and wherein the amino acid residues in the immunoglobulin C region are numbered by the EU numbering system. Additional fusion proteins are provided according to claims 2 to 20 and described herein.

In another aspect, the present disclosure provides a composition comprising a fusion protein provided herein and a pharmaceutically acceptable carrier, diluent, or excipient.

In another aspect, the present disclosure provides a unit dose form comprising the above-noted pharmaceutical composition.

In another aspect, the present disclosure provides a polynucleotide encoding a fusion protein provided herein.

In another aspect, the present disclosure provides an expression vector comprising a polynucleotide encoding a fusion protein provided herein that is operably linked to an expression control sequence.

In another aspect, the present disclosure provides a method of reducing rejection of solid organ transplant, comprising administering to a solid organ transplant recipient an effective amount of a fusion protein provided herein.

In another aspect, the present disclosure provides a method for treating an autoimmune disease (e.g., inflammatory bowel diseases, including Crohn's disease and ulcerative colitis, diabetes mellitus, asthma and arthritis), comprising administering to a patient in need thereof an effective amount of a fusion protein provided herein.

In another aspect, the present disclosure provides a method for detecting cytokine release induced by a protein that comprises a binding domain that specifically binds to a TCR complex or a component thereof, comprising: (a) providing mitogen-primed T cells, (b) treating the primed T cells of step (a) with the protein that comprises a binding domain that specifically binds to a TCR complex or a component thereof (e.g., a fusion protein and an antibody), and (c) detecting release of a cytokine from the primed T cells that have been treated in step (b).

In another aspect, the present disclosure provides a method for detecting T cell activation induced by a protein that comprises a binding domain that specifically binds to a TCR complex or a component thereof, comprising: (a) providing mitogen-primed T cells, (b) treating the primed T cells of step (a) with the protein that comprises a binding domain that specifically binding to a TCR complex or a component thereof (e.g., a fusion protein and an antibody), and (c) detecting activation of the primed T cells that have been treated in step (b).

### BRIEF DESCRIPTION OF THE DRAWINGS

FIG. 1 shows the percentage of activated T cells resulting from treating PHA-primed human T cells with various antibodies and small modular immunopharmaceutical (SMIP™) products. “No Rx” refers to no treatment, which was used as a negative control.

FIG. 2 shows the percentage of activated T cells resulting from treating responder cells with various antibodies and SMIP fusion proteins in a mixed lymphocyte reaction assay. “MLR” refers to mixed lymphocyte reaction without any additional treatment. “Responder only” refers to a reaction where only responder cells were present. “IgG2a” refers to responder cells treated with 10 μg/ml IgG2a mAb.

FIG. 3 shows the percentage of activated T cells resulting from treating responder cells with various antibodies and SMIP fusion proteins in a mixed lymphocyte reaction assay. “MLR” refers to mixed lymphocyte reaction without any additional treatment. “Responder only” refers to a reaction where only responder cells were present.

FIG. 4 shows the percentage of activated T cells resulting from treating memory T cells with a monoclonal antibody and various SMIP fusion proteins. “Responder (No TT)” refers to a reaction in the absence of tetanus toxoid.

FIGS. 5A and 5B are FACS analysis dot plots of TCR and CD3 on human T cells stained (A) immediately after isolation (day 0) or (b) 4 days after treatment with OKT3 monoclonal antibody or various OKT3 SMIP fusion proteins.

FIGS. 6A and 6B are FACS analysis dot plots of TCR and CD3 on human T cells stained (A) immediately after isolation (day 0) or (B) 4 days after treatment with OKT3 IgG1AA or OKT3 HM1 SMIP fusion proteins.

FIG. 7 shows changes in fluorescence of a calcium flux indicator dye over time resulting from treating purified human T cells with monoclonal antibodies, combinations of antibodies, or various OKT3 SMIP fusion proteins.

FIGS. 8A and 8B show (A) IFNγ or (B) IP-10 release after treating ConA-primed mouse T cells with monoclonal antibodies (2C11 mAb and H57 mAb) or SMIP fusion proteins (2C11 Null2 and H57 Null2).

FIG. 9 shows the percentage of activated T cells resulting from treating responder cells with various antibodies or SMIP fusion proteins in a mixed lymphocyte reaction assay. “R only” refers to a reaction having only responder cells present; “S only” refers to a reaction having only stimulator cells present; and “R:S” refers to a reaction having both responder and stimulator cells present.

FIGS. 10A and 10B show changes in (A) body weights and (B) clinical score over time post intravenous administration of antibody (H57 mAb) and H57 Null2 SMIP fusion protein at various concentrations. PBS and IgG2a were used as negative controls.

FIGS. 11A and 11B show the concentration of (A) IL-6 and (B) IL-4 in serum 2 hours, 24 hours, 72 hours after intravenous administration into normal BALB/c mice of an anti-TCR antibody (H57 mAb) or various concentrations of an anti-TCR SMIP fusion protein (H57 Null2). Mouse IgG2a antibody and PBS (diluent) were used as negative controls.

FIG. 12 shows the percentage of T cells found in a mouse spleen that were coated with H57 Null2 SMIP on days 1 or 3 after intravenous administration of various concentrations of an anti-TCR SMIP fusion protein (H57 Null2). PBS and IgG2a were used as negative controls.

FIG. 13 shows the percentage of change of initial body weight of recipient mice over 14 days following the transfer of donor cells in a model of acute Graft versus Host Disease (aGVHD). “Naïve recipient” indicates mice which received no donor cell transfer as a negative control. Recipient mice were treated with H57 Null2 SMIP fusion protein, dexamethasone (DEX), or control (PBS or IgG2a).

FIGS. 14A to 14C show the serum concentration of (A) G-CSF, (B) KC, or (C) IFNγ on day 14, day 14, or day 7, respectively, after transfer of donor cells.

FIG. 15 shows the donor:host lymphocyte ratio on day 14 after transfer of donor cells. “No cell transfer” indicates a negative control mouse that did not receive donor cells. PBS and IgG2a were used as control treatments.

FIG. 16 shows sequence alignments among the C_{H2} regions of human IgG1, human IgG2, human IgG4, and mouse IGHG2c (SEQ ID NOS:64, 66, 68 and 73, respectively). The alignments were performed using the Clustal W method with default parameters of the MegAlign program of DNASTAR 5.03 (DNASTAR Inc.). The amino acid positions of human IgG1 C_{H2} are based on the EU numbering according to Kabat (see Kabat, Sequences of Proteins of Immunological Interest, 5^{th} ed. Bethesda, Md.: Public Health Service, National Institutes of Health (1991)). That is, the heavy chain variable region of human IgG1 is deemed to be 128 amino acids in length, so the most amino-terminal amino acid residue in the constant region of human IgG1 is at position 129. The amino acid positions of other C_{H2} regions are indicated based on the positions of the amino acid residues in human IgG1 with which they align. The Asn residues at position 297 (N297) are underlined and in bold.

FIG. 17 shows the percentage of activated T cells resulting from treating responder cells with either an antibody or a SMIP fusion protein in a mixed lymphocyte reaction (MLR) assay. “R” refers to a reaction where only responder cells were present, “S” refers to a reaction where only stimulator cells were present, “R+S” refers to mixed lymphocyte reaction without any additional treatment, “muIgG2b” refers to responder cells treated with 10 μg/ml mouse IgG2b. “Control SMIP” is a SMIP fusion protein having an scFv binding domain that does not bind to T cells. The cells were tested with Cris-7 IgG1 N297A (SEQ ID NO:265).

FIG. 18 shows FACS analysis dot plots of TCR and CD3 on human T cells stained immediately after isolation. The top two panels show human T cells treated with Cris-7 monoclonal antibody and the bottom two panels show treatment with Cris-7 IgG1 N297A (SEQ ID NO:265). The panels on the left show cell distributions on the day of treatment (day 0) and the panels on the right show cell distributions 2 days after treatment (day 2).

FIG. 19 shows changes in fluorescence of a calcium flux indicator dye over time resulting from treatment of human T cells with BC3 IgG1-N297A (SEQ ID NO:80, which has Linker 87 as a hinge between the scFv and the CH2CH3 domains) compared to this same fusion protein having hinge Linker 87 swapped out for other hinges of various lengths (in particular, Linkers 115-120 and 122, which correspond to SEQ ID NOS:212-218, respectively).

FIG. 20 shows the percentage of activated T cells resulting from treating responder cells with either an antibody or a SMIP fusion protein in a MLR assay. “Control SMIP” refers to a SMIP fusion protein having an scFv binding domain that does not bind T cells. “Responder only” refers to a reaction where only responder cells were present. The numbers in brackets are the sequence identifier numbers of the SMIP fusion proteins.

FIG. 21 shows the percentage of activated T cells resulting from treating responder cells with BC3 IgG1-N297A SMIP fusion proteins containing various hinge linkers in a MLR assay.

FIG. 22 shows the percentage of activated T cells resulting from treating responder cells with monoclonal antibody Cris7, chimeric or humanized Cris7 SMIP fusion proteins, or a chimeric BC3 SMIP fusion protein (SEQ ID NO:80) in a MLR assay. “Control SMIP” refers to a SMIP fusion protein having an scFv binding domain that does not bind T cells and “Responder only” refers to a reaction where only responder cells were present. The numbers in brackets are the sequence identifier numbers of the SMIP fusion proteins.

FIG. 23 shows the percentage of activated T cells resulting from treating responder cells with humanized Cris7 IgG1-N297, IgG2-AA-N297A and IgG4-AA-N297A, and HM1 SMIP fusion proteins or chimeric Cris7 IgG1-N297A and HM1 SMIP fusion proteins in a MLR assay. “Parent mAb” refers to Cris7 mAb and “Control SMIP” refers to a SMIP fusion protein having an scFv binding domain that does not bind T cells.

FIG. 24 shows the percentage of activated T cells after PHA-primed human T cells were treated with humanized Cris7 (VH3-VL1) IgG1-N297A or humanized Cris7 (VH3-VL2) IgG1-N297A SMIP fusion proteins. “Control SMIP” is a non-T cell binding SMIP fusion protein.

FIGS. 25A and 25B show the concentration of (A) IFNγ and (B) IL-17 in serum 24 hours (day 1) and 72 hours (day 3) after restimulation of PHA-primed T cells with various humanized and chimeric Cris7 SMIP fusion proteins, BC3 SMIP fusion protein (SEQ ID NO:80), and various antibodies (BC3 mAb, parent Cris7 mAb, and Nuvion FL). The numbers in brackets are the sequence identifier numbers of the SMIP fusion proteins.

FIGS. 26A to 26H show the level of (A) IFNγ, (B) IL-10, (C) IL-1B, (D) IL-17, (E) IL-4, (F) TNF-α, (G) IL-6, and (H) IL-2 in primary PBMC treated for 24 hours (d1), 48 hours (d2), or 72 hours (d3) with humanized Cris7 (VH3-VL1) IgG4-AA-N297A SMIP fusion protein, humanized Cris7 (VH3-VL2) IgG4-AA-N297A SMIP fusion protein, or Cris7 mAb.

FIG. 27 shows changes in body weights over time post intravenous administration of IgG2a mAb (411 μg), H57 mAb (5 μg), H57 Null2 SMIP fusion protein (300 μg), H57 half null SMIP fusion protein (300 μg), or H57 HM2 SMIP fusion protein (300 μg).

FIG. 28 shows peripheral blood T cell concentrations 2 hours post intravenous administration of IgG2a mAb, H57 mAb, H57 Null2, H57 half null, or H57 HM2 as dosed in FIG. 27.

FIG. 29 shows peripheral T cell concentrations 72 hours post intravenous administration of IgG2a mAb, H57 mAb, H57 Null2, H57 half null, or H57 HM2 as dosed in FIG. 27.

FIGS. 30A to 30C show the concentration of IL-2 in serum (A) 2 hours, (B) 24 hours, and (C) 72 hours after intravenous administration of IgG2a mAb, H57 mAb, H57 Null2, H57 half null, or H57 HM2 as dosed in FIG. 27.

FIGS. 31A to 31C show the concentration of IL-10 in serum (A) 2 hours, (B) 24 hours, and (C) 72 hours after intravenous administration of IgG2a mAb, H57 mAb, H57 Null2, H57 half null, or H57 HM2 as dosed in FIG. 27.

FIGS. 32A to 32C show the concentration of IP-10 in serum (A) 2 hours, (B) 24 hours, and (C) 72 hours after intravenous administration of IgG2a mAb, H57 mAb, H57 Null2, H57 half null, or H57 HM2 as dosed in FIG. 27.

FIGS. 33A to 33C show the concentration of TNFα in serum (A) 2 hours, (B) 24 hours, and (C) 72 hours after intravenous administration of IgG2a mAb, H57 mAb, H57 Null2, H57 half null, or H57 HM2 as dosed in FIG. 27.

FIGS. 34A to 34C show the concentration of IL-4 in serum (A) 2 hours, (B) 24 hours, and (C) 72 hours after intravenous administration of IgG2a mAb, H57 mAb, H57 Null2, H57 half null, or H57 HM2 as dosed in FIG. 27.

FIGS. 35A to 35C show the concentration of MCP-1 in serum (A) 2 hours, (B) 24 hours, and (C) 72 hours after intravenous administration of IgG2a mAb, H57 mAb, H57 Null2, H57 half null, or H57 HM2 as dosed in FIG. 27.

FIGS. 36A to 36C show the concentration of KC in serum (A) 2 hours, (B) 24 hours, and (C) 72 hours after intravenous administration of IgG2a mAb, H57 mAb, H57 Null2, H57 half null, or H57 HM2 as dosed in FIG. 27.

FIGS. 37A to 37C show the concentrations of IL-17 2 hours (A), 24 hours (B) and 72 hours (C) after intravenous administration of IgG2a, H57 mAb and H57 Null2, half null and HM2 SMIPs.

FIGS. 38A to 38C show the concentration of IL-5 in serum (A) 2 hours, (B) 24 hours, and (C) 72 hours after intravenous administration of IgG2a mAb, H57 mAb, H57 Null2, H57 half null, or H57 HM2 as dosed in FIG. 27.

FIGS. 39A and 39B are graphs of the mean serum concentration versus time for H57-HM2 and H57 half null. The results are expressed as the observed data set and the predicted values calculated by WinNonLin™ software. The Rsq value and Rsq adjusted values are the goodness of fit statistics for the terminal elimination phase, before and after adjusting for the number of points used in the estimation of HL Lambda z (6.6 and 40.7 hours).

FIG. 40 shows the concentration of G-CSF in serum 15 minutes, 2 hours, 6 hours, 24 hours and 48 hours post intravenous administration of H57-HM2 or H57 Null2 (200 μg each).

FIG. 41 shows the concentration of IFN-γ in serum 15 minutes, 2 hours, 6 hours, 24 hours and 48 hours post intravenous administration of H57-HM2 or H57 Null2 (200 μg each).

FIG. 42 shows the concentration of IL-2 in serum 15 minutes, 2 hours, 6 hours, 24 hours and 48 hours post intravenous administration of H57-HM2 or H57 Null2 (200 μg each).

FIG. 43 shows the concentration of IL-5 in serum 15 minutes, 2 hours, 6 hours, 24 hours and 48 hours post intravenous administration of H57-HM2 or H57 Null2 (200 μg each).

FIG. 44 shows the concentration of IL-6 in serum 15 minutes, 2 hours, 6 hours, 24 hours and 48 hours post intravenous administration of H57-HM2 or H57 Null2 (200 μg each).

FIG. 45 shows the concentration of IL-10 in serum 15 minutes, 2 hours, 6 hours, 24 hours and 48 hours post intravenous administration of H57-HM2 or H57 Null2 (200 μg each).

FIG. 46 shows the concentration of IL-17 in serum 15 minutes, 2 hours, 6 hours, 24 hours and 48 hours post intravenous administration of H57-HM2 or H57 Null2 (200 μg each).

FIG. 47 shows the concentration of IP-10 in serum 15 minutes, 2 hours, 6 hours, 24 hours and 48 hours post intravenous administration of H57-HM2 or H57 Null2 (200 μg each).

FIG. 48 shows the concentration of KC 15 in serum minutes, 2 hours, 6 hours, 24 hours and 48 hours post intravenous administration of H57-HM2 or H57 Null2 (200 μg each).

FIG. 49 shows the concentration of MCP-1 in serum 15 minutes, 2 hours, 6 hours, 24 hours and 48 hours post intravenous administration of H57-HM2 or H57 Null2 (200 μg each).

FIG. 50 shows the percentage of activated T cells resulting from treating responder cells with H57 Null2, H57 half null, H57-HM2, mouse IgG2a mAb, or H57 mAb.

FIG. 51 shows the percentage of activated T cells resulting from treating responder cells with H57 Null2, H57 half null, H57-HM2, or H57 mAb normalized to (R+S)−without treatment=100%.

FIG. 52 shows the percentage of ConA-primed T cells activated by treatments of H57 Null2, H57 half null, H57-HM2, mouse IgG2a mAb, H57 mAb, or 2C11 mAb.

### DETAILED DESCRIPTION

The present disclosure provides fusion proteins containing one or more binding domains directed against the TCR complex in the form of small modular immunopharmaceutical (SMIP™) products or in the form of a SMIP molecule SMIP molecule with Fc and binding domain in the reverse N-terminal to C-terminal orientation (PIMS) that induce a unique T cell signaling profile. This signaling profile is characterized by an undetectable or small, minimal, or nominal cytokine release (i.e., absence of or minimal cytokine storm), induction of calcium flux, phosphorylation of TCR signaling proteins without activating T cells, or any combination thereof. Such a signaling profile is not replicated using monoclonal antibodies, demonstrating an unexpected signaling signature caused by the binding of SMIP or PIMS proteins to their targets. To date, protein molecules directed against the TCR complex either induce a strong T cell signal (e.g., cytokine storm) together with T cell activation or have little effect on cells in the absence of cross-linking.

Furthermore, this disclosure provides nucleic acid molecules that encode such fusion proteins, as well as vectors and host cells for recombinantly producing such proteins, and compositions and methods for using the fusion proteins of this disclosure in various therapeutic applications, including the treatment as well as the amelioration of at least one symptom of a disease or condition (e.g., an autoimmune disease, inflammatory disease, and organ transplant rejection).

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, “about” or “consisting essentially of” means±20% of the indicated range, value, or structure, unless otherwise indicated. As used herein, the terms “include” and “comprise” are used synonymously. It should be understood that the terms “a” and “an” as used herein refer to “one or more” of the enumerated components. The use of the alternative (e.g., “or”) should be understood to mean either one, both, or any combination thereof of the alternatives. In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present invention.

The amino acid residues in immunoglobulin C_{H2} and C_{H3} regions of the present disclosure are numbered by the EU numbering system unless otherwise indicated (see, Kabat et al., Sequences of Proteins of Immunological Interest, 5^{th} ed. Bethesda, Md.: Public Health Service, National Institutes of Health (1991)).

A “small modular immunopharmaceutical (SMIP™) protein” refers to a single chain fusion protein that comprises from its amino to carboxy terminus: a binding domain that specifically binds a target molecule, a linker polypeptide (e.g., an immunoglobulin hinge or derivative thereof), an immunoglobulin C_{H2} polypeptide and an immunoglobulin C_{H3} polypeptide (see, U.S. Patent Publication Nos. 2003/0133939, 2003/0118592, and 2005/0136049).

A “PIMS protein” is a reverse SMIP molecule wherein the binding domain is disposed at the carboxy-terminus of the fusion protein. Constructs and methods for making PIMS proteins are described in PCT Publication No. WO 2009/023386. In general, a PIMS molecule is a single-chain polypeptide comprising, in amino-terminal to carboxy-terminal orientation, an optional C_{H2} region polypeptide a C_{H3} domain, a linker peptide (e.g., an immunoglobulin hinge region), and a specific binding domain.

As used herein, a protein “consists essentially of” several domains (e.g., a binding domain that specifically binds a TCR complex or a component thereof, a linker polypeptide, an immunoglobulin C_{H2} region, and an immunoglobulin C_{H3} region) if the other portions of the protein (e.g., amino acids at the amino- or carboxy-terminus or between two domains), in combination, contribute to at most 20% (e.g., at most 15%, 10%, 8%, 6%, 5%, 4%, 3%, 2% or 1%) of the length of the protein and do not substantially affect (i.e., do not reduce the activity by more than 50%, such as more than 40%, 30%, 25%, 20%, 15%, 10%, or 5%) the activities of the protein, such as the affinity to a TCR complex or a component thereof, the ability to not induce (or induce a minimally detectable) cytokine release, the ability to induce calcium flux or phosphorylation of a molecule in the T cell receptor signaling pathway, the ability to block T cell response to an alloantigen, the ability to block memory T cell response to an antigen, and down-modulating the TCR complex of the cell. In certain embodiments, a fusion protein consists essentially of a binding domain that specifically binds a TCR complex or a component thereof, a linker polypeptide, an optional immunoglobulin C_{H2} region polypeptide, and an immunoglobulin C_{H3} region polypeptide. Such molecules may further comprise junction amino acids at the amino- or carboxy-terminus of the protein or between two different domains (e.g., between the binding domain and the linker polypeptide, between the linker polypeptide and the immunoglobulin C_{H2} region polypeptide, or between the immunoglobulin C_{H2} region polypeptide and the immunoglobulin C_{H3} region polypeptide).

Terms understood by those in the art of antibody technology are each given the meaning acquired in the art, unless expressly defined differently herein. Antibodies are known to have variable regions, a hinge region, and constant domains. Immunoglobulin structure and function are reviewed, for example, in Harlow et al., Eds., Antibodies: A Laboratory Manual, Chapter 14 (Cold Spring Harbor Laboratory, Cold Spring Harbor, 1988). For example, the terms “VL” and “VH” refer to the variable binding region from an antibody light and heavy chain, respectively. The variable binding regions are made up of discrete, well-defined sub-regions known as “complementarity determining regions” (CDRs) and “framework regions” (FRs). The term “CL” refers to an “immunoglobulin light chain constant region” or a “light chain constant region,” i.e., a constant region from an antibody light heavy chain. The term “CH” refers to an “immunoglobulin heavy chain constant region” or a “heavy chain constant region,” which is further divisible, depending on the antibody isotype into C_{H1}, C_{H2}, and C_{H3} (IgA, IgD, IgG), or C_{H1}, C_{H2}, C_{H3}, and C_{H4} domains (IgE, IgM). A portion of the constant region domains make up the Fc region (the “fragment crystallizable” region) from an antibody and is responsible for the effector functions of an immunoglobulin, such as ADCC (antibody-dependent cell-mediated cytotoxicity), ADCP (antibody-dependent cellular phagocytosis), CDC (complement-dependent cytotoxicity) and complement fixation, binding to Fc receptors (e.g., CD16, CD32, FcRn), greater half-life in vivo relative to a polypeptide lacking an Fc region, protein A binding, and perhaps even placental transfer (see Capon et al., Nature, 337:525 (1989)).

In addition, antibodies have a hinge sequence that is typically situated between the Fab and Fc region (but a lower section of the hinge may include an amino-terminal portion of the Fc region). By way of background, an immunoglobulin hinge acts as a flexible spacer to allow the Fab portion to move freely in space. In contrast to the constant regions, hinges are structurally diverse, varying in both sequence and length between immunoglobulin classes and even among subclasses. For example, a human IgG1 hinge region is freely flexible, which allows the Fab fragments to rotate about their axes of symmetry and move within a sphere centered at the first of two inter-heavy chain disulfide bridges. By comparison, a human IgG2 hinge is relatively short and contains a rigid poly-proline double helix stabilized by four inter-heavy chain disulfide bridges, which restricts the flexibility. A human IgG3 hinge differs from the other subclasses by its unique extended hinge region (about four times as long as the IgG1 hinge), containing 62 amino acids (including 21 prolines and 11 cysteines), forming an inflexible poly-proline double helix and providing greater flexibility because the Fab fragments are relatively far away from the Fc fragment. A human IgG4 hinge is shorter than IgG1 but has the same length as IgG2, and its flexibility is intermediate between that of IgG1 and IgG2.

According to crystallographic studies, an IgG hinge domain can be functionally and structurally subdivided into three regions: the upper, the core or middle, and the lower hinge regions (Shin et al., Immunological Reviews 130:87 (1992)). Exemplary upper hinge regions include EPKSCDKTHT (SEQ ID NO:359) as found in IgG1, ERKCCVE (SEQ ID NO:360) as found in IgG2, ELKTPLGDTT HT (SEQ ID NO:361) or EPKSCDTPPP (SEQ ID NO:362) as found in IgG3, and ESKYGPP (SEQ ID NO:363) as found in IgG4. Exemplary middle or core hinge regions include CPPCP (SEQ ID NO:364) as found in IgG1 and IgG2, CPRCP (SEQ ID NO:365) as found in IgG3, and CPSCP (SEQ ID NO:366) as found in IgG4. While IgG1, IgG2, and IgG4 antibodies each appear to have a single upper and middle hinge, IgG3 has four in tandem—one being ELKTPLGDTTHTCPRCP (SEQ ID NO:367) and three being EPKSCDTPPPCPRCP (SEQ ID NO:368).

IgA and IgD antibodies appear to lack an IgG-like core region, and IgD appears to have two upper hinge regions in tandem (see, ESPKAQASSVPTAQPQAEGSLAKATTAPATTRNT (SEQ ID NO:369) and GRGGEEKKKEKEKEEQEERETKTP (SEQ ID NO:370). Exemplary wild type upper hinge regions found in IgA1 and IgA2 antibodies are VPSTPPTPSPSTPPTPSPS (SEQ ID NO:371) and VPPPPP (SEQ ID NO:372), respectively.

IgE and IgM antibodies, in contrast, lack a typical hinge region and instead have a C_{H2} domain with hinge-like properties. Exemplary wild-type C_{H2} upper hinge-like sequences of IgE and IgM are set forth in SEQ ID NO:373 (VCSRDFTPPTVKILQSSSDGGGHFPPTIQLLCLVSGYTPGTINITWLEDG QVMDVDLSTASTTQEGELASTQSELTLSQKHWLSDRTYTCQVTYQGHTFE DSTKKCA) and SEQ ID NO:374 (VIAELPPKVSVFVPPRDGFFGNPRKSKLIC QATGFSPRQIQVSWLREGKQVGSGVTTDQVQAEAKESGPTTYKVTSTLTI KESDWLGQSMFTCRVDHRGLTFQQNASSMCVP), respectively.

As used herein, a “hinge region” or a “hinge” refers to (a) an immunoglobulin hinge region (made up of, for example, upper and core regions) or a functional variant thereof, (b) a lectin interdomain region or a functional variant thereof, or (c) a cluster of differentiation (CD) molecule stalk region or a functional variant thereof.

An immunoglobulin hinge region may be a wild type immunoglobulin hinge region or an altered wild type immunoglobulin hinge region or altered immunoglobulin hinge region.

As used herein, a “wild type immunoglobulin hinge region” refers to a naturally occurring upper and middle hinge amino acid sequences interposed between and connecting the C_{H1} and C_{H2} domains (for IgG, IgA, and IgD) or interposed between and connecting the C_{H1} and C_{H3} domains (for IgE and IgM) found in the heavy chain of an antibody.

An “altered wild type immunoglobulin hinge region” or “altered immunoglobulin hinge region” refers to (a) a wild type immunoglobulin hinge region with up to 30% amino acid changes (e.g., up to 25%, 20%, 15%, 10%, or 5% amino acid substitutions or deletions), or (b) a portion of a wild type immunoglobulin hinge region that has a length of about 5 amino acids (e.g., about 5, 6, 7, 8, 9, 10, 11, 12, 13, 14, 15, 16, 17, 18, 19, or 20 amino acids) up to about 120 amino acids (preferably having a length of about 10 to about 40 amino acids or about 15 to about 30 amino acids or about 15 to about 20 amino acids or about 20 to about 25 amino acids), has up to about 30% amino acid changes (e.g., up to about 25%, 20%, 15%, 10%, 5%, 4%, 3%, 2%, or 1% amino acid substitutions or deletions or a combination thereof), and has an IgG core hinge region as set forth in SEQ ID NOS:364, 365, or 366.

A “variable domain linking sequence” is an amino acid sequence that connects a heavy chain variable region to a light chain variable region and provides a spacer function compatible with interaction of the two sub-binding domains so that the resulting polypeptide retains a specific binding affinity to the same target molecule as an antibody that comprises the same light and heavy chain variable regions. In certain embodiments, a hinge useful for linking a binding domain to an immunoglobulin C_{H2} or C_{H3} region polypeptide may be used as a variable domain linking sequence.

A “linker polypeptide” refers to an amino acid sequence that links a binding domain to an immunoglobulin C_{H2} or C_{H3} region polypeptide in a fusion protein. In certain embodiments, the linker polypeptide is a hinge as defined herein. In certain embodiments, a variable domain linking sequence useful for connecting a heavy chain variable region to a light chain variable region may be used as a linker polypeptide.

In certain embodiments, there may be one or a few (e.g., 2-8) amino acid residues between two domains of a fusion protein, such as between a binding domain and a linker polypeptide, between a linker polypeptide and an immunoglobulin C_{H2} region polypeptide, and between an immunoglobulin C_{H2} region polypeptide and an immunoglobulin C_{H3} region polypeptide, such as amino acid residues resulting from construct design of the fusion protein (e.g., amino acid residues resulting from the use of a restriction enzyme site during the construction of a nucleic acid molecule encoding a single chain polypeptide). As described herein, such amino acid residues may be referred to “junction amino acids” or “junction amino acid residues.”

“Derivative” as used herein refers to a chemically or biologically modified version of a compound (e.g., a protein) that is structurally similar to a parent compound and (actually or theoretically) derivable from that parent compound.

As used herein, “amino acid” refers to a natural amino acid (those occurring in nature), a substituted natural amino acid, a non-natural amino acid, a substituted non-natural amino acid, or any combination thereof. The designations for natural amino acids are herein set forth as either the standard one- or three-letter code. Natural polar amino acids include asparagine (Asp or N) and glutamine (Gln or Q); as well as basic amino acids such as arginine (Arg or R), lysine (Lys or K), histidine (His or H), and derivatives thereof and acidic amino acids such as aspartic acid (Asp or D) and glutamic acid (Glu or E), and derivatives thereof. Natural hydrophobic amino acids include tryptophan (Trp or W), phenylalanine (Phe or F), isoleucine (Ile or I), leucine (Leu or L), methionine (Met or M), valine (Val or V), and derivatives thereof as well as other non-polar amino acids such as glycine (Gly or G), alanine (Ala or A), proline (Pro or P), and derivatives thereof. Natural amino acids of intermediate polarity include serine (Ser or S), threonine (Thr or T), tyrosine (Tyr or Y), cysteine (Cys or C), and derivatives thereof. Unless specified otherwise, any amino acid described herein may be in either the D- or L-configuration.

Amino acids can be classified according to physical properties and contribution to secondary and tertiary protein structure. A “conservative substitution” is recognized in the art as a substitution of one amino acid for another amino acid that has similar properties. Exemplary conservative substitutions are well known in the art (see, e.g., WO 97/09433, page 10, published Mar. 13, 1997; Lehninger, Biochemistry, Second Edition; Worth Publishers, Inc. NY:NY (1975), pp. 71-77; Lewin, Genes IV, Oxford University Press, NY and Cell Press, Cambridge, Mass. (1990), p. 8. In certain embodiments, a conservative substitution includes a leucine to serine substitution.

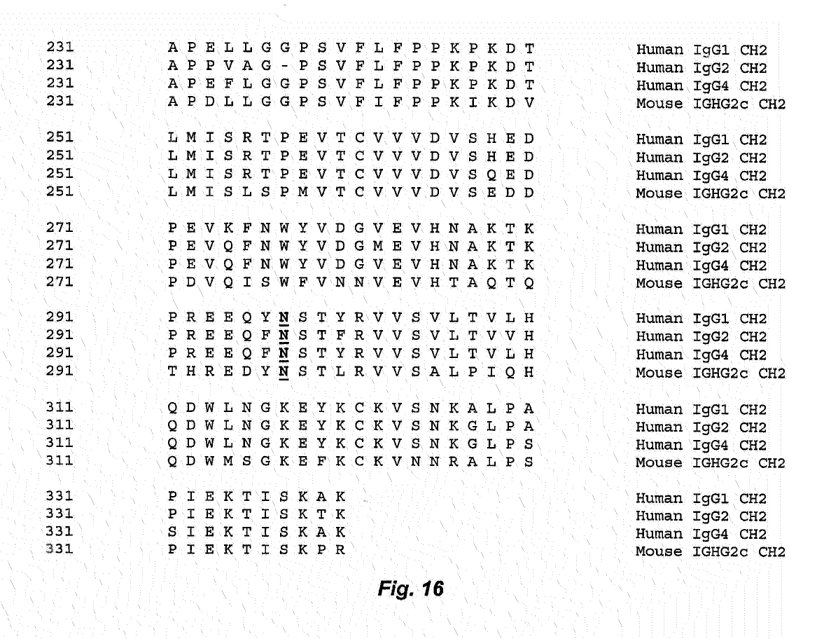

As used herein, unless otherwise provided, a position of an amino acid residue in the constant region of human IgG1 heavy chain is numbered assuming that the variable region of human IgG1 is composed of 128 amino acid residues according to the Kabat numbering convention. The numbered constant region of human IgG1 heavy chain is then used as a reference for numbering amino acid residues in constant regions of other immunoglobulin heavy chains. A position of an amino acid residue of interest in a constant region of an immunoglobulin heavy chain other than human IgG1 heavy chain is the position of the amino acid residue in human IgG1 heavy chain with which the amino acid residue of interest aligns. Alignments between constant regions of human IgG1 heavy chain and other immunoglobulin heavy chains may be performed using software programs known in the art, such as the Megalign program (DNASTAR Inc.) using the Clustal W method with default parameters. Exemplary sequence alignments are shown in FIG. 16. According to the numbering system described herein, although human IgG2 C_{H2} region has an amino acid deletion near its amino-terminus compared with other C_{H2} regions in FIG. 16, the position of the underlined “N” in human IgG2 C_{H2} is still position 297, because this residue aligns with “N” at position 297 in human IgG1 C_{H2}.

### Fusion Proteins Directed Against TCR Complex

In one aspect, the present disclosure provides a single chain fusion protein in the form of a SMIP fusion protein that comprises, consists essentially of, or consists of, from its amino-terminus to its carboxy-terminus: (a) a binding domain that specifically binds to a TCR complex or a component thereof, (b) a linker polypeptide, (c) optionally an immunoglobulin C_{H2} region polypeptide, and (d) an immunoglobulin C_{H3} region polypeptide. The immunoglobulin C_{H2} region polypeptide when present may comprise (1) an amino acid substitution at the asparagine of position 297; (2) one or more amino acid substitutions or deletions at positions 234-238; (3) at least one amino acid substitution or deletion at positions 253, 310, 318, 320, 322, or 331; (4) an amino acid substitution at the asparagine of position 297 and one or more substitutions or deletions at positions 234-238; (5) an amino acid substitution at the asparagine of position 297 and one or more substitutions or deletions at positions 253, 310, 318, 320, 322, or 331; (6) one or more amino acid substitutions or deletions at positions 234-238, 253, 310, 318, 320, 322, or 331; or (7) an amino acid substitution at the asparagine of position 297 and at least one amino acid substitution or deletion at positions 234-238, 253, 310, 318, 320, 322, or 331.

In preferred embodiments, a single chain fusion protein of this disclosure will comprise, consist essentially of, or consist of, from its amino-terminus to its carboxy-terminus: (a) a binding domain that specifically binds to a TCR complex or a component thereof, (b) a linker polypeptide, (c) an immunoglobulin C_{H2} region polypeptide, and (d) an immunoglobulin C_{H3} region polypeptide, wherein the immunoglobulin C_{H2} region polypeptide comprises (i) an amino acid substitution at the asparagine of position 297 and one or more substitutions or deletions at positions 234-238; (ii) an amino acid substitution at the asparagine of position 297, a substitution at positions 234, 235, and 237, and a deletion at position 236; (iii) at least one amino acid substitution or deletion at positions 234-238, 253, 310, 318, 320, 322, or 331; (iv) an amino acid substitution at positions 234, 235, 237, 318, 320, and 322, and a deletion at position 236; (v) an amino acid substitution at the asparagine of position 297 and at least one substitution or deletion at positions 234-238, 253, 310, 318, 320, 322, or 331; or (vi) an amino acid substitution at the asparagine of position 297, an amino acid substitution at positions 234, 235, 237, 318, 320, and 322, and a deletion at position 236. In each of these preferred embodiments, the amino acid used in the substation is preferably alanine or serine.

In further preferred embodiments, a single chain fusion protein of this disclosure will comprise, consist essentially of, or consist of, from its amino-terminus to its carboxy-terminus: (a) a binding domain that specifically binds to a TCR complex or a component thereof, (b) a linker polypeptide, and (c) an immunoglobulin C_{H3} region polypeptide, wherein the immunoglobulin C_{H3} region polypeptide comprises a C_{H3} region of human IgM and a C_{H3} region of human IgG (preferably IgG1).

The fusion proteins will only undetectably, nominally, minimally, or at a low level induce cytokine release (i.e., cytokine storm), or will activate T cells, and may additionally be capable of one or more of the following activities: (1) inducing calcium flux, (2) inducing phosphorylation of molecules in the TCR signaling pathway, (3) blocking T cell response to an alloantigen, (4) blocking memory T cell response to an antigen, and (5) downmodulating the TCR complex.

In a preferred embodiment, the fusion protein comprises an amino acid sequence as set forth in SEQ ID NO:293, 294, 298, or 299. In related preferred embodiments, the hinge sequence at amino acids 247 to 261 of SEQ ID NOS:293, 294, 298, and 299 is replaced with a hinge amino acid sequence as set forth in SEQ ID NOS:379-434. In further preferred embodiments, the immunoglobulin C_{H2} region polypeptide of SEQ ID NOS:293, 294, 298, and 299 further comprises amino acid substitutions at positions 318, 320, and 322 according to EU numbering.

In a related aspect, the present disclosure provides a single chain fusion protein in the form of a PIMS protein that comprises, consists essentially of, or consists of, from its amino-terminus to its carboxy-terminus: (a) optionally an immunoglobulin C_{H2} region polypeptide, (b) an immunoglobulin C_{H3} region polypeptide, (c) a linker polypeptide, and (d) a binding domain that specifically binds to a TCR complex or a component thereof. The immunoglobulin C_{H2} region polypeptide when present may comprise the same types of mutations as in the SMIP fusion proteins provided herein. In addition, the PIMS proteins will have one or more of the desirable biological activities that a SMIP fusion protein, as described herein, has.

### Binding Domain

As described herein, a fusion protein of the present disclosure comprises a binding domain that specifically binds to a TCR complex or a component thereof (such as CD3, TCRα, TCRβ, or any combination thereof).

A “binding domain” or “binding region” according to the present disclosure may be, for example, any protein, polypeptide, oligopeptide, or peptide that possesses the ability to specifically recognize and bind to a biological molecule (e.g., a TCR complex or a component thereof). A binding domain includes any naturally occurring, synthetic, semi-synthetic, or recombinantly produced binding partner for a biological molecule of interest. For example, a binding domain may be antibody light chain and heavy chain variable domain regions, or the light and heavy chain variable domain regions can be joined together in a single chain and in either orientation (e.g., VL-VH or VH-VL). A variety of assays are known for identifying binding domains of the present disclosure that specifically bind with a particular target, including Western blot, ELISA, flow cytometry, or Biacore™ analysis.

A binding domain (or a fusion protein thereof) “specifically binds” to a target molecule if it binds to or associates with a target molecule with an affinity or Ka (i.e., an equilibrium association constant of a particular binding interaction with units of 1/M) of, for example, greater than or equal to about 10⁵ M⁻¹. In certain embodiments, a binding domain (or a fusion protein thereof) binds to a target with a Ka greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding domains (or single chain fusion proteins thereof) refers to those binding domains with a Kₐ of at least 10⁷ M⁻¹, at least 10⁸ M⁻¹, at least 10⁹ M⁻¹, at least 10¹⁰ M⁻¹ at least 10¹¹ M⁻¹ at least 10¹² M⁻¹ at least 10¹³ M⁻¹, or greater. Alternatively, affinity may be defined as an equilibrium dissociation constant (K_{d}) of a particular binding interaction with units of M (e.g., 10⁻⁵ M to 10⁻¹³ M, or less). Affinities of binding domain polypeptides and fusion proteins according to the present disclosure can be readily determined using conventional techniques (see, e.g., Scatchard et al. (1949) Ann. N.Y. Acad. Sci. 51:660; and U.S. Pat. Nos. 5,283,173; 5,468,614, or the equivalent).

“T cell receptor” (TCR) is a molecule found on the surface of T cells that, along with CD3, is generally responsible for recognizing antigens bound to major histocompatibility complex (MHC) molecules. It consists of a disulfide-linked heterodimer of the highly variable α and β chains in most T cells. In other T cells, an alternative receptor made up of variable γ and δ chains is expressed. Each chain of the TCR is a member of the immunoglobulin superfamily and possesses one N-terminal immunoglobulin variable domain, one immunoglobulin constant domain, a transmembrane region, and a short cytoplasmic tail at the C-terminal end (see, Abbas and Lichtman, Cellular and Molecular Immunology (5th Ed.), Editor: Saunders, Philadelphia, 2003; Janeway et al., Immunobiology: The Immune System in Health and Disease, 4^{th} Ed., Current Biology Publications, p148, 149, and 172, 1999). TCR as used in the present disclosure may be from various animal species, including human, mouse, rat, or other mammals.

“Anti-TCR fusion protein, SMIP, or antibody” refers to a fusion protein, SMIP, or antibody that specifically binds to a TCR molecule or one of its individual chains (e.g., TCR α, TCRβ, TCRγ or TCRδ chain). In certain embodiments, an anti-TCR fusion protein, SMIP, or antibody specifically binds to a TCR α, a TCRβ, or both.

“CD3” is known in the art as a multi-protein complex of six chains (see, Abbas and Lichtman, 2003; Janeway et al., p172 and 178, 1999). In mammals, the complex comprises a CD3γ chain, a CD3δchain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. Without wishing to be bound by theory, it is believed the ITAMs are important for the signaling capacity of a TCR compelx. CD3 as used in the present disclosure may be from various animal species, including human, mouse, rat, or other mammals.

“Anti-CD3 fusion protein, SMIP, or antibody,” as used herein, refers to a fusion protein, SMIP, or antibody that specifically binds to individual CD3 chains (e.g., CD3γ chain, CD3δ chain, CD3ε chain) or a complex formed from two or more individual CD3 chains (e.g., a complex of more than one CD3ε chains, a complex of a CD3γ and CD3ε chain, a complex of a CD3δ and CD3ε chain). In certain preferred embodiments, an anti-CD3 fusion protein, SMIP, or antibody specifically binds to a CD3γ, a CD3δ, a CD3ε, or any combination thereof, and more preferably a CD3ε.

“TCR complex,” as used herein, refers to a complex formed by the association of CD3 with TCR. For example, a TCR complex can be composed of a CD3γ chain, a CD3δ chain, two CD3ε chains, a homodimer of CD3ε chains, a TCRα chain, and a TCRβ chain. Alternatively, a TCR complex can be composed of a CD3γ chain, a CD3δ chain, two CD3ε chains, a homodimer of CD3ζ chains, a TCRγ chain, and a TCRδ chain.

“A component of a TCR complex,” as used herein, refers to a TCR chain (i.e., TCRα, TCRβ, TCRγ or TCRδ), a CD3 chain (i.e., CD3γ, CD3δ, CD3ε or CD3C), or a complex formed by two or more TCR chains or CD3 chains (e.g., a complex of TCRα and TCRβ, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains).

By way of background, the TCR complex is generally responsible for initiating a T cell response to antigen bound to MHC molecules. It is believed that binding of a peptide:MHC ligand to the TCR and a co-receptor (i.e., CD4 or CD8) brings together the TCR complex, the co-receptor, and CD45 tyrosine phosphatase. This allows CD45 to remove inhibitory phosphate groups and thereby activate Lck and Fyn protein kinases. Activation of these protein kinases leads to phosphorylation of the ITAM on the CD3C chains, which in turn renders these chains capable of binding the cytosolic tyrosine kinase ZAP-70. The subsequent activation of bound ZAP-70 by phosphorylation triggers three signaling pathways, two of which are initiated by the phosphorylation and activation of PLC-γ, which then cleaves phoshatidylinositol phosphates (PIPs) into diacylglycerol (DAG) and inositol trisphosphate (IP₃). Activation of protein kinase C by DAG leads to activation of the transcription factor NFκB. The sudden increase in intracellular free Ca²⁺ as a result of IP₃ action activates a cytoplasmic phosphatase, calcineurin, which enables the transcription factor NFAT (nuclear factor of activated T cells) to translocate form the cytoplasm to the nucleus. Full transcriptional activity of NFAT also requires a member of the AP-1 family of transcription factors; dimers of members of the Fos and Jun families of transcription regulators. A third signaling pathway initiated by activated ZAP-70 is the activation of Ras and subsequent activation of a MAP kinase cascade. This culminates in the activation of Fos and hence of the AP-1 transcription factors. Together, NFκB, NFAT, and AP-1 act on the T cell chromosomes, initiating new gene transcription that results in the differentiation, proliferation and effector actions of T cells. See, Janeway et al., p178, 1999.

In certain embodiments, a binding domain of the present disclosure specifically binds to an individual CD3 chain (e.g., CD3γ, CD3δ, or CD3ε) or a combination of two or more of the individual CD3 chains (e.g., a complex formed from CD3γ and CD3ε or a complex formed from CD3δ and CD3ε). In certain embodiments, the binding domain specifically binds to an individual human CD3 chain (e.g., human CD3γ chain, human CD3δ chain, and human CD3ε chain) or a combination of two or more of the individual human CD3 chains (e.g., a complex of human CD3γ and human CD3ε or a complex of human CD3δ and human CD3ε). In certain preferred embodiments, the binding domain specifically binds to a human CD3ε chain.

In certain other embodiments, a binding domain of the present disclosure specifically binds to TCRα, TCRβ, or a heterodimer formed from TCRα and TCRβ. In certain preferred embodiments, a binding domain specifically binds to one or more of human TCRα, human TCRβ, or a heterodimer formed from human TCRα and human TCRβ.

In certain embodiments, a binding domain of the present disclosure binds to a complex formed from one or more CD3 chains with one or more TCR chains, such as a complex formed from a CD3γ chain, a CD3δ chain, a CD3ε chain, a TCRα chain, or a TCRβ chain, or any combination thereof. In other embodiments, a binding domain of the present disclosure binds to a complex formed from one CD3γ chain, one CD3δ chain, two CD3ε chains, one TCRα chain, and one TCRβ chain. In further preferred embodiments, a binding domain of the present disclosure binds to a complex formed from one or more human CD3 chains with one or more human TCR chains, such as a complex formed from a human CD3γ chain, a human CD3δ chain, a human CD3ε, a human TCRα chain, or a human TCRβ chain, or any combination thereof. In certain embodiments, a binding domain of the present disclosure binds to a complex formed from one human CD3γ chain, one human CD3δ chain, two human CD3ε chains, one human TCRα chain, and one human TCRβ chain.

Binding domains of this disclosure can be generated as described herein or by a variety of methods known in the art (see, e.g., U.S. Pat. Nos. 6,291,161; 6,291,158). Sources of binding domains include antibody variable domain nucleic acid sequences from various species (which can be formatted as antibodies, sFvs, scFvs or Fabs, such as in a phage library), including human, camelid (from camels, dromedaries, or llamas; Hamers-Casterman et al. (1993) Nature, 363:446 and Nguyen et al. (1998) J. Mol. Biol., 275:413), shark (Roux et al. (1998) Proc. Nat'l. Acad. Sci. (USA) 95:11804), fish (Nguyen et al. (2002) Immunogenetics, 54:39), rodent, avian, or ovine. Exemplary anti-CD3 antibodies from which the binding domain of this disclosure may be derived include Cris-7 monoclonal antibody (Reinherz, E. L. et al. (eds.), Leukocyte typing II., Springer Verlag, New York, (1986)), BC3 monoclonal antibody (Anasetti et al. (1990) J. Exp. Med. 172:1691), OKT3 (Ortho multicenter Transplant Study Group (1985) N. Engl. J. Med. 313:337) and derivatives thereof such as OKT3 ala-ala (Herold et al. (2003) J. Clin. Invest. 11:409), visilizumab (Carpenter et al. (2002) Blood 99:2712), and 145-2C11 monoclonal antibody (Hirsch et al. (1988) J. Immunol. 140: 3766). An exemplary anti-TCR antibody is H57 monoclonal antibody (Lavasani et al. (2007) Scandinavian Journal of Immunology 65:39-47).

An alternative source of binding domains of this disclosure includes sequences that encode random peptide libraries or sequences that encode an engineered diversity of amino acids in loop regions of alternative non-antibody scaffolds, such as fibrinogen domains (see, e.g., Weisel et al. (1985) Science 230:1388), Kunitz domains (see, e.g., U.S. Pat. No. 6,423,498), lipocalin domains (see, e.g., WO 2006/095164), V-like domains (see, e.g., US Patent Application Publication No. 2007/0065431), C-type lectin domains (Zelensky and Gready (2005) FEBS J. 272:6179), mAb² or Fcab™ (see, e.g., PCT Patent Application Publication Nos. WO 2007/098934; WO 2006/072620), or the like. For example, binding domains of this disclosure may be identified by screening a Fab phage library for Fab fragments that specifically bind to a CD3 chain (see Hoet et al. (2005) Nature Biotechnol. 23:344).

Additionally, traditional strategies for hybridoma development using a CD3 chain as an immunogen in convenient systems (e.g., mice, HuMAb Mouse®, TC Mouse™, KM-Mouse®, llamas, chicken, rats, hamsters, rabbits, etc.) can be used to develop binding domains of this disclosure.

In some embodiments, a binding domain is a single chain Fv fragment (scFv) that comprises V_{H} and V_{L} domains specific for a TCR complex or a component thereof. In preferred embodiments, the V_{H} and V_{L} domains are human or humanized V_{H} and V_{L} domains. Exemplary V_{H} domains include BC3 V_{H}, OKT3 V_{H}, H57 V_{H}, and 2C11 V_{H} domains as set forth in SEQ ID NOS:2, 6, 49 and 58, respectively. Further exemplary V_{H} domains include Cris-7 V_{H} domains, such as those set forth in SEQ ID NOS:220, 243, 244, and 245. Exemplary V_{L} domains are BC3 V_{L}, OKT3 V_{L}, H57 V_{L}, and 2C11 V_{L} domains as set forth in SEQ ID NOS:4, 8, 51 and 60, respectively. Further exemplary V_{L} domains include Cris-7 V_{L} domains, such as those set forth in SEQ ID NOS:222, 241, and 242. In certain embodiments, a binding domain comprises or is a sequence that is at least 90%, at least 91%, at least 92%, at least 93%, at least 94%, at least 95%, at least 96%, at least 97%, at least 98%, at least 99%, at least 99.5%, or 100% identical to an amino acid sequence of a light chain variable region (V_{L}) (e.g., SEQ ID NO:4, 8, 51, 60, 222, 241, or 242) or to a heavy chain variable region (V_{H}) (e.g., SEQ ID NO:2, 6, 49, 58, 220, 243, 244, or 245), or both from a monoclonal antibody or fragment or derivative thereof that specifically binds to a TCR complex or a component thereof, such as CD3ε, TCRα, TCRβ, TCRγ and TCRδ, or a combination thereof.

“Sequence identity,” as used herein, refers to the percentage of amino acid residues in one sequence that are identical with the amino acid residues in another reference polypeptide sequence after aligning the sequences and introducing gaps, if necessary, to achieve the maximum percent sequence identity, and not considering any conservative substitutions as part of the sequence identity. The percentage sequence identity values can be generated using the NCBI BLAST2.0 software as defined by Altschul et al. (1997) “Gapped BLAST and PSI-BLAST: a new generation of protein database search programs”, Nucleic Acids Res. 25:3389-3402, with the parameters set to default values.

In certain embodiments, a binding domain V_{H} region of the present disclosure can be derived from or based on a V_{H} of a known monoclonal antibody (e.g., Cris-7, BC3, OKT3, including derivatives thereof) and contains one or more insertions, one or more deletions, one or more amino acid substitutions (e.g., conservative amino acid substitutions or non-conservative amino acid substitutions), or a combination of the above-noted changes, when compared with the V_{H} of a known monoclonal antibody. The insertion(s), deletion(s) or substitution(s) may be anywhere in the V_{H} region, including at the amino- or carboxy-terminus or both ends of this region, provided a binding domain containing the modified V_{H} region can still specifically bind its target with an affinity similar to the wild type binding domain.

In certain embodiments, a V_{L} region in a binding domain of the present disclosure is derived from or based on a V_{L} of a known monoclonal antibody (e.g., Cris-7, BC3, OKT3, including derivatives thereof) and contains one or more insertions, one or more deletions, one or more amino acid substitutions (e.g., conservative amino acid substitutions), or a combination of the above-noted changes, when compared with the V_{L} of the known monoclonal antibody. The insertion(s), deletion(s) or substitution(s) may be anywhere in the V_{L} region, including at the amino- or carboxy-terminus or both ends of this region, provided a binding domain containing the modified V_{L} region can still specifically bind its target with an affinity similar to the wild type binding domain.

The V_{H} and V_{L} domains may be arranged in either orientation (i.e., from amino-terminus to carboxy terminus, V_{H}-V_{L} or V_{L}-V_{H}) and may be separated by a variable domain linking sequence. In certain embodiments, variable domain linking sequences include those belonging to the family of GlySer, Gly₂Ser (SEQ ID NO:339), Gly₃Ser (SEQ ID NO:340), Gly₄Ser (SEQ ID NO:341), and Gly₅Ser (SEQ ID NO:342), including (Gly₃Ser)₁(Gly₄Ser)₁ (SEQ ID NO:343), (Gly₃Ser)₂(Gly₄Ser)₁ (SEQ ID NO:344), (Gly₃Ser)₃(Gly₄Ser)₁ (SEQ ID NO:345), (Gly₃Ser)₄(Gly₄Ser)₁ (SEQ ID NO:346), (Gly₃Ser)₅(Gly₄Ser)₁ (SEQ ID NO:347), (Gly₃Ser)₁(Gly₄Ser)₁ (SEQ ID NO:348), (Gly₃Ser)₁(Gly₄Ser)₂ (SEQ ID NO:349), (Gly₃Ser)₁(Gly₄Ser)₃ (SEQ ID NO:350), (Gly₃Ser)₁(Gly₄Ser)₄ (SEQ ID NO:351), (Gly₃Ser)₁(Gly₄Ser)₅ (SEQ ID NO:352), (Gly₃Ser)₃(Gly₄Ser)₃ (SEQ ID NO:353), (Gly₃Ser)₄(Gly₄Ser)₄ (SEQ ID NO:354), (Gly₃Ser)₅(Gly₄Ser)₅ (SEQ ID NO:355), or (Gly₄Ser)₂ (SEQ ID NO:356), (Gly₄Ser)₃ (SEQ ID NO:145), (Gly₄Ser)₄ (SEQ ID NO:357), or (Gly₄Ser)₅ (SEQ ID NO:358). In certain embodiments, the variable domain linking sequence is GGGGSGGGGSGGGGSAQ (SEQ ID NO:98). In preferred embodiments, these (GlyₓSer)-based linkers are used to link variable domains and are not used to link a binding domain (e.g., scFv) to an Fc tail (e.g., an IgG CH₂CH₃). In certain embodiments, a variable domain linking sequence comprises from about 5 to about 35 amino acids and preferably comprises from about 15 to about 25 amino acids.

Any of the insertion(s), deletion(s) or substitution(s) at the amino- or carboxy-terminus of a particular domain or region, as described herein, may be a result, for example, of how one variable region is engineered to be linked to another variable region (e.g., amino acid changes at the junctions between a V_{H} and a V_{L} region, or between a V_{L} and a V_{H} region) or how a binding domain is engineered to be linked to a constant region (e.g., amino acid changes at the junction between a binding domain and a hinge linker). For example, one or more (e.g., 2-8) amino acids may be added, deleted, or substituted at one or more of the fusion protein junctions, as described in more detail below.

Exemplary binding domains of the present disclosure include those as set forth in SEQ ID NOS:18, 20, 48, 62, and 258-264. In certain preferred embodiments, a single chain fusion protein of this disclosure comprises a binding domain having an amino acid sequence as set forth in any one of SEQ ID NOS:258-264.

### Linker Polypeptide

As described herein, fusion proteins of the present disclosure comprise a linker polypeptide that links a binding domain that specifically binds to a TCR complex or component thereof to either an immunoglobulin C_{H2} region or an immunoglobulin C_{H3} region. In addition to providing a spacing function between the binding domain and the rest of a fusion protein, a linker can provide flexibility or rigidity suitable for properly orienting the binding domain of a fusion protein to interact with its target (i.e., a TCR complex or a component thereof, such as CD3). Further, a linker can support expression of a full-length fusion protein and provide stability for a purified protein both in vitro and in vivo following administration to a subject in need thereof, such as a human, and is preferably non-immunogenic or poorly immunogenic in such a subject.

Linkers contemplated in this disclosure include, for example, peptides derived from an interdomain region of an immunoglobulin superfamily member, an immunoglobulin interdomain region (e.g., an antibody hinge region), or a stalk region of C-type lectins, a family of type II membrane proteins (see, e.g., exemplary lectin stalk region sequences set forth in of PCT Application Publication No. WO 2007/146968, such as SEQ ID NOS:111, 113, 115, 117, 119, 121, 123, 125, 127, 129, 131, 133, 135, 149, 151, 153, 155, 157, 159, 161, 163, 165, 167, 169, 231, 233, 235, 237, 239, 241, 243, 245, 247, 249, 251, 253, 255, 257, 259, 261, 263, 265, 267, 269, 271, 273, 275, 277, 279, 281, 287, 289, 297, 305, 307, 309-311, 313-331, 346, 373-377, 380, or 381 from that publication, which are incorporated herein by reference), and a cluster of differentiation (CD) molecule stalk region.

A linker suitable for use in the fusion proteins of this disclosure includes an antibody hinge region selected from an IgG hinge, IgA hinge, IgD hinge, IgE C_{H2}, IgM C_{H2}, or fragments or variants thereof. In certain preferred embodiments, a linker may be an antibody hinge region selected from human IgG1, human IgG2, human IgG3, human IgG4, or fragments or variants thereof. In some embodiments, the linker is a wild type immunoglobulin hinge region, such as a wild type human immunoglobulin hinge region. Exemplary linkers are a wild type human IgG1 hinge region and a wild type mouse IGHG2c hinge region, the sequence of which are set forth in SEQ ID NOS:63 and 72, respectively.

In certain embodiments, one or more amino acid residues may be added at the amino- or carboxy-terminus of a wild type immunoglobulin hinge region as part of a fusion protein construct design. Representative modified linkers can have additional junction amino acid residues at the amino-terminus, such as “RT” (e.g., shown in SEQ ID NOS:100 and 52), “RSS” (e.g., shown in SEQ ID NOS:328 and 331-338), “TG” (e.g., shown in SEQ ID NO:177), or “T” (e.g., shown in SEQ ID NO:300); at the carboxy-terminus, such as “SG” (e.g., shown in SEQ ID NOS:212 and 213); or a deletion combined with an addition, such as ΔP with “SG” added at the carboxy terminus (e.g., shown in SEQ ID NO:212).

In preferred embodiments, a linker is a mutated immunoglobulin hinge region, such as a mutated IgG immunoglobulin hinge region. For example, a wild type human IgG1 hinge region contains three cysteine residues: The most amino-terminal cysteine is referred to as the first cysteine, whereas the most carboxy-terminal cysteine of the hinge region is referred to as the third cysteine. In certain embodiments, a linker is a mutated human IgG1 hinge region with only two cysteine residues, such as a human IgG1 hinge region with the first cysteine substituted by a serine. In certain other embodiments, a linker is a mutated human IgG1 hinge region with only one cysteine residue, such as the first, second, or third cysteine. In certain embodiments, the first proline carboxy-terminal to the third cysteine in a human IgG1 hinge region is substituted, for example, by a serine. Exemplary mutated human IgG1 hinge regions that may be used as a linker polypeptide between a binding domain and the rest of the fusion protein are listed in the sequence listing, such as linkers 47-49, 51, and 53-60 (SEQ ID NOS:99, 146-148 and 150-157, respectively). In certain embodiments, one or more amino acid residues may be added at the amino- or carboxy-terminus of a mutated immunoglobulin hinge region as part of a fusion protein construct design. Examples of such modified linkers are set forth in SEQ ID NOS:10, 335 and 300, wherein amino acid residues “RT,” “RSS,” or “T”, respectively, are added to the amino-terminus of a mutated human IgG1 hinge region.

In certain embodiments, a linker may have one or more than one cysteine residue but has a single cysteine residue for formation of an interchain disulfide bond, such as the second or third cysteine of IgG1. In other embodiments, a linker may have more than two cysteine residues but has two cysteine residues for formation of interchain disulfide bonds.

In certain embodiments, linker polypeptides of the present disclosure are derived from a wild type immunoglobulin hinge region (e.g., an IgG1 hinge region) and contain one or more (e.g., 1, 2, 3, or 4) insertions, one or more (e.g., 1, 2, 3, or 4) deletions, one or more (e.g., 1, 2, 3, or 4) amino acid substitutions (e.g., conservative amino acid substitutions or non-conservative amino acid substitutions), or a combination of the above-noted mutations, when compared with the wild type immunoglobulin hinge region and provided the modified hinge retains the flexibility or rigidity suitable for properly orienting the binding domain of a fusion protein to interact with its target. The insertion(s), deletion(s) or substitution(s) may be anywhere in the wild type immunoglobulin hinge region, including at the amino- or carboxy-terminus or both ends. In certain embodiments, a linker polypeptide comprises or is a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a wild type immunoglobulin hinge region, such as a wild type human IgG1 hinge, a wild type human IgG2 hinge, or a wild type human IgG4 hinge.

Alternative hinge or linker sequences may be crafted from portions of cell surface receptors that connect IgV-like or IgC-like domains. Regions between IgV-like domains where a cell surface receptor contains multiple IgV-like domains in tandem and between IgC-like domains where a cell surface receptor contains multiple tandem IgC-like regions could also be used as a connecting region or linker peptide. Representative hinge or linker sequences of the interdomain regions between the IgV-like and IgC-like or between the IgC-like or IgV-like domains are found in CD2, CD4, CD22, CD33, CD48, CD58, CD66, CD80, CD86, CD96, CD150, CD166, and CD244. More alternative hinges may be crafted from disulfide-containing regions of Type II receptors from non-immunoglobulin superfamily members, such as CD69, CD72, and CD161.

In certain embodiments, hinge or linker sequences have 2 to 150 amino acid, 5 to 60 amino acids, 2 to 40 amino acids, preferably have 8-20, more preferably have 12-15 amino acids, and may be primarily flexible, but may also provide more rigid characteristics or may contain primarily a helical structure with minimal β sheet structure. Preferably, hinge and linker sequences are stable in plasma and serum and are resistant to proteolytic cleavage. In certain embodiments, the first lysine in the IgG1 upper hinge region is mutated to minimize proteolytic cleavage, preferably the lysine is substituted with methionine, threonine, alanine or glycine, or is deleted (see, e.g., SEQ ID NOS:379-434, which may include junction amino acids at the amino end, preferably RT). In some embodiments, sequences may contain a naturally occurring or added motif such as a core structure CPPC (SEQ ID NO:330) that confers the capacity to form a disulfide bond or multiple disulfide bonds to stabilize the carboxy-terminus of a molecule. In other embodiments, sequences may contain one or more glycosylation sites. An unexpected feature of altering hinge length is allowing modulation of the level of calcium flux caused by single chain fusion proteins of the present disclosure (see, Example 5). Exemplary hinges for modulating calcium flux include SEQ ID NOS:212-218. In addition, hinge length and/or sequence may also affect the activities of fusion proteins in blocking T cell response to alloantigen (see Example 10). Linkers useful as connecting regions in the fusion proteins of this disclosure are set forth in SEQ ID NOS:379-434.

### Immunoglobulin C_{H2} Region Polypeptide

As described herein, a fusion protein of the present disclosure may comprise an immunoglobulin C_{H2} region that comprises an amino acid substitution at the asparagine of position 297 (e.g., asparagine to alanine). Such an amino acid substitution reduces or eliminates glycosylation at this site and abrogates efficient Fc binding to FcγR and C1q.

In certain embodiments, a fusion protein of the present disclosure may comprise an immunoglobulin C_{H2} region that comprises at least one substitution or deletion at positions 234 to 238. For example, an immunoglobulin C_{H2} region can comprise a substitution at position 234, 235, 236, 237 or 238, positions 234 and 235, positions 234 and 236, positions 234 and 237, positions 234 and 238, positions 234-236, positions 234, 235 and 237, positions 234, 236 and 238, positions 234, 235, 237, and 238, positions 236-238, or any other combination of two, three, four, or five amino acids at positions 234-238. In addition or alternatively, a mutated C_{H2} region may comprise one or more (e.g., two, three, four or five) amino acid deletions at positions 234-238, preferably at one of position 236 or position 237 while the other position is substituted. The above-noted mutation(s) decrease or eliminate the antibody-dependent cell-mediated cytotoxicity (ADCC) activity or Fc receptor-binding capability of the fusion protein. In certain preferred embodiments, the amino acid residues at one or more of positions 234-238 has been replaced with one or more alanine residues. In further preferred embodiments, only one of the amino acid residues at positions 234-238 have been deleted while one or more of the remaining amino acids at positions 234-238 can be substituted with another amino acid (e.g., alanine or serine).

In certain other embodiments, a fusion protein of the present disclosure may comprise an immunoglobulin C_{H2} region that comprises one or more amino acid substitutions at positions 253, 310, 318, 320, 322, and 331. For example, an immunoglobulin C_{H2} region can comprise a substitution at position 253, 310, 318, 320, 322, or 331, positions 318 and 320, positions 318 and 322, positions 318, 320 and 322, or any other combination of two, three, four, five or six amino acids at positions 253, 310, 318, 320, 322, and 331. The above-noted mutation(s) decrease or eliminate the complement-dependent cytotoxicity (CDC) of the fusion protein.

In certain other embodiments, in addition to the amino acid substitution at position 297, a mutated C_{H2} region in a fusion protein of the present disclosure can further comprise one or more (e.g., two, three, four, or five) additional substitutions at positions 234-238. For example, an immunoglobulin C_{H2} region can comprise a substitution at positions 234 and 297, positions 234, 235, and 297, positions 234, 236 and 297, positions 234-236 and 297, positions 234, 235, 237 and 297, positions 234, 236, 238 and 297, positions 234, 235, 237, 238 and 297, positions 236-238 and 297, or any combination of two, three, four, or five amino acids at positions 234-238 in addition to position 297. In addition or alternatively, a mutated C_{H2} region may comprise one or more (e.g., two, three, four or five) amino acid deletions at positions 234-238, such as at position 236 or position 237. The additional mutation(s) decreases or eliminates the antibody-dependent cell-mediated cytotoxicity (ADCC) activity or Fc receptor-binding capability of the fusion protein. In certain embodiments, the amino acid residues at one or more of positions 234-238 have been replaced with one or more alanine residues. In further embodiments, only one of the amino acid residues at positions 234-238 has been deleted while one or more of the remaining amino acids at positions 234-238 can be substituted with another amino acid (e.g., preferably alanine or serine).

In certain embodiments, in addition to one or more (e.g., 2, 3, 4, or 5) amino acid substitutions at positions 234-238, the mutated C_{H2} region in a fusion protein of the present disclosure may contain one or more (e.g., 2, 3, 4, 5, or 6) additional amino acid substitutions (e.g., substituted with alanine) at one or more positions involved in complement fixation (e.g., at positions I253, H310, E318, K320, K322, or P331). Preferred mutated immunoglobulin C_{H2} regions include human IgG1, IgG2, IgG4 and mouse IgG2a C_{H2} regions with alanine substitutions at positions 234, 235, 237 (if present), 318, 320 and 322. An exemplary mutated immunoglobulin C_{H2} region is mouse IGHG2c C_{H2} region with alanine substitutions at L234, L235, G237, E318, K320, and K322 (SEQ ID NO:50).

In still further embodiments, in addition to the amino acid substitution at position 297 and the additional deletion(s) or substitution(s) at positions 234-238, a mutated C_{H2} region in a fusion protein of the present disclosure can further comprise one or more (e.g., two, three, four, five, or six) additional substitutions at positions 253, 310, 318, 320, 322, and 331. For example, an immunoglobulin C_{H2} region can comprise a (1) substitution at position 297, (2) one or more substitutions or deletions or a combination thereof at positions 234-238, and one or more (e.g., 2, 3, 4, 5, or 6) amino acid substitutions at positions 1253, H310, E318, K320, K322, and P331, such as one, two, three substitutions at positions E318, K320 and K322. Preferably, the amino acids at the above-noted positions are substituted by alanine or serine.

In certain embodiments, the immunoglobulin C_{H2} region polypeptide comprises: (i) an amino acid substitution at the asparagine of position 297 and one amino acid substitution at position 234, 235, 236 or 237; (ii) an amino acid substitution at the asparagine of position 297 and amino acid substitutions at two of positions 234-237; (iii) an amino acid substitution at the asparagine of position 297 and amino acid substitutions at three of positions 234-237; (iv) an amino acid substitution at the asparagine of position 297, amino acid substitutions at positions 234, 235 and 237, and an amino acid deletion at position 236; (v) amino acid substitutions at three of positions 234-237 and amino acid substitutions at positions 318, 320 and 322; or (vi) amino acid substitutions at three of positions 234-237, an amino acid deletion at position 236, and amino acid substitutions at positions 318, 320 and 322.

Exemplary mutated immunoglobulin C_{H2} regions with amino acid substitutions at the asparagine of position 297 in the fusion proteins of the present disclosure include: human IgG1 C_{H2} region with alanine substitutions at L234, L235, G237 and N297 and a deletion at G236 (SEQ ID NO:103), human IgG2 C_{H2} region with alanine substitutions at V234, G236, and N297 (SEQ ID NO:104), human IgG4 C_{H2} region with alanine substitutions at F234, L235, G237 and N297 and a deletion of G236 (SEQ ID NO:75), human IgG4 C_{H2} region with alanine substitutions at F234 and N297 (SEQ ID NO:375), human IgG4 C_{H2} region with alanine substitutions at L235 and N297 (SEQ ID NO:376), human IgG4 C_{H2} region with alanine substitutions at G236 and N297 (SEQ ID NO:377), and human IgG4 C_{H2} region with alanine substitutions at G237 and N297 (SEQ ID NO:378).

In certain embodiments, in addition to the amino acid substitutions described above, a mutated C_{H2} region in a fusion protein of the present disclosure may contain one or more additional amino acid substitutions at one or more positions other than the above-noted positions. Such amino acid substitutions may be conservative or non-conservative amino acid substitutions. For example, in certain embodiments, P233 may be changed to E233 in a mutated IgG2 C_{H2} region (see, e.g., SEQ ID NO:104). In addition or alternatively, in certain embodiments, the mutated C_{H2} region in a fusion protein of the present disclosure may contain one or more amino acid insertions, deletions, or both. The insertion(s), deletion(s) or substitution(s) may anywhere in an immunoglobulin C_{H2} region, such as at the N- or C-terminus of a wild type immunoglobulin C_{H2} region resulting from linking the C_{H2} region with another region (e.g., a variable region) via a linker.

In certain embodiments, the mutated C_{H2} region in a fusion protein of the present disclosure comprises or is a sequence that is at least 90%, at least 91%, at least 92%, at least 93%, at least 94%, at least 95%, at least 96%, at least 97%, at least 98%, at least 99% identical to a wild type immunoglobulin C_{H2} region, such as the C_{H2} region of wild type human IgG1, IgG2, or IgG4, or mouse IgG2a (e.g., IGHG2c).

A mutated immunoglobulin C_{H2} region in a fusion protein of the present disclosure may be derived from a C_{H2} region of various immunoglobulin isotypes, such as IgG1, IgG2, IgG3, IgG4, IgA1, IgA2, and IgD, from various species (including human, mouse, rat, and other mammals). In certain preferred embodiments, a mutated immunoglobulin C_{H2} region in a fusion protein of the present disclosure may be derived from a C_{H2} region of human IgG1, IgG2 or IgG4, or mouse IgG2a (e.g., IGHG2c), whose sequences are set forth in SEQ ID NOS:64, 66, 68 and 73.

Methods are known in the art for making mutations inside or outside an Fc domain that can alter Fc interactions with Fc receptors (CD16, CD32, CD64, CD89, FcεR1, FcRn) or with the complement component C1q (see, e.g., U.S. Pat. No. 5,624,821; Presta (2002) Curr. Pharma. Biotechnol. 3:237).

In certain embodiments, a fusion protein of the present disclosure does not comprise any immunoglobulin C_{H2} region.

### Immunoglobulin C_{H3} Region Polypeptide

As described herein, a fusion protein of the present disclosure comprises one or more immunoglobulin C_{H3} region polypeptides. In certain embodiments, a fusion protein of the present disclosure does not contain any C_{H2} region. In such embodiments, the binding domain that specifically binds to a TCR complex or a component thereof is directly linked to an immunoglobulin C_{H3} region via a linker (e.g., hinge) polypeptide. In certain embodiments where a C_{H2} region is absent, a fusion protein of the present disclosure may comprise only one C_{H3} region. Alternative embodiments include a fusion protein of the present disclosure that comprises two C_{H3} regions and no C_{H2} region.

In the embodiments where a fusion protein comprises both a mutated immunoglobulin C_{H2} region and an immunoglobulin C_{H3} region, the C_{H2} and C_{H3} regions may be derived from the same, or different, immunoglobulins, antibody isotypes, or allelic variants. Preferably, the C_{H2} region is directly linked to the amino-terminus of the C_{H3} region. Exemplary sequences that comprise a C_{H2} region directly linked to the amino terminus of a C_{H3} region are set forth in SEQ ID NOS:11-14 and 101. Alternatively, the C_{H2} region may be linked to the C_{H3} region via one or more amino acids or via a linker (see, e.g., linkers as set forth in the sequence listing).

In certain embodiments, a fusion protein of the present disclosure may comprise two immunoglobulin C_{H3} regions. These C_{H3} regions may be wild type or mutated C_{H3} regions from the same immunoglobulin isotypes, or may be from different immunoglobulin isotypes. For example, in certain embodiments, a fusion protein comprises a C_{H3} region of human IgM and a C_{H3} region of human IgG1. Exemplary sequences in which a C_{H3} region of human IgM and a C_{H3} region of human IgG1 are linked together include SEQ ID NOS:15 and 74. In certain other embodiments, a fusion protein comprises a mouse C_{H3μ} region and a mouse C_{H3γ} region. Exemplary sequences in which a mouse C_{H3μ} region and a mouse C_{H3γ} region are linked together include SEQ ID NOS:308 and 309.

In the embodiments where the fusion protein comprises two immunoglobulin C_{H3} regions, a C_{H3} region located amino-terminal to the other C_{H3} region is referred to as “the first C_{H3} region.” The other C_{H3} region is referred to as “the second C_{H3} region.” In such embodiments, the two immunoglobulin C_{H3} regions may be fused directly with each other. In other words, the C-terminus of the first C_{H3} region is directly linked to the amino-terminus of the second C_{H3} region without any intervening amino acid residues between them (i.e., in the absence of a linker). Alternatively, the two C_{H3} regions may be linked via one or more (e.g., 2-8) amino acids or via a linker (see, e.g., linkers as set forth in the sequence listing).

In certain embodiments, an immunoglobulin C_{H3} region in the fusion protein of the present disclosure may contain one or more (e.g., 2-8) additional amino acid substitutions. Such amino acid substitutions may be conservative or non-conservative. In addition or alternatively, in certain embodiments, the C_{H3} region in the fusion protein of the present disclosure may contain one or more (e.g., 2-8) amino acid insertions, deletions, or both at different positions. The insertion(s), deletion(s) or substitution(s) may be anywhere in an immunoglobulin C_{H3} region, including at the amino- or carboxy-terminus or both.

In certain embodiments, the immunoglobulin C_{H3} region in the fusion protein of the present disclosure comprises or is a sequence that is at least 90%, at least 91%, at least 92%, at least 93%, at least 94%, at least 95%, at least 96%, at least 97%, at least 98%, at least 99% identical to a wild type immunoglobulin C_{H3} region, such as the C_{H3} region of wild type human IgM, IgG1, IgG2, or IgG4.

In certain embodiments, an immunoglobulin C_{H3} region polypeptide is a wild type immunoglobulin C_{H3} region polypeptide, including a wild type C_{H3} region of any one of the various immunoglobulin isotypes (e.g., IgA, IgD, IgG1, IgG2, IgG3, IgG4, IgE, or IgM) from various species (i.e., human, mouse, rat or other mammals). For example, the immunoglobulin C_{H3} region may be a wild type human IgG1 C_{H3} region (e.g., SEQ ID NO:65), a wild type human IgG2 C_{H3} region (e.g., SEQ ID NO:67), a wild type human IgG4 C_{H3} region (e.g., SEQ ID NO: 69), a wild type human IgM C_{H3} region (e.g., SEQ ID NO:71), a mouse C_{H3μ} region (e.g., SEQ ID NO:329) or a wild type mouse IGHG2c C_{H3} region (e.g., SEQ ID NO:54). In further embodiments, an immunoglobulin C_{H3} region polypeptide is a mutated immunoglobulin C_{H3} region polypeptide. The mutations in the immunoglobulin C_{H3} region may be at one or more positions that are involved in complement fixation, such as at H433 or N434.

### Additional Sequences and Modifications

As described herein, a single chain fusion protein of the present disclosure can comprise from amino-terminus to carboxy-terminus: (a) a binding domain that specifically binds to CD3 (such as CD3ε), (b) a linker polypeptide, (c) optionally an immunoglobulin C_{H2} region polypeptide, and (d) an immunoglobulin C_{H3} region polypeptide. In addition, a fusion protein of the present disclosure may comprise one or more additional regions, such as a leader sequence at its amino-terminus for expression of a fusion protein, an additional Fc sub-region (e.g., a wild type or mutated C_{H4} region of IgM or IgE), or a tail sequence at its carboxy-terminus for identification or purification purposes. Exemplary tail sequence may include epitope tags for detection or purification, such as a 6-Histidine region or a FLAG epitope.

For example, the fusion protein may have additional amino acid residues that arise from use of specific expression systems. For example, use of commercially available vectors that express a desired polypeptide as part of a glutathione-S-transferase (GST) fusion product provides the desired polypeptide having an additional glycine residue at position −1 after cleavage of the GST component from the desired polypeptide. Variants which result from expression in other vector systems are also contemplated, including those wherein histidine tags are incorporated into the amino acid sequence, generally at the carboxy and/or amino terminus of the sequence. An exemplary additional sequence that may be present at the carboxy- or amino-terminus of a fusion protein comprises three copies of the FLAG epitope, one copy of the AVI tag, and six histidines as set forth in SEQ ID NO:70.

In certain embodiments, the fusion protein of the present disclosure comprises a leader peptide at its N-terminus. The lead peptide facilitates secretion of expressed fusion proteins. Using any of the conventional leader peptides (signal sequences) is expected to direct nascently expressed polypeptides or fusion proteins into a secretory pathway and to result in cleavage of the leader peptide from the mature fusion protein at or near the junction between the leader peptide and the fusion protein. A particular leader peptide will be chosen based on considerations known in the art, such as using sequences encoded by nucleic acid molecules that allow the easy inclusion of restriction endonuclease cleavage sites at the beginning or end of the coding sequence for the leader peptide to facilitate molecular engineering, provided that such introduced sequences specify amino acids that either do not interfere unacceptably with any desired processing of the leader peptide from the nascently expressed protein or do not unacceptably interfere with any desired function of a polypeptide or fusion protein if the leader peptide is not cleaved during maturation of the polypeptides or fusion proteins. Exemplary leader peptides of this disclosure include natural leader sequences or others, such as H₃N-MDFQVQIFSFLLISASVIMSRG-CO₂H (SEQ ID NO:9).

In certain embodiments, a fusion protein of the present disclosure is glycosylated, wherein the pattern of glycosylation is dependent upon a variety of factors including the host cell in which the protein is expressed (if prepared in recombinant host cells) and the culture conditions.

In further embodiments, the immunoglobulin C or C_{H3} regions of a fusion protein of the present disclosure may have an altered glycosylation pattern relative to the C_{H2} or C_{H3} regions of an immunoglobulin reference sequence. For example, any of a variety of genetic techniques may be employed to alter one or more particular amino acid residues that form a glycosylation site (see Co et al. (1993) Mol. Immunol. 30:1361; Jacquemon et al. (2006) J. Thromb. Haemost. 4:1047; Schuster et al. (2005) Cancer Res. 65:7934; Warnock et al. (2005) Biotechnol. Bioeng. 92:831). Alternatively, the host cells in which fusion proteins of this disclosure are produced may be engineered to produce an altered glycosylation pattern.

In certain embodiments, the present disclosure also provides derivatives of the fusion proteins described herein. Derivatives include fusion proteins bearing modifications other than insertions, deletions, or substitutions of amino acid residues. Preferably, the modifications are covalent in nature, and include for example, chemical bonding with polymers, lipids, other organic and inorganic moieties. Derivatives of this disclosure may be prepared to increase circulating half-life of a specific fusion protein, or may be designed to improve targeting capacity for the fusion protein to desired cells, tissues, or organs.

In certain embodiments, the in vivo half-life of the fusion protein of this disclosure can be increased using methods known in the art for increasing the half-life of large molecules. For example, this disclosure embraces fusion proteins that are covalently modified or derivatized to include one or more water-soluble polymer attachments, such as polyethylene glycol, polyoxyethylene glycol, or polypropylene glycol (see, e.g., U.S. Pat. Nos. 4,640,835; 4,496,689; 4,301,144; 4,670,417; 4,791,192; 4,179,337). Still other useful polymers known in the art include monomethoxy-polyethylene glycol, dextran, cellulose, and other carbohydrate-based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of these polymers. Particularly preferred are polyethylene glycol (PEG)-derivatized proteins. Water-soluble polymers may be bonded at specific positions, for example at the amino terminus of the fusion proteins according to this disclosure, or randomly attached to one or more side chains of the polypeptide. The use of PEG for improving therapeutic capacities is described in U.S. Pat. No. 6,133,426.

In some embodiments, a fusion protein according to the present disclosure is a PIMS molecule that further contains an amino-terminally disposed immunoglobulin hinge region. The amino-terminal hinge region may be the same as, or different than, the linker found between an immunoglobulin C_{H3} region and a binding domain. In some embodiments, an amino-terminally disposed linker contains a naturally occurring or added motif (such as CPPC, SEQ ID NO:330) to promote the formation of at least one disulfide bond to stabilize the amino-terminus of a dimerized or multimerized molecule.

### Methods for Making and Purifying Fusion Proteins

The fusion proteins of the present disclosure may be made according to methods known in the art. For example, methods for making SMIP fusion proteins are described in U.S. Patent Publication Nos. 2003/0133939, 2003/0118592 and 2005/0136049, and methods for making PIMS proteins are described, for example, PCT Application Publication No. WO 2009/023386.

In certain embodiments, the present disclosure provides purified fusion proteins as described herein. The term “purified,” as used herein, refers to a composition, isolatable from other components, wherein the fusion protein is purified to any degree relative to its naturally obtainable state. A “purified protein” therefore also refers to such protein, isolated from the environment in which it naturally occurs. In certain embodiments, the present disclosure provides substantially purified fusion proteins as described herein. “Substantially purified” refers to a protein composition in which the protein forms the major component of the composition, such as constituting at least about 50%, such as at least about 60%, about 70%, about 80%, about 90%, about 95%, about 99%, of the protein, by weight, in the composition.

Protein purification techniques are well known to those of skill in the art. These techniques involve, at one level, the crude fractionation of the polypeptide and non-polypeptide fractions. Further purification using chromatographic and electrophoretic techniques to achieve partial or complete purification (or purification to homogeneity) is frequently desired. Analytical methods particularly suited to the preparation of a pure fusion protein are ion-exchange chromatography, exclusion chromatography; polyacrylamide gel electrophoresis; and isoelectric focusing. Particularly efficient methods of purifying peptides are fast protein liquid chromatography and HPLC.

Various methods for quantifying the degree of purification are known to those of skill in the art in light of the present disclosure. These include, for example, determining the specific binding activity of an active fraction, or assessing the amount of protein in a fraction by SDS/PAGE analysis. A preferred method for assessing the purity of a protein fraction is to calculate the binding activity of the fraction, to compare it to the binding activity of the initial extract, and to thus calculate the degree of purification, herein assessed by a “-fold purification number.” The actual units used to represent the amount of binding activity will, of course, be dependent upon the particular assay technique chosen to follow the purification and whether or not the expressed protein exhibits a detectable binding activity.

### Exemplary Fusion Proteins

Exemplary single chain fusion proteins of the present disclosure include BC3 IgG1 N297, BC3 IgG1AA, BC3 IgG2AA, BC3 IgG4AA, BC3 HM1, BC3 ΔC_{H2}, OKT3 IgG1AA, OKT3 IgG2AA, OKT3 IgG4AA, OKT3 HM1, OKT3 ΔC_{H2}, H57 null2, and 2C11 null2 as set forth in SEQ ID NOS:80-85, 88-93, 96 and 97, respectively. Exemplary preferred single chain fusion proteins of the present disclosure include chimeric Cris-7 IgG1AA, chimeric Cris-7 IgG2AA, chimeric Cris-7 IgG4AA, chimeric Cris-7 HM1, humanized Cris-7 IgG1AA, humanized Cris-7 IgG2AA, humanized Cris-7 IgG4AA, and humanized Cris-7 HM1, as set forth in SEQ ID NOS:265-299, respectively. Additional exemplary single chain fusion proteins include BC3 HM1, BC3 ΔC_{H2}, OKT3 HM1, and OKT3 ΔC_{H2} without their carboxy-terminal tags as set forth in SEQ ID NOS:86, 87, 94, and 95, respectively. Further exemplary fusion proteins include the above-noted fusion protein with their leader sequences at the amino-terminus as set forth in SEQ ID NOS:22, 24, 26, 28, 30, 32, 34, 36, 38, 40, 42, 47, 56, 76-79, 224, 226, 228, 230, 232, 234, 236, 238, 240, 247, 249, 251, 253, 255, and 257. Additional exemplary fusion proteins with their leader sequences at the amino-terminus include H57 half null (SEQ ID NO:304) and H57 HM2 (SEQ ID NO:306). Further exemplary fusion proteins are BC3 IgG1 N297 with various linker sequences as set forth in SEQ ID NOS:311, 313, 315, 317, 319, 321, 323, 325 and 327. Several of these exemplary single chain fusion proteins are described in detail in the Examples section below.

### Functional Features

As described herein, a single chain fusion protein of the present disclosure may have one or more (e.g., 2, 3, 4, 5, 6, 7), or any combination thereof, of the following characteristics or functional features: (1) not activating T cells, (2) not inducing or inducing minimal cytokine release, (3) inducing phosphorylation of molecules in the TCR signaling pathway, (4) increasing calcium flux more than the corresponding monoclonal antibody, (5) blocking T cell response to an alloantigen, (6) blocking memory T cell response to an antigen, and (7) downmodulating the TCR complex.

In certain preferred embodiments, a single chain fusion protein of the present disclosure does not or minimally activates T cells. A fusion protein “does not or minimally or nominally activates T cells” if, when used to treat T cells (e.g., PHA- or ConA-primed T cells), the fusion protein does not cause a statistically significant increase in the percentage of activated T cells as compared to untreated cells in at least one in vitro or in vivo assay provided in the examples of the present disclosure. Preferably, T cell activation is measured in the in vitro primed T cell activation assay described in Example 1.

In further preferred embodiments, a fusion protein of the present disclosure does not induce a cytokine storm or does not induce a clinically relevant cytokine release. A fusion protein “does not induce a cytokine storm” (also referred to as “inducing an undetectable, nominal, or minimal cytokine release” or “does not induce or induces a minimally detectable cytokine release”) if, when used to treat T cells, it does not cause a statistically significant increase in the amount of at least one cytokine including IFNγ; preferably at least two cytokines including IFNγ and TNFα or IL-6 and TNFα; preferably three cytokines including IL-6, IFNγ, and TNFα; preferably four cytokines including IL-2, IL-6, IFNγ, and TNFα; and preferably at least five cytokines including IL-2, IL-6, IL-10, IFNγ, and TNFα; released from treated cells as compared to no treatment in at least one in vitro or in vivo assay known in the art or provided in the examples of the present disclosure. Preferably the cytokine storm is measured in the in vitro cytokine release by primed T cells assay described in Example 1. Clinically, cytokine-release syndrome is characterized by fever, chills, rash, nausea, and sometimes dyspnea and tachycardia, which is in parallel with maximal release of certain cytokines, such as IFNγ, as well as IL-2, IL-6, and TNFα. Cytokines that may be tested for release in an in vitro assay or in vivo include G-CSF, GM-CSF, IL-2, IL-4, IL-5, IL-6, IL-10, IL-13, IL-17, IP-10, KC, MCPJ, IFNγ, and TNFα; and more preferably include IL-2, IL-6, IL-10, IFNγ, and TNFα.

In further preferred embodiments, a fusion protein of the present disclosure causes an increase in calcium flux in cells, such as T cells. A fusion protein causes an “increase in calcium” if, when used to treat T cells, it causes a statistically significant, rapid increase in calcium flux of the treated cells (preferably within 300 seconds, more preferably within 200 seconds, and most preferably within 100 seconds of treatment) as compared to cells treated with the corresponding antibody (i.e., an antibody with the same binding domain as a single chain fusion protein of this disclosure) in an in vitro assay known in the art or provided herein. Preferably the calcium flux caused by a single chain fusion protein of this disclosure is compared to the flux caused by a corresponding antibody in the in vitro calcium flux assay described in Example 5 and is observed or measured within at least the first 100 to 300 seconds of treatment.

In further embodiments, a single chain fusion protein of the present disclosure induces phosphorylation of a molecule in the TCR signal transduction pathway. The “TCR signal transduction pathway” refers to the signal transduction pathway initiated via the binding of a peptide:MHC ligand to the TCR and its co-receptor (CD4 or CD8). A “molecule in the TCR signal transduction pathway” refers to a molecule that is directly involved in the TCR signal transduction pathway, such as a molecule whose phosphorylation state (e.g., whether the molecule is phosphorylated or not), whose binding affinity to another molecule, or whose enzymatic activity, has been changed in response to the signal from the binding of a peptide:MHC ligand to the TCR and its co-receptor. Exemplary molecules in the TCR signal transduction pathway include the TCR complex or its components (e.g., CD3ζ chains), ZAP-70, Fyn, Lck, phospholipase c-γ, protein kinase C, transcription factor NFκB, phasphatase calcineurin, transcription factor NFAT, guanine nucleotide exchange factor (GEF), Ras, MAP kinase kinase kinase (MAPKKK), MAP kinase kinase (MAPKK), MAP kinase (ERK1/2), and Fos.

A single chain fusion protein of this disclosure “induces phosphorylation of a molecule in the TCR signal transduction pathway” if, when used to treat T cells, it causes a statistically significant increase in phosphorylation of a molecule in the TCR signal transduction pathway (e.g., CD3ζ chains, ZAP-70, and ERK½) in an in vitro or in vivo assay as described in the examples of the present disclosure or receptor signaling assays known in the art. Results from most receptor signaling assays known in the art are determined using immunohistochemical methods, such as western blots or fluorescence microscopy.

In further embodiments, a single chain fusion protein of the present disclosure can block a T cell response to an alloantigen. An “alloantigen” is an antigen existing in alternative (allelic) forms in a species, thus inducing an immune response when a form is transferred to another member of the species who lacks the alloantigen. Exemplary alloantigens can be found, for example, on blood cells (i.e., blood group antigens) or on tissue grafts (i.e., allografts).

A single chain fusion protein of this disclosure “blocks T cell response to an alloantigen” if, when used to treat T cells, it causes a statistically significant decrease in the percentage of T cells activated in response to an alloantigen in an in vitro or in vivo assay, such as the human mixed lymphocyte reaction (MLR) assay and the acute graft versus host disease (aGVHD) model provided in the examples of the present disclosure. Other assays known in the art such as binding assays and skin tests, like footpad swelling assays in mice, which detect delayed type hypersensitivity responses, may also be used to determine reactivity to alloantigen.

In further embodiments, a fusion protein of the present disclosure blocks memory T cell response to an antigen. A single chain fusion protein “blocks memory T cell response to an antigen” if, when used to treat memory T cells, it causes a statistically significant decrease in the percentage of T cells activated in response to a specific antigen (e.g., tetanus toxoid) in an in vitro or in vivo assay, such as the assay analyzing memory T cell activation using tetanus toxoid provided in the examples of the present disclosure. Animal immunization models may also be used to detect a secondary antigen-specific T cell response both in vivo and ex vivo through antigen presentation assays. In addition to the delayed type hypersensitivity assays described above, cytotoxicity assays such as ⁵¹Cr-release assays may be utilized to detect T cell activity (Lavie et al., (2000) International Immunology 12(4):479-486).

In further embodiments, a fusion protein of the present disclosure downmodulates a TCR complex from the surface of a T cell. A single chain fusion protein “downmodulates TCR complex” if, when used to treat T cells, it causes a statistically significant reduction in the number of TCR complexes on the surface of a T cell population in an in vitro or in vivo assay. Useful in vitro or in vivo assays include the assay for evaluating TCR and CD3 downmodulation from the T cell surface provided in the examples of the present disclosure. Such assays compare the amount of cell surface expressed TCR or CD3 prior to and following stimulation as measured by techniques known in the art, such as flow cytometry and immunofluorescence microscopy.

### Methods for Detecting T Cell Activation or Cytokine Release

In a related aspect, the present disclosure provides a method for detecting T cell activation induced by a protein that comprises a binding domain that specifically bindings to a TCR complex or a component thereof, comprising: (a) providing mitogen-primed T cells, (b) treating the primed T cells of step (a) with the protein that comprises a binding domain that specifically binds to a TCR complex or a component thereof, and (c) detecting activation of the primed T cells that have been treated in step (b).

The term “mitogen” as used herein refers to a chemical substance that induces mitosis in lymphocytes of different specificities or clonal origins. Exemplary mitogens that may be used to prime T cells include phytohaemagglutinin (PHA), concanavalin A (ConA), lipopolysaccharide (LPS), pokeweed mitogen (PWM), and phorbol myristate acetate (PMA).

In certain embodiments of methods for detecting T cell activation provided herein, the protein that comprises a binding domain that specifically binds to a TCR complex or a component thereof is a fusion protein provided herein. In certain other embodiments, the protein that comprises a binding domain that specifically binds to a TCR complex or a component thereof is a monoclonal antibody.

T cell activation may be detected by measuring the expression of activation markers known in the art, such as CD25, CD40 ligand, and CD69. Activated T cells may also be detected by cell proliferation assays, such as CFSE labeling and thymidine uptake assays (Adams (1969) Exp. Cell Res. 56:55).

In a related aspect, the present disclosure provides a method for detecting cytokine release induced by a protein that comprises a binding domain that specifically binds to a TCR complex or a component thereof, comprising: (a) providing mitogen-primed T cells, (b) treating the primed T cells of step (a) with the protein that comprises a binding domain that specifically binds to a TCR complex or a component thereof, and (c) detecting release of a cytokine from the primed T cells that have been treated in step (b).

In certain embodiments of methods for detecting cytokine release provided herein, the protein that comprises a binding domain that specifically binds to a TCR complex or a component thereof is a fusion protein provided herein. In certain other embodiments, the protein that comprises a binding domain that specifically binds to a TCR complex or a component thereof is a monoclonal antibody.

### Polynucleotides, Expression Vectors, and Host Cells

This disclosure provides polynucleotides (isolated or purified or pure polynucleotides) encoding the fusion proteins of this disclosure, vectors (including cloning vectors and expression vectors) comprising such polynucleotides, and cells (e.g., host cells) transformed or transfected with a polynucleotide or vector according to this disclosure.

In certain embodiments, a polynucleotide (DNA or RNA) encoding a fusion protein of the present disclosure is contemplated. Exemplary polynucleotides include SEQ ID NOS:21, 23, 25, 27, 29, 31, 33, 35, 37, 39, 41, 43, 46, 55, 303, 306, 310, 312, 314, 316, 318, 320, 322, 324 and 326.

The present invention also relates to vectors that include a polynucleotide of this disclosure and, in particular, to recombinant expression constructs. In one embodiment, this disclosure contemplates a vector comprising a polynucleotide encoding a fusion protein of this disclosure, along with other polynucleotide sequences that can cause or facilitate transcription, translation, and processing of the fusion protein.

Appropriate cloning and expression vectors for use with prokaryotic and eukaryotic hosts are described, for example, in Sambrook et al., Molecular Cloning: A Laboratory Manual, Second Edition, Cold Spring Harbor, N.Y., (1989). Exemplary cloning/expression vectors include cloning vectors, shuttle vectors, and expression constructs, that may be based on plasmids, phagemids, phasmids, cosmids, viruses, artificial chromosomes, or any nucleic acid vehicle known in the art suitable for amplification, transfer, and/or expression of a polynucleotide contained therein

As used herein, “vector” means a nucleic acid molecule capable of transporting another nucleic acid to which it has been linked. Exemplary vectors include plasmids, yeast artificial chromosomes, and viral genomes. Certain vectors can autonomously replicate in a host cell, while other vectors can be integrated into the genome of a host cell and thereby are replicated with the host genome. In addition, certain vectors are referred to herein as “recombinant expression vectors” (or simply, “expression vectors”), which contain nucleic acid sequences that are operatively linked to an expression control sequence and, therefore, are capable of directing the expression of those sequences.

In certain embodiments, expression constructs are derived from plasmid vectors. Illustrative constructs include modified pNASS vector (Clontech, Palo Alto, Calif.), which has nucleic acid sequences encoding an ampicillin resistance gene, a polyadenylation signal and a T7 promoter site; pDEF38 and pNEF38 (CMC ICOS Biologics, Inc.), which have a CHEF1 promoter; and pEE12.4 (Lonza), which has a CMV promoter. Other suitable mammalian expression vectors are well known (see, e.g., Ausubel et al., 1995; Sambrook et al., supra; see also, e.g., catalogs from Invitrogen, San Diego, Calif.; Novagen, Madison, Wis.; Pharmacia, Piscataway, N.J.). Useful constructs may be prepared that include a dihydrofolate reductase (DHFR)-encoding sequence under suitable regulatory control, for promoting enhanced production levels of the fusion proteins, which levels result from gene amplification following application of an appropriate selection agent (e.g., methotrexate).

Generally, recombinant expression vectors will include origins of replication and selectable markers permitting transformation of the host cell, and a promoter derived from a highly-expressed gene to direct transcription of a downstream structural sequence, as described above. A vector in operable linkage with a polynucleotide according to this disclosure yields a cloning or expression construct. Exemplary cloning/expression constructs contain at least one expression control element, e.g., a promoter, operably linked to a polynucleotide of this disclosure. Additional expression control elements, such as enhancers, factor-specific binding sites, terminators, and ribosome binding sites are also contemplated in the vectors and cloning/expression constructs according to this disclosure. The heterologous structural sequence of the polynucleotide according to this disclosure is assembled in appropriate phase with translation initiation and termination sequences. Thus, for example, the fusion protein-encoding nucleic acids as provided herein may be included in any one of a variety of expression vector constructs as a recombinant expression construct for expressing such a protein in a host cell.

The appropriate DNA sequence(s) may be inserted into a vector, for example, by a variety of procedures. In general, a DNA sequence is inserted into an appropriate restriction endonuclease cleavage site(s) by procedures known in the art. Standard techniques for cloning, DNA isolation, amplification and purification, for enzymatic reactions involving DNA ligase, DNA polymerase, restriction endonucleases and the like, and various separation techniques are contemplated. A number of standard techniques are described, for example, in Ausubel et al. (1993 Current Protocols in Molecular Biology, Greene Publ. Assoc. Inc. & John Wiley & Sons, Inc., Boston, Mass.); Sambrook et al. (1989 Molecular Cloning, Second Ed., Cold Spring Harbor Laboratory, Plainview, N.Y.); Maniatis et al. (1982 Molecular Cloning, Cold Spring Harbor Laboratory, Plainview, N.Y.); Glover (Ed.) (1985 DNA Cloning Vol. I and II, IRL Press, Oxford, UK); Hames and Higgins (Eds.), (1985 Nucleic Acid Hybridization, IRL Press, Oxford, UK); and elsewhere.

The DNA sequence in the expression vector is operatively linked to at least one appropriate expression control sequence (e.g., a constitutive promoter or a regulated promoter) to direct mRNA synthesis. Representative examples of such expression control sequences include promoters of eukaryotic cells or their viruses, as described above. Promoter regions can be selected from any desired gene using CAT (chloramphenicol transferase) vectors or other vectors with selectable markers. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art, and preparation of certain particularly preferred recombinant expression constructs comprising at least one promoter or regulated promoter operably linked to a nucleic acid encoding a protein or polypeptide according to this disclosure is described herein.

Variants of the polynucleotides of this disclosure are also contemplated. Variant polynucleotides are at least 90%, and preferably 95%, 99%, or 99.9% identical to one of the polynucleotides of defined sequence as described herein, or that hybridizes to one of those polynucleotides of defined sequence under stringent hybridization conditions of 0.015 M sodium chloride, 0.0015 M sodium citrate at about 65-68° C. or 0.015 M sodium chloride, 0.0015 M sodium citrate, and 50% formamide at about 42° C. The polynucleotide variants retain the capacity to encode a binding domain or fusion protein thereof having the functionality described herein.

The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing are 0.015M sodium chloride, 0.0015M sodium citrate at about 65-68° C. or 0.015M sodium chloride, 0.0015M sodium citrate, and 50% formamide at about 42° C. (see Sambrook et al., Molecular Cloning: A Laboratory Manual, 2nd Ed., Cold Spring Harbor Laboratory, Cold Spring Harbor, N.Y. 1989).

More stringent conditions (such as higher temperature, lower ionic strength, higher concentration of formamide or another denaturing agent) may also be used; however, the rate of hybridization will be affected.

In certain embodiments, less stringent conditions (such as lower temperature, higher ionic strength, lower concentration of formamide or another denaturing agent) may be used. Exemplary less stringent conditions for hydridization and washing are 0.015M sodium chloride, 0.0015M sodium citrate at about 42° C.). The polynucleotide variants retain the capacity to encode a binding domain or fusion protein thereof having the functionality described herein.

A further aspect of this disclosure provides a host cell transformed or transfected with, or otherwise containing, any of the polynucleotides or vector/expression constructs of this disclosure. The polynucleotides or cloning/expression constructs of this disclosure are introduced into suitable cells using any method known in the art, including transformation, transfection and transduction. Host cells include the cells of a subject undergoing ex vivo cell therapy including, for example, ex vivo gene therapy. Eukaryotic host cells contemplated as an aspect of this disclosure when harboring a polynucleotide, vector, or protein according to this disclosure include, in addition to a subject's own cells (e.g., a human patient's own cells), VERO cells, HeLa cells, Chinese hamster ovary (CHO) cell lines (including modified CHO cells capable of modifying the glycosylation pattern of expressed multivalent binding molecules, see US Patent Application Publication No. 2003/0115614), COS cells (such as COS-7), W138, BHK, HepG2, 3T3, RIN, MDCK, A549, PC12, K562, HEK293 cells, HepG2 cells, N cells, 3T3 cells, Spodoptera frugiperda cells (e.g., Sf9 cells), Saccharomyces cerevisiae cells, and any other eukaryotic cell known in the art to be useful in expressing, and optionally isolating, a protein or peptide according to this disclosure. Also contemplated are prokaryotic cells, including Escherichia coli, Bacillus subtilis, Salmonella typhimurium, a Streptomycete, or any prokaryotic cell known in the art to be suitable for expressing, and optionally isolating, a protein or peptide according to this disclosure. In isolating protein or peptide from prokaryotic cells, in particular, it is contemplated that techniques known in the art for extracting protein from inclusion bodies may be used. The selection of an appropriate host is within the scope of those skilled in the art from the teachings herein. Host cells that glycosylate the fusion proteins of this disclosure are contemplated.

The term “recombinant host cell” (or simply “host cell”) refers to a cell containing a recombinant expression vector. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

Recombinant host cells can be cultured in a conventional nutrient medium modified as appropriate for activating promoters, selecting transformants, or amplifying particular genes. The culture conditions for particular host cells selected for expression, such as temperature, pH and the like, will be readily apparent to the ordinarily skilled artisan. Various mammalian cell culture systems can also be employed to express recombinant protein. Examples of mammalian expression systems include the COS-7 lines of monkey kidney fibroblasts, described by Gluzman (1981) Cell 23:175, and other cell lines capable of expressing a compatible vector, for example, the C127, 3T3, CHO, HeLa and BHK cell lines. Mammalian expression vectors will comprise an origin of replication, a suitable promoter and, optionally, enhancer, and also any necessary ribosome binding sites, polyadenylation site, splice donor and acceptor sites, transcriptional termination sequences, and 5′-flanking nontranscribed sequences, for example, as described herein regarding the preparation of multivalent binding protein expression constructs. DNA sequences derived from the SV40 splice, and polyadenylation sites may be used to provide the required nontranscribed genetic elements. Introduction of the construct into the host cell can be effected by a variety of methods with which those skilled in the art will be familiar, including calcium phosphate transfection, DEAE-Dextran-mediated transfection, or electroporation (Davis et al. (1986) Basic Methods in Molecular Biology).

In one embodiment, a host cell is transduced by a recombinant viral construct directing the expression of a protein or polypeptide according to this disclosure. The transduced host cell produces viral particles containing expressed protein or polypeptide derived from portions of a host cell membrane incorporated by the viral particles during viral budding.

### Compositions and Methods of Use

In addition to fusion proteins directed against a TCR complex or a component thereof, the present disclosure also provides pharmaceutical compositions and unit dose forms that comprise the fusion proteins, as well as methods for using the fusion proteins, the pharmaceutical compositions and unit dose forms.

To treat human or non-human mammals suffering a disease state or a condition associated with TCR signaling, a fusion protein is administered to the subject in an amount that is effective to ameliorate symptoms of the disease state or condition following a course of one or more administrations. Being polypeptides, the proteins of this disclosure can be suspended or dissolved in a pharmaceutically acceptable diluent, optionally including a stabilizer or other pharmaceutically acceptable excipient, which can be used for intravenous administration by injection or infusion, as more fully discussed below.

A pharmaceutically effective amount or dose is the amount or dose required to prevent, inhibit the occurrence of, or treat (alleviate a symptom to some extent, preferably all symptoms of) a disease state or condition. In a preferred embodiment, a pharmaceutically effective amount of the single chain fusion proteins of the instant disclosure are used to treat T cell mediated diseases. The pharmaceutically effective dose depends on the type of disease, the composition used, the route of administration, the type of subject being treated, the physical characteristics of the specific subject under consideration for treatment, concurrent medication, and other factors that those skilled in the medical arts will recognize. For example, an amount between 0.1 mg/kg and 100 mg/kg body weight (which can be administered as a single dose, daily, weekly, monthly, or at any appropriate interval) of active ingredient may be administered depending on the potency of a fusion protein of this disclosure.

As described above and illustrated in the examples, fusion proteins directed against a TCR complex or a component thereof, such as CD3, provided herein uniquely engage the TCR signaling pathway without the induction of T cell mitogenicity. Previous studies have demonstrated that peripheral T cell function and differentiation can be driven by manipulation of TCR-associated signaling cascades. For example, both T cell anergy and adaptive regulatory T cells can be induced by strong, non-activating signals. In addition, certain subsets of T cells may be more prone to cell death upon delivery of a strong TCR signal. Thus, the fusion proteins provided herein could be used for the modulation of T cell function and fate, thereby providing therapeutic treatment of T cell mediated disease, including autoimmune or inflammatory diseases in which T cells are significant contributors. Moreover, because the fusion proteins of the present disclosure do not activate T cells and/or do not induce cytokine release, they are advantageous over other molecules directed against the TCR complex (e.g., anti-CD3 antibodies) for having no or reduced side effects such as cytokine release syndrome and acute toxicity.

Exemplary autoimmune or inflammatory disorders (AIID) that may be treated by the fusion proteins and compositions and unit dose forms thereof include, and are not limited to, inflammatory bowel disease (e.g., Crohn's disease or ulcerative colitis), diabetes mellitus (e.g., type I diabetes), dermatomyositis, polymyositis, pernicious anaemia, primary biliary cirrhosis, acute disseminated encephalomyelitis (ADEM), Addison's disease, ankylosing spondylitis, antiphospholipid antibody syndrome (APS), autoimmune hepatitis, Goodpasture's syndrome, Graves' disease, Guillain-Barré syndrome (GBS), Hashimoto's disease, idiopathic thrombocytopenic purpura, systemic lupus erythematosus, lupus nephritis, neuropsychiatric lupus, multiple sclerosis (MS), myasthenia gravis, pemphigus vulgaris, asthma, psoriatic arthritis, rheumatoid arthritis, Sjögren's syndrome, temporal arteritis (also known as “giant cell arteritis”), autoimmune hemolytic anemia, Bullous pemphigoid, vasculitis, coeliac disease, chronic obstructive pulmonary disease, endometriosis, Hidradenitis suppurativa, interstitial cystitis, morphea, scleroderma, narcolepsy, neuromyotonia, vitiligo, and autoimmune inner ear disease.

In certain embodiments, fusion proteins and compositions and unit dose forms provided herein may be used as immunosuppressants with no side effects, or minimal or reduced side effects, associated with cytokine release. For example, single chain fusion proteins and compositions and unit dose forms provided herein may be used in both induction and prevention (i.e., reduce the risk of) or reduction in acute rejection, delayed graft function, and graft loss of solid organ transplants (e.g., kidney, liver, lung, heart transplants). In addition, without inducing T cell activation, in certain embodiments, single chain fusion proteins of this disclosure may be more effective as an immunosuppressant than other molecules directed against the TCR complex known to be both immunosuppressive and T cell mitogenic. In further embodiments, fusion proteins and compositions and unit dose forms provided herein may be used to treat other T cell mediated diseases, such as graft versus host disease (GVHD) and autoimmune and inflammatory disorders (AIID).

In another aspect, compositions of fusion proteins are provided in this disclosure. Pharmaceutical compositions of this disclosure generally comprise a fusion protein provided herein in combination with a pharmaceutically acceptable carrier, excipient, or diluent. Such carriers will be nontoxic to recipients at the dosages and concentrations employed. Pharmaceutically acceptable carriers for therapeutic use are well known in the pharmaceutical art, and are described, for example, in Remington's Pharmaceutical Sciences, Mack Publishing Co. (A. R. Gennaro (Ed.) 1985). For example, sterile saline and phosphate buffered saline at physiological pH may be used. Preservatives, stabilizers, dyes and the like may be provided in the pharmaceutical composition. For example, sodium benzoate, sorbic acid, or esters of p-hydroxybenzoic acid may be added as preservatives. Id. at 1449. In addition, antioxidants and suspending agents may be used. Id. The compounds of the present invention may be used in either the free base or salt forms, with both forms being considered as being within the scope of the present invention.

Pharmaceutical compositions may also contain diluents such as buffers, antioxidants such as ascorbic acid, low molecular weight (less than about 10 residues) polypeptides, proteins, amino acids, carbohydrates (e.g., glucose, sucrose, dextrins), chelating agents (e.g., EDTA), glutathione and other stabilizers and excipients. Neutral buffered saline or saline mixed with nonspecific serum albumin are exemplary diluents. Preferably, the product is formulated as a lyophilizate using appropriate excipient solutions (e.g., sucrose) as diluents.

Also contemplated is the administration of fusion protein compositions of this disclosure in combination with a second agent. A second agent may be one accepted in the art as a standard treatment for a particular disease state or disorder, such as in transplants, inflammation, and autoimmunity. Exemplary second agents contemplated include steroids, NSAIDs, mTOR inhibitors (e.g., rapamycin (sirolimus), temsirolimus, deforolimus, everolimus, zotarolimus, curcumin, farnesylthiosalicylic acid), calcineurin inhibitors (e.g., cyclosporine, tacrolimus), anti-metabolites (e.g., mycophenolic acid, mycophenolate mofetil), polyclonal antibodies (e.g., anti-thymocyte globulin), monoclonal antibodies (e.g., daclizumab, basiliximab), or other active and ancillary agents, or any combination thereof.

“Pharmaceutically acceptable salt” refers to a salt of a fusion protein, SMIP, or antibody of this disclosure that is pharmaceutically acceptable and that possesses the desired pharmacological activity of the parent compound. Such salts include the following: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, lauryl sulfuric acid, 3-phenylpropionic acid, trimethylacetic acid, tertiary butylacetic acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, or the like.

In particular illustrative embodiments, a fusion protein of this disclosure is administered intravenously by, for example, bolus injection or infusion. Routes of administration in addition to intravenous include oral, topical, parenteral (e.g., sublingually or buccally), sublingual, rectal, vaginal, and intranasal. The term parenteral as used herein includes subcutaneous injections, intravenous, intramuscular, intrasternal, intracavernous, intrathecal, intrameatal, intraurethral injection or infusion techniques. The pharmaceutical composition is formulated so as to allow the active ingredients contained therein to be bioavailable upon administration of the composition to a patient. Compositions administered to a patient can take the form of one or more dosage units, where, for example, a tablet may be a single dosage unit, or a container of one or more compounds of this disclosure in aerosol form may hold a plurality of dosage units.

For oral administration, an excipient and/or binder may be present, such as sucrose, kaolin, glycerin, starch dextran, cyclodextrin, sodium alginate, carboxy methylcellulose, and ethyl cellulose. Sweetening agents, preservatives, dye/colorant, flavor enhancer, or any combination thereof may optionally be present. A coating shell may also optionally be employed

In a composition intended to be administered by injection, one or more of a surfactant, preservative, wetting agent, dispersing agent, suspending agent, buffer, stabilizer, isotonic agent, or any combination thereof may optionally be included.

For nucleic acid-based formulations, or for formulations comprising expression products according to this disclosure, about 0.01 μg/kg to about 100 mg/kg body weight will be administered, for example, by the intradermal, subcutaneous, intramuscular, or intravenous route, or by any route known in the art to be suitable under a given set of circumstances. A preferred dosage, for example, is about 1 μg/kg to about 20 mg/kg, with about 5 μg/kg to about 10 mg/kg particularly preferred. It will be evident to those skilled in the art that the number and frequency of administration will be dependent upon the response of the host.

The pharmaceutical compositions of this disclosure may be in any form that allows for administration to a patient, such as, for example, in the form of a solid, liquid, or gas (aerosol). The composition may be in the form of a liquid, e.g., an elixir, syrup, solution, emulsion or suspension. The liquid may be for oral administration or for delivery by injection, as two examples.

A liquid pharmaceutical composition as used herein, whether in the form of a solution, suspension or other like form, may include one or more of the following components: sterile diluents such as water for injection, saline solution, preferably physiological saline, Ringer's solution, isotonic sodium chloride, fixed oils such as synthetic mono or digylcerides that may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium, chloride, or dextrose. The parenteral preparation can be enclosed in ampoules, disposable syringes or multiple dose vials made of glass or plastic. Physiological saline is a preferred additive. An injectable pharmaceutical composition is preferably sterile.

It may also be desirable to include other components in the preparation, such as delivery vehicles including aluminum salts, water-in-oil emulsions, biodegradable oil vehicles, oil-in-water emulsions, biodegradable microcapsules, and liposomes. Examples of adjuvants for use in such vehicles include N-acetylmuramyl-L-alanine-D-isoglutamine (MDP), lipopolysaccharides (LPS), glucan, IL-12, GM-CSF, γ-interferon, and IL-15.

While any suitable carrier known to those of ordinary skill in the art may be employed in the pharmaceutical compositions of this disclosure, the type of carrier will vary depending on the mode of administration and whether a sustained release is desired. For parenteral administration, the carrier may comprise water, saline, alcohol, a fat, a wax, a buffer, or any combination thereof. For oral administration, any of the above carriers or a solid carrier, such as mannitol, lactose, starch, magnesium stearate, sodium saccharine, talcum, cellulose, glucose, sucrose, magnesium carbonate, or any combination thereof, may be employed.

This disclosure contemplates a dosage unit comprising a pharmaceutical composition of this disclosure. Such dosage units include, for example, a single-dose or a multi-dose vial or syringe, including a two-compartment vial or syringe, one comprising the pharmaceutical composition of this disclosure in lyophilized form and the other a diluent for reconstitution. A multi-dose dosage unit can also be, e.g., a bag or tube for connection to an intravenous infusion device.

This disclosure also contemplates a kit comprising a pharmaceutical composition of this disclosure in unit dose, or multi-dose, container, e.g., a vial, and a set of instructions for administering the composition to patients suffering a disorder such as a disorder described above.

### EXAMPLES

### Monoclonal Antibodies and Exemplary Single Chain Fusion Proteins

Exemplary monoclonal antibodies (binding domains from which, and variants thereof, were used to make exemplary single chain fusion proteins) and single chain fusion proteins are briefly described herein.

Cris-7 (also referred to as Cris-7 mAb or Cris-7 FL) is a mouse anti-human CD3ε IgG2a monoclonal antibody (mAb) (Reinherz, E. L. et al. (eds.), Leukocyte typing II., Springer Verlag, New York, (1986)). The Cris-7 mAb was shown to bind to human, baboon, cynomolgous, and rhesus T cells (data not shown). Each of the Cris-7 single chain fusion proteins described herein was also shown to have this cross-species reactivity (data not shown).

Chimeric and humanized Cris-7 IgG1-N297A (SEQ ID NOS:265, 270, 275, 280, 285, 290, 295) comprise from amino-terminus to carboxyl-terminus: a chimeric or humanized Cris-7 heavy chain variable region, a linker that comprises three (Gly)₄-Ser linked in tandem, chimeric or humanized Cris-7 light chain variable region, a mutated IgG1 hinge region (SCC—P), the C_{H2} region of human IgG1 with an alanine substitution at position 297, and the C_{H3} region of human IgG1.

Chimeric and humanized Cris-7 IgG1-AA-N297A (SEQ ID NOS:266, 271, 276, 281, 286, 291, 296) comprise from amino-terminus to carboxyl-terminus: a chimeric or humanized Cris-7 heavy chain variable region, a linker that comprises three (Gly)₄-Ser linked in tandem, chimeric or humanized Cris-7 light chain variable region, a mutated IgG1 hinge region (SCC—P), the C_{H2} region of human IgG1 with four alanine substitutions at positions L234, L235, G237 and N297 and a deletion at G236 (i.e., LLGG(234-237)AAA), and the C_{H3} region of human IgG1.

Chimeric and humanized Cris-7 IgG2-AA-N297A (SEQ ID NOS:267, 272, 277, 282, 287, 292, 297) comprise from amino-terminus to carboxyl-terminus: a chimeric or humanized Cris-7 heavy chain variable region, a linker that comprises three (Gly)₄-Ser linked in tandem, chimeric or humanized Cris-7 light chain variable region, a mutated IgG1 hinge region (SCC—P), the C_{H2} region of human IgG2 with three alanine substitutions at positions V234, G236 and N297, and the C_{H3} region of human IgG2.

Chimeric and humanized Cris-7 IgG4-AA-N297A (SEQ ID NOS:268, 273, 278, 283, 288, 293, 298) comprise from amino-terminus to carboxyl-terminus: a chimeric or humanized Cris-7 heavy chain variable region, a linker that comprises three (Gly)₄-Ser linked in tandem, chimeric or humanized Cris-7 light chain variable region, a mutated IgG1 hinge region (SCC—P), the C_{H2} region of human IgG4 with four alanine substitutions at positions F234, L235, G237 and N297 and a deletion at G236 (i.e., FLGG(234-237)AAA), and the C_{H3} region of human IgG4.

Chimeric and humanized Cris-7 HM1 (SEQ ID NOS:269, 274, 279, 284, 289, 294, 299) comprise from amino-terminus to carboxyl-terminus: a chimeric or humanized Cris-7 heavy chain variable region, a linker that comprises at least three (Gly)₄-Ser linked in tandem, Cris-7 light chain variable region, wild type human IgG1 hinge region, the C_{H3} region from human IgM, and the C_{H3} region from human IgG1, and a tail sequence that comprises three copies of the FLAG epitope, one copy of the AVI tag, and six histidines.

BC3 (also referred to as BC3 mAb or BC3 FL) is a non-mitogenic mouse anti-human CD3ε IgG2b mAb (Anasetti et al., J. Exp. Med. 172: 1691-1700, 1990).

BC3-HM1 (also referred to as “BC3 HM1”) (SEQ ID NO:84) comprises from its amino-terminus to carboxyl-terminus: BC3 heavy chain variable region, a linker that comprises at least three (Gly)₄-Ser linked in tandem, BC3 light chain variable region, wild type human IgG1 hinge region, the C_{H3} region from human IgM, and the C_{H3} region from human IgG1, and a tail sequence that comprises three copies of the FLAG epitope, one copy of the AVI tag, and six histidines.

BC3-ΔC_{H2} (also referred to as “BC3 ΔC_{H2}”) (SEQ ID NO:85) comprises from its amino-terminus to carboxyl-terminus: BC3 heavy chain variable region, a linker that comprises at least three (Gly)₄-Ser linked in tandem, BC3 light chain variable region, wild type IgG1 hinge region, the C_{H3} region of human IgG1, and a tail sequence that comprises three copies of the FLAG epitope, one copy of the AVI tag, and six histidines.

BC3-G1 N297A (also referred to as “BC3 N297A”) (SEQ ID NO:80) comprises from its amino-terminus to carboxyl-terminus: BC3 heavy chain variable region, a linker that comprises three (Gly)₄-Ser linked in tandem, BC3 light chain variable region, a mutated IgG1 hinge region (SCC—P), the C_{H2} region of human IgG1 with an alanine substitution at the asparagine of position 297, and the C_{H3} region of human IgG1.

BC3-G1 AA N297A (also referred to as “BC3 IgG1AA”) (SEQ ID NO:81) comprises from its amino terminus to carboxyl terminus: BC3 heavy chain variable region, a linker that comprises three (Gly)₄-Ser linked in tandem, BC3 light chain variable region, a mutated IgG1 hinge region (SCC—P), the C_{H2} region of human IgG1 with four alanine substitutions at positions L234, L235, 237 and N297 and a deletion at G236 (i.e., LLGG(234-237)AAA), and the C_{H3} region of human IgG1.

BC3-G2 AA N297A (also referred to as “BC3 IgG2AA”) (SEQ ID NO:82) comprises from its amino terminus to carboxyl terminus: BC3 heavy chain variable region, a linker that comprises three (Gly)₄-Ser linked in tandem, BC3 light chain variable region, a mutated IgG1 hinge region (SCC—P), the C_{H2} region of human IgG2 with three alanine substitutions at positions V234, G236 and N297, and the C_{H3} region of human IgG2.

BC3-G4 AA N297A (also referred to as “BC3 IgG4AA”) (SEQ ID NO:83) comprises from its amino terminus to carboxyl terminus: BC3 heavy chain variable region, a linker that comprises three (Gly)₄-Ser linked in tandem, BC3 light chain variable region, a mutated IgG1 hinge region (SCC—P), the C_{H2} region of human IgG4 with four alanine substitutions at positions F234, L235, G237 and N297 and a deletion at G236 (i.e., FLGG(234-237)AAA), and the C_{H3} region of human IgG4.

OKT3 (also referred to as OKT3 mAb or OKT3 FL) is a mitogenic mouse anti-human CD3ε IgG2a mAb (Ortho Multicencer Transplant Study Group, N. Engl. J. Med. 313: 337, 1985).

OKT3-HM1 (also referred to as “OKT3 HM1”) (SEQ ID NO:92) comprises from its amino-terminus to carboxyl-terminus: OKT3 heavy chain variable region, a linker that comprises at least three (Gly)₄-Ser linked in tandem, OKT3 light chain variable region, wild type human IgG1 hinge region, the C_{H3} region from human IgM, and the C_{H3} region from human IgG1, and a tail sequence that comprises three copies of the FLAG epitope, one copy of the AVI tag, and six histidines.

OKT3-ΔC_{H2} (also referred to as “OKT ΔC_{H2}”) (SEQ ID NO:93) comprises from its amino-terminus to carboxyl-terminus: OKT3 heavy chain variable region, a linker that comprises at least three (Gly)₄-Ser linked in tandem, OKT3 light chain variable region, wild type IgG1 hinge region, the C_{H3} region of human IgG1, and an additional tail sequence that comprises three copies of the FLAG epitope, one copy of the AVI tag, and six histidines. OKT3-G1 N297A (also referred to as “OKT N297A”) (SEQ ID NO:88) comprises from its amino-terminus to carboxyl-terminus: OKT3 heavy chain variable region, a linker that comprises three (Gly)₄-Ser linked in tandem, OKT3 light chain variable region, a mutated IgG1 hinge region (SCC—P), the C_{H2} region of human IgG1 with an alanine substitution at position 297, and the C_{H3} region of human IgG1.

OKT3-G1 AA N297A (also referred to as “OKT3 IgG1AA”) (SEQ ID NO:89) comprises from its amino terminus to carboxyl terminus: a leader sequence derived from human 2H7 leader sequence, OKT3 heavy chain variable region, a linker that comprises three (Gly)₄-Ser linked in tandem, OKT3 light chain variable region, a mutated IgG1 hinge region (SCC—P), the C_{H2} region of human IgG1 with four alanine substitutions at positions L234, L235, G237 and N297 and a deletion at G236 (i.e., LLGG(234-237)AAA), and the C_{H3} region of human IgG1.

OKT3-G2 AA N297A (also referred to as “OKT3 IgG2AA”) (SEQ ID NO:90) comprises from its amino terminus to carboxyl terminus: OKT3 heavy chain variable region, a linker that comprises three (Gly)₄-Ser linked in tandem, OKT3 light chain variable region, a mutated IgG1 hinge region (SCC—P), the C_{H2} region of human IgG2 with three alanine substitutions at positions V234, G236 and N297, and the C_{H3} region of human IgG2.

OKT3-G4 AA N297A (also referred to as “OKT3 IgG4AA”) (SEQ ID NO:91) comprises from its amino terminus to carboxyl terminus: OKT3 heavy chain variable region, a linker that comprises three (Gly)₄-Ser linked in tandem, OKT3 light chain variable region, a mutated IgG1 hinge region (SCC—P), the C_{H2} region of human IgG4 with four alanine substitutions at positions F234, L235, G237 and N297 and a deletion at G236 (i.e., FLGG(234-237)AAA), and the C_{H3} region of human IgG4.

Also made and tested were OKT3 IgG4-N297A (i.e., the C_{H2} region of human IgG4 having only the N297A substitution, also known as OKT3 IgG4-WT-N297A or OKT3 IgG4-FLGG-N297A; SEQ ID NO:232, which sequence includes a 22 amino acid leader sequence that is not a part of the mature fusion protein). Also, single alanine substitution mutations at each of the four positions (F234, L235, G236 and G237) in combination with the N297A substitution were made (i.e., OKT3 IgG4-ALGG-N297A, OKT3 IgG4-FAGG-N297A, OKT3 IgG4-FLAG-N297A, and OKT3 IgG4-FLGA-N297A, which correspond to SEQ ID NOS:234, 236, 238, and 240, respectively—these also include a 22 amino acid leader sequence that is not a part of the mature fusion protein).

OKT3 ala-ala (also referred to as OKT3 AA-FL or OKT3 FL) is a humanized, Fc mutated anti-CD3 mAb that contains alanine substitutions at positions 234 and 235 (Herold et al. (2003) J. Clin. Invest. 11(3): 409-18).

Visilizumab (also referred to as “Nuvion FL”) is a humanized, Fc mutated anti-CD3 mAb directed against the CD3ε chain of the TCR. It is a human IgG2 isotype and contains mutations at positions 234 and 237 (Carpenter et al., Blood 99: 2712-9, 2002).

H57-457 mAb is a hamster anti-TCR monoclonal antibody. It is mitogenic and functions similarly to OKT3 monoclonal antibody (Lavasani et al. (2007) Scandinavian Journal of Immunology 65:39). The sequences of V_{H} and V_{L} regions of H57-457 mAb are set forth in SEQ ID NOS:49 and 51.

H57 half null (SEQ ID NO:304) is a mouse IgG2a single chain fusion protein having H57 binding domain and with mutations in C_{H2} that cause the loss of ADCC activities in addition to the N297A substitution. It comprises from its amino terminus to carboxy terminus: H57 heavy chain variable region, a linker that comprises three (Gly)₄-Ser linked in tandem, H57 light chain variable region, a wild type mouse IGHG2c hinge region, the C_{H2} region of mouse IGHG2c with four alanine substitutions at positions L234, L235, G237, and N297, and the C_{H3} region of mouse IGHG2c.

H57 HM2 (SEQ ID NO:306) is a mouse single chain fusion protein that comprises from its amino terminus to carboxy terminus: H57 heavy chain variable region, a linker that comprises three (Gly)₄-Ser linked in tandem, H57 light chain variable region, a wild type mouse IGHG2c hinge region, the mouse C_{H3μ} region, and the mouse C_{H37} region.

H57 Null2 (SEQ ID NO:96) is a mouse IgG2a single chain fusion protein having H57 binding domain and with mutations in C_{H2} that cause the loss of ADCC and CDC activities. It comprises from its amino terminus to carboxy terminus: H57 heavy chain variable region, a linker that comprises three (Gly)₄-Ser linked in tandem, H57 light chain variable region, a wild type mouse IGHG2c hinge region, the C_{H2} region of mouse IGHG2c with six alanine substitutions at positions L234, L235, G237, E318, K320, and K322, and the C_{H3} region of mouse IGHG2c.

145-2C11 mAb (also referred to as 2C11 mAb) is a hamster monoclonal antibody against the CD3ε chain of the murine TCR complex (Hirsch et al., J. Immunol. 140: 3766, 1988). It is also mitogenic and functions similar to OKT3 monoclonal antibody. The sequences of V_{H} and V_{L} regions of 145-2C11 mAb are set forth in SEQ ID NOS:58 and 60.

2C11 Null2 (SEQ ID NO:56) is a mouse IgG2a single chain fusion protein having 2C11 binding domain and with mutations in C_{H2} which cause the loss of ADCC and CDC activities. It comprises from its amino terminus to carboxy terminus: 2C11 heavy chain variable region, a linker that comprises three (Gly)₄-Ser linked in tandem, 2C11 light chain variable region, a wild type mouse IGHG2c hinge region, the C_{H2} region of mouse IGHG2c with six alanine substitutions at positions L234, L235, G237, E318, K₃₂₀, and K₃₂₂, and the C_{H3} region of mouse IGHG2c.

### Example 1

### Fusion Proteins do not Activate Primed T cells or induce cytokine release By Primed T Cells or Accessory Cells

### Isolation of Human Peripheral Blood Mononuclear Cells (PBMC)

Fresh human whole blood was obtained in 30 mL syringes containing heparin (up to 25 mL blood per syringe) and was kept at room temperature up 2 hours before processing. The blood was diluted in a 50 mL conical tube with an equal volume of room temperature RPMI-1640 (no supplements). The diluted blood was mixed 2 to 3 times by gentle inversion. Using a 25 mL pipette, 20 to 25 mL of the diluted blood was layered carefully over 15 mL of Lymphocyte Separation Media (MP Biomedicals) contained in a 50 mL conical tube. The tubes were centrifuged at 400 g for 30 minutes at room temperature. Cells were collected from the interface of the density gradient and were combined in a 50 mL conical tube, with no more than 30 mL of cell suspension per tube. The tubes containing the cell suspensions were filled with RPMI-1640 containing 10% FBS, 100 U/mL penicillin, 100 ug/mL Streptomycin, and 2 mM L-glutamine (Complete RPMI-1640). The tubes were centrifuged at 1500 rpm for 5 minutes at room temperature and the supernatant was aspirated. The cells were washed twice by resuspending them in 20 mL of Complete RPMI, centrifuging at 1500 rpm for 5 minutes at room temperature, and aspirating the supernatant. The washed cells were counted by hemacytometer and resuspended according the assay protocol for which they were being used.

Labeling Human PBMC with Carboxyfluorescein Succinimidyl Ester (CFSE)

The density of mouse splenocytes was adjusted to 1×10⁶/mL in sterile PBS. The cells were distributed into 50 mL conical tubes with no more than 25 mL (25×10⁶ cells) per tube. The cells were labeled with CFSE using the CELLTRACE™ CFSE Cell Proliferation Kit (Molecular Probes), after optimizing conditions for use. A 5 mM solution of CFSE in tissue culture grade DMSO was prepared immediately before use by adding 18 uL of high grade DMSO (Component B of kit) to a vial containing 50 μg of lyophilized CFSE (Component A of kit). The CFSE solution was added to the PBMC cell suspensions to a final concentration of 50 nM CFSE, then the cell suspensions were incubated at 37° C. in 5% CO₂ for 15 minutes. The cell labeling reaction was quenched by filling the tubes with RPMI Complete (RPMI-1640 containing 10% FBS, 100 U/mL penicillin, 100 ug/mL Streptomycin, and 2 mM L-glutamine). The cells were spun at 1500 rpm for 7 minutes at room temperature. The supernatant was aspirated from each tube and the cells were re-suspended in RPMI Complete. The cells were counted and adjusted in RPMI Complete to the desired density for use in assays.

### Analysis of Mitogenicity and Cytokine Release Using PHA-Primed T Cells

Human PBMC were suspended at a concentration of 2×10⁶ cells/mL in complete RPMI media (RPMI-1640 containing 10% Human AB serum, 100 U/mL penicillin, 100 μg/mL Streptomycin, and 2 mM L-glutamine) and stimulated with 2.5 μg/mL of PHA (Sigma) at 37° C. for 3 days. After incubation, cells were washed twice with complete RPMI and re-plated at a concentration of about 2×10⁶ cells/mL in a new flask with no stimulation. Cells were then placed at 37° C. for an additional 4 days, allowing the T cells to rest before exposure to a secondary stimulus. At the end of this 4 day rest period, cells were harvested, washed with PBS, and labeled with CFSE as previously described. After labeling, cells were suspended at a concentration of 2×10⁶ cells/ml in complete (human serum) RPMI (RPMI-1640 containing 10% human AB serum, 100 U/mL penicillin, 100 ug/mL Streptomycin, and 2 mM L-glutamine). At this time, fresh PBMCs were isolated from the same donor and used as accessory cells for restimulation. To prepare the accessory cells, T cells were magnetically separated from the PBMC population using the EasySep technology (Stem Cell Technologies Cat#18051). Magnetic nanoparticles along with dextran and a cocktail of antibodies directed against CD3 were incubated with the freshly isolated PBMCs according to the manufacturer's protocol. The cell and bead mixture was then left in a first tube with EasySep Purple magnet for 5 minutes and then the cell suspension was poured into a second 5 mL FACS tube. The CD3′ cells (T cells) were retained in the first tube, while the accessory cells were transferred into the second tube. The negatively selected accessory cells were treated with mitomycin C (MMC, as described below) to inhibit proliferation. Both CFSE-labeled PHA blasts and MMC treated accessory cells were suspended in complete (human AB serum) RPMI at 2×10⁶ cells/mL. Each cell population was added to a 48-well tissue culture plate (0.5 mL/well) along with the indicated treatments. Cells were incubated for an additional 4 days at 37° C. and 50 μL of supernatant was harvested at 24 hrs after stimulation. The cells and remaining supernatant were harvested on Day 4 post-restimulation. Harvested cells were stained with fluorescently tagged antibodies against CD5 (340697, BDBiosciences) CD25 (557741, BDBiosciences) and 7AAD (559925, BD Biosciences) and run through a flow cytometer (LSR11, Becton Dickenson). Data was analyzed using FlowJo flow cytometry software (TreeStar). The gating strategy was as follows: cells that fell within a forward scatter (FSC): side scatter (SSC) lymphocyte gate were analyzed for 7AAD expression. Cells that fell into the 7AAD negative gate were then analyzed for CD5 expression, and cells that were within the CD5+ gate were then analyzed for CFSE dilution and CD25 upreguation. Cells that were CD5+, CFSE^{lo} and CD25^{hi} were considered activated T cells. Supernatant samples were analyzed for the presence of cytokines and chemokines using a custom 11-plex Luminex-based detection kit from Millipore (Milliplex series), following the manufacturer's procotol. The 11 analytes detected by the kit were: IL-β, IL-1RA, IL-2, IL-4, IL-6, IL-10, IL-17, IP-10, MCP1, IFNγ, and TNFα.

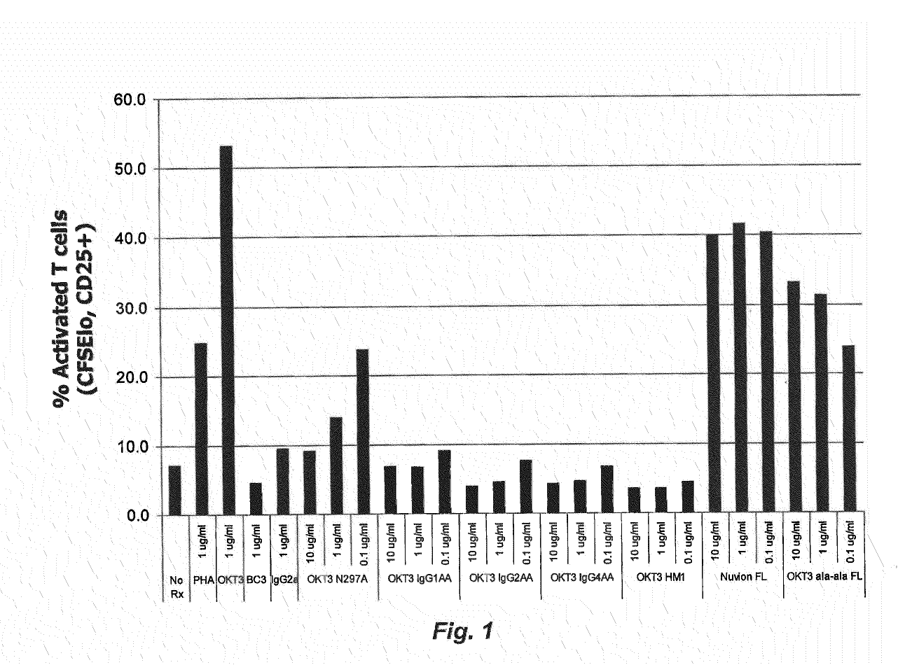

FIG. 1 shows that the OKT3 IgG2AA, OKT3 IgG4AA, and OKT3 HM1 fusion proteins did not activate PHA-primed T cells as compared to known antibodies visilizumab and OKT3 ala-ala. Similar data were generated with molecules containing the BC3 binding domain.

Table 1 shows that OKT3 IgG2AA, OKT3 IgG4AA and OKT3 HM1 fusion proteins did not induce cytokine release by primed T cells or accessory cells, in contrast to known antibodies visilizumab and OKT3 ala-ala.

TABLE 1
Cytokine Data

| | | | IL-1b | IL-2 | IL-4 | IL-6 | IL-10 | IL-17 | IFN-g | TNF-a | MCP-1 | IP-10 | IL-1RA |
|---|---|---|---|---|---|---|---|---|---|---|---|---|---|
| untreated | | | 20.0 | 2.1 | 7.4 | 1580.4 | 469.6 | 27.1 | 13.6 | 195.2 | 1393.9 | 234.1 | 4227.5 |
| PHA | 2.5 | ug/mL | 22.9 | 7.7 | 53.6 | 2293.6 | 1498.1 | 59.4 | 41.9 | 159.7 | 1467.2 | 625.0 | 5760.6 |
| OKT3 mAb | 10 | ug/mL | 77.6 | 101.0 | 168.0 | 3300.2 | 8196.0 | 203.0 | 971.6 | 877.9 | 2310.7 | 1010.7 | 9097.7 |
| BC3 mAb | 10 | ug/mL | 14.7 | 0.2 | 1.1 | 1440.0 | 332.5 | 3.2 | 1.8 | 222.7 | 1899.4 | 73.9 | 4330.3 |
| IgG2a | 10 | ug/mL | 46.1 | 3.5 | 6.4 | 3926.0 | 980.7 | 36.7 | 31.4 | 281.9 | 1566.5 | 368.8 | 5503.0 |
| OKT3 N297A | 7.3 | ug/ml | 44.5 | 1.5 | 6.2 | 1677.8 | 401.8 | 17.8 | 17.7 | 341.9 | 1702.5 | 272.9 | 8803.2 |
| | .73 | ug/ml | 31.5 | 3.2 | 14.6 | 1625.5 | 649.9 | 35.7 | 35.9 | 365.0 | 1508.5 | 433.3 | 8523.6 |
| | .073 | ug/ml | 26.8 | 6.5 | 31.7 | 1784.8 | 1642.0 | 67.2 | 74.8 | 358.3 | 1637.3 | 775.6 | 8072.2 |
| OKT3 IgG1AA | 7.3 | ug/ml | 474.4 | 0.8 | 5.0 | 21297.4 | 9133.1 | 6.6 | 142.5 | 2082.9 | 2973.3 | 111.4 | 10077.3 |
| | .73 | ug/ml | 109.8 | 0.3 | 3.7 | 14723.7 | 2088.2 | 5.8 | 17.3 | 375.2 | 1777.4 | 145.5 | 5081.0 |
| | .073 | ug/ml | 24.6 | 0.6 | 3.9 | 1805.7 | 454.6 | 13.0 | 13.2 | 180.7 | 1401.7 | 352.3 | 4188.9 |
| OKT3 IgG2AA | 7.3 | ug/ml | 19.8 | 0.4 | 4.1 | 1345.3 | 280.2 | 4.0 | 1.5 | 144.8 | 1675.2 | 106.6 | 3884.5 |
| | .73 | ug/ml | 19.6 | 0.4 | 3.2 | 1701.8 | 278.9 | 5.1 | 3.2 | 126.9 | 1518.3 | 143.7 | 3152.2 |
| | .073 | ug/ml | 17.9 | 0.7 | 2.8 | 1659.4 | 305.3 | 11.4 | 6.9 | 160.7 | 1517.7 | 282.3 | 3586.4 |
| OKT3 IgG4AA | 7.3 | ug/ml | 20.3 | 0.3 | 2.6 | 1632.4 | 265.0 | 4.1 | 2.1 | 140.4 | 1081.2 | 76.8 | 3020.9 |
| | .73 | ug/ml | 17.6 | 0.4 | 0.5 | 1532.5 | 249.8 | 6.2 | 4.9 | 155.3 | 1281.8 | 231.8 | 3639.6 |
| | .073 | ug/ml | 24.5 | 0.4 | 0.0 | 1470.7 | 294.7 | 9.2 | 5.9 | 163.7 | 1307.5 | 167.1 | 3346.4 |
| OKT3 HM1 | 7.3 | ug/ml | 9.2 | 0.2 | 3.2 | 862.1 | 185.6 | 1.2 | 0.8 | 122.4 | 1128.9 | 34.8 | 3118.8 |
| | .73 | ug/ml | 13.7 | 0.2 | 1.1 | 1045.2 | 233.8 | 1.6 | 0.8 | 131.1 | 986.7 | 40.2 | 3284.7 |
| | .073 | ug/ml | 17.3 | 0.6 | 0.0 | 1743.4 | 274.3 | 8.2 | 2.4 | 149.1 | 1216.2 | 107.4 | 3260.2 |
| Nuvion FL | 10 | ug/ml | 12.8 | 7.9 | 63.0 | 2149.0 | 2132.3 | 65.9 | 92.6 | 245.9 | 1732.0 | 972.5 | 7923.9 |
| | 1 | ug/ml | 18.7 | 10.0 | 57.1 | 1936.9 | 2129.4 | 78.1 | 100.6 | 245.8 | 1791.8 | 1207.6 | 6553.7 |
| | .1 | ug/ml | 19.8 | 7.6 | 43.8 | 2204.3 | 2076.4 | 73.5 | 99.0 | 274.9 | 1273.0 | 1386.4 | 7469.3 |
| OKT3 ala-ala FL | 10 | ug/ml | 38.2 | 6.9 | 44.4 | 2033.5 | 2052.8 | 82.1 | 105.6 | 373.7 | 2309.6 | 720.8 | 7791.7 |
| | 1 | ug/ml | 32.3 | 6.8 | 43.2 | 2958.7 | 1999.5 | 82.7 | 102.6 | 392.7 | 2812.7 | 841.2 | 8950.2 |
| | .1 | ug/ml | 28.0 | 7.3 | 32.0 | 2710.9 | 1595.1 | 74.0 | 66.4 | 268.7 | 2692.8 | 631.8 | 6825.3 |

### Example 2

### Fusion Proteins Block a T Cell Response to Alloantigen

### Human Mixed Lymphocyte Reaction (MLR)

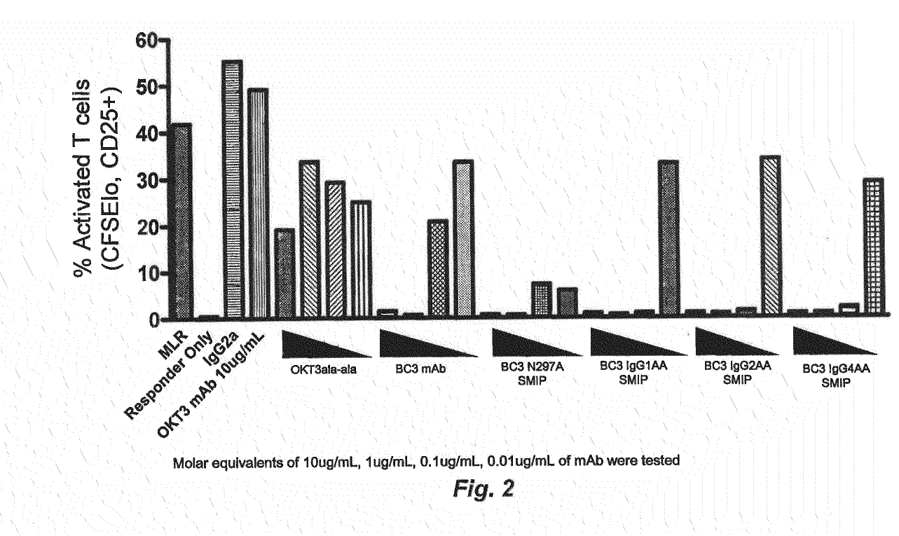

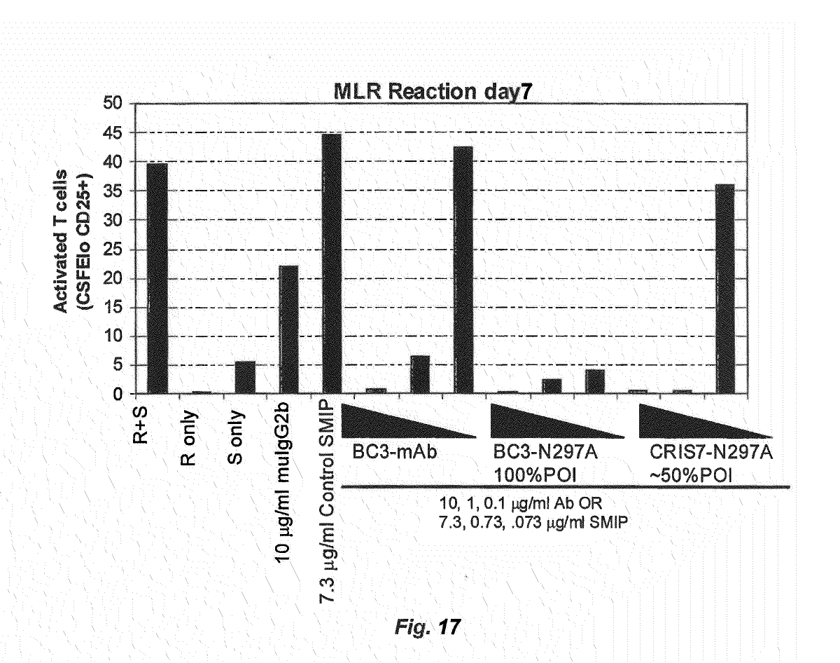

Human PBMCs from two donors were isolated as described previously and kept separate. Based on previous studies, PBMCs from one donor were slated to be the stimulator population and PBMCs for the second donor were used as the responder population. Cells from both donors were labeled with CFSE as previously described. The PBMCs from the donor to be used as the stimulator were treated with mitomycin C (MMC) to prevent cell division. MMC (Sigma) was resuspended in complete (HS) RPMI media (RPMI-1640 containing 10% human AB serum, 100 U/mL penicillin, 100 ug/mL Streptomycin, and 2 mM L-glutamine) at a concentration of 0.5 mg/mL. PBMCs were resuspended at a concentration of about 1×10⁶/mL and MMC was added to a final concentration of 25 μg/mL. The cell and MMC mixture was then incubated at 37° C. for 30 minutes after which time cells were washed thrice with complete (HS) RPMI media. Prepared stimulator and responder cells were suspended at a concentration of about 2×10⁶/mL in complete (human AB serum) RPMI (RPMI-1640 containing 10% human AB serum, 100 U/mL penicillin, 100 μg/mL Streptomycin, and 2 mM L-glutamine) and 0.25 mL of each cell population was added per well of a 48-well plate. All treatments were added to the plate at the same time as the cells (at the concentrations shown in FIGS. 2, 3, and 17; note that the concentrations given are for antibodies and that molar equivalent concentrations were used for the fusion proteins as shown in FIG. 17) and samples were then incubated at 37° C. for the duration of the experiment. Experiments were harvested 7-8 days after set-up. Harvested cells were stained with fluorescently tagged antibodies against CD5 (340697, BDBiosciences), CD25 (555433, BDBiosciences), and 7AAD (559925, BD Biosciences), and run on a flow cytometer (LSR11, Becton Dickenson). Data was analyzed using FlowJo flow cytometry software (TreeStar). The gating strategy was as follows: cells that fell within a FSC:SSC lymphocyte gate were analyzed for 7AAD expression. Cells that fell within the negative 7AAD gate were then analyzed for CD5+ expression, and cells that were CD5+ were then analyzed for CFSE dilution and CD25 up-regulation. Cells that were CD5+, CFSE^{lo} and CD25^{hi} were considered activated T cells.

FIG. 2 shows that the BC3 IgG2AA and BC3 IgG4AA fusion proteins blocked a T cell response to alloantigen better than known BC3 mAB and in contrast to OKT3 ala-ala antibody. Similar data were generated with molecules expressing the OKT3 binding domain.

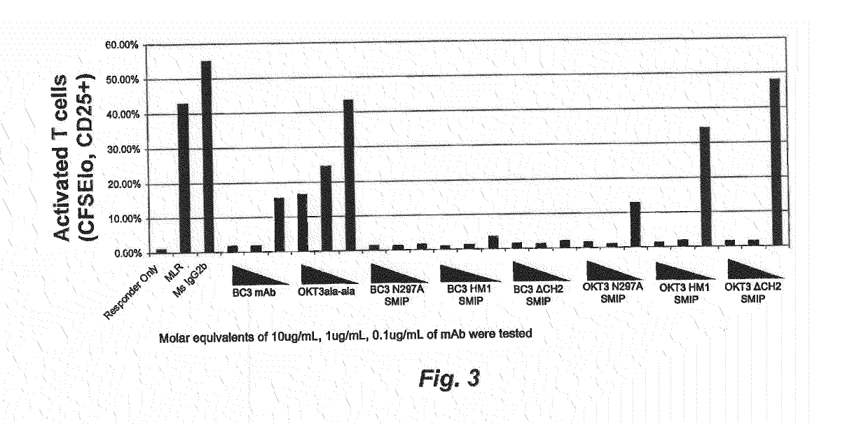

FIG. 3 shows that the BC3 HM1 and BC3 AC_{H2} fusion proteins also blocked a T cell response to alloantigen. Similar data were generated with molecules expressing the OKT3 binding domain.

FIG. 17 shows that a partially purified Cris-7 IgG1-N297A (50% is the peak of interest) effectively blocked a T cell response to alloantigen.

### Example 3

### Fusion Proteins Block Memory T Cell Response to Recall Antigen

Human PBMCs were isolated from a donor that scored positive in a previous screen for reactivity to tetanus toxoid. PBMCs were labeled with CFSE as previously described and then resuspended at a concentration of 2×10⁶/mL in complete (human AB serum) RPMI (RPMI-1640 containing 10% human AB serum, 100 U/mL penicillin, 100 μg/mL Streptomycin, and 2 mM L-glutamine). 0.5 mL of CFSE-labeled cells and 1 ug/mL of tetanus toxoid (EMD), along with experimental treatments, were added to a 48-well plate. The cells were incubated at 37° C. with 5% CO₂ for the duration of the experiment. Experiments were harvested 8 days after set-up. Harvested cells were stained with fluorescently tagged antibodies against CD5 (340697, BDBiosciences) and CD25 (555433, BDBiosciences) and run on a flow cytometer (LSR11, Becton Dickenson). Data was analyzed using FlowJo flow cytometry software (TreeStar). The gating strategy was as follows: cells that fell within a FSC:SSC lymphocyte gate were analyzed for CD5 expression, cells that subsequently fell within the CD5+ gate were then analyzed for CFSE dilution and CD25 upregulation. Cells that were CD5+, CFSE^{lo} and CD25^{hi} were considered activated T cells.

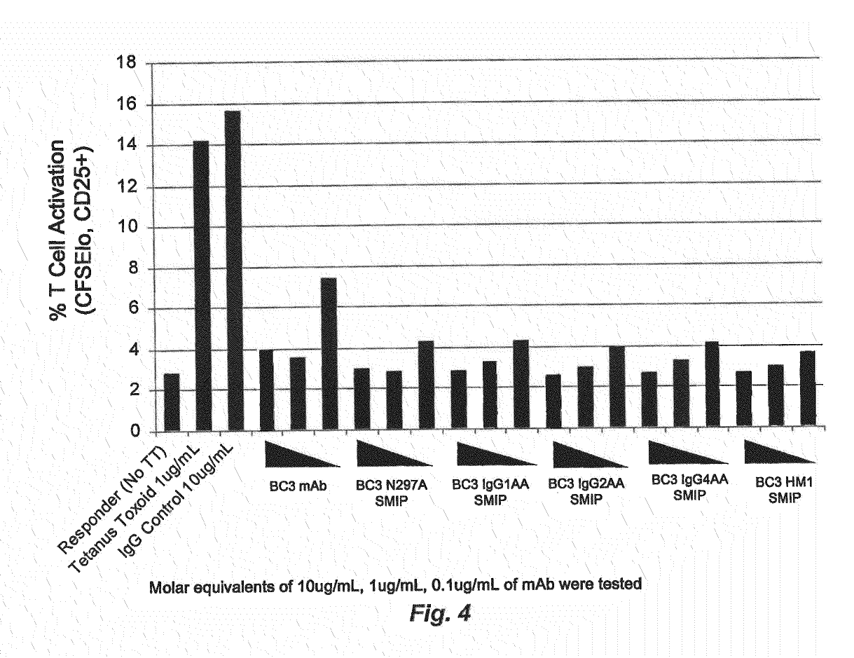

FIG. 4 shows that the BC3 IgG2AA, BC3 IgG4 AA, and BC3 HM1 fusion proteins can block a memory T cell response to a recall antigen, tetanus toxoid. Similar data were generated with fusion proteins containing the OKT3 binding domain.

### Example 4

### Fusion Proteins Induce Downmodulation of Cell Surface TCR and CD3

Human PBMCs were isolated as described in Example 1 and suspended at a concentration of about 2×10⁶ cells/mL. A portion of the PBMCs were set aside for immediate cell surface staining while the rest of the PBMCs were incubated with various anti-CD3 reagents for 4 days before analysis. PBMCs to be stained immediately were cooled on ice for 30 minutes after which they were spun down at 1500 rpm for 10 min at 4° C. and the resulting supernatant was removed. Cells were suspended in ice cold FACS Buffer (dPBS, 2.5% FBS) at a concentration of 1×10⁶/mL. 1 mL of cells was transferred into a 5 mL FACS tube (BD Falcon) for each reagent to be analyzed. An additional 1 mL of ice cold FACS Buffer was added to the 1 mL aliquots of cells and the cells were spun down at 1500 rpm for 5 minutes at 4° C. Tubes were inverted and supernatant decanted so that there was approximately 0.1 mL of FACS Buffer left in the tube along with the cell pellet and the tubes were then placed on ice. A master stock of staining antibodies (90 μL of ice cold FACS buffer, 5 μL anti-CD5 antibody (eBioscience), and 5 μL anti-TCR antibody (BDBiosciences)) was prepared to analyze samples immediately after isolation. Master stock (100 μL) was added to each FACS tube, along with 1 ug/mL, 0.5 μg/mL, or 0.1 μg/mL of the CD3-directed fusion proteins or monoclonal antibody (note that the concentrations given are for antibodies and that molar equivalent concentrations were used for the fusion proteins). The samples were then incubated on ice, in the dark for 30 minutes. After the incubation period, samples were washed twice with 2 mL ice cold FACS Buffer and a PE-labeled secondary antibody specific for the CD3-directed reagent was added at a final dilution of 1:400. The samples were then incubated on ice, in the dark for 30 minutes, and then washed twice with 2 mL ice cold FACS buffer. Staining levels were measured on an LSR11 flow cytometer (Becton Dickenson).

PBMCs to be treated for 4 days and then cell surface stained were plated in 0.5 mL aliquots per well (cell concentration was about 2×10⁶ cells/mL in complete (human AB serum) RPMI media) in 48-well plates. CD3-directed reagents were added to the cells at 1, 0.5 and 0.1 μg/mL (note that the concentrations given are for antibodies and that molar equivalent concentrations were used for fusion proteins) and the cells were incubated at 37° C. for 2 to 4 days. After incubation, cells were harvested and the stained as described above.

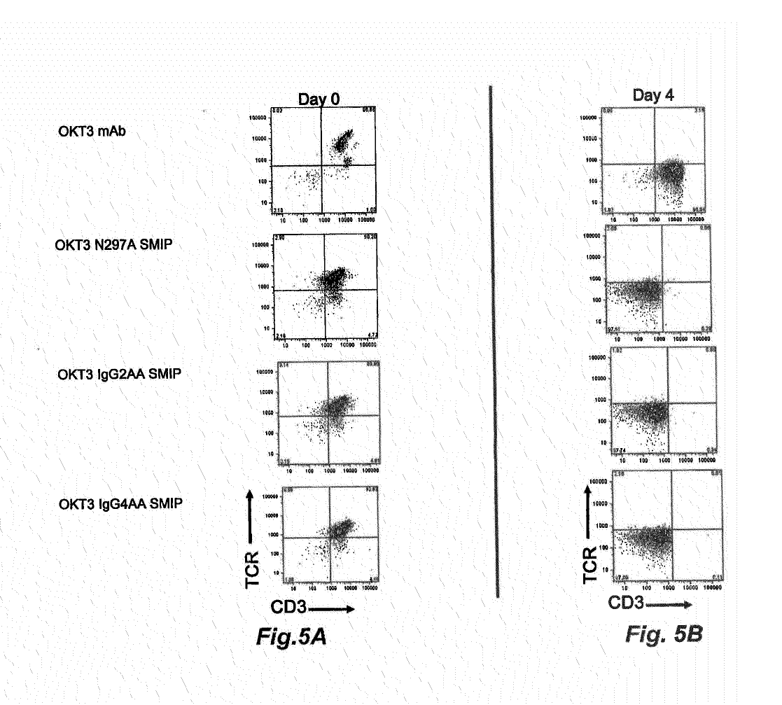

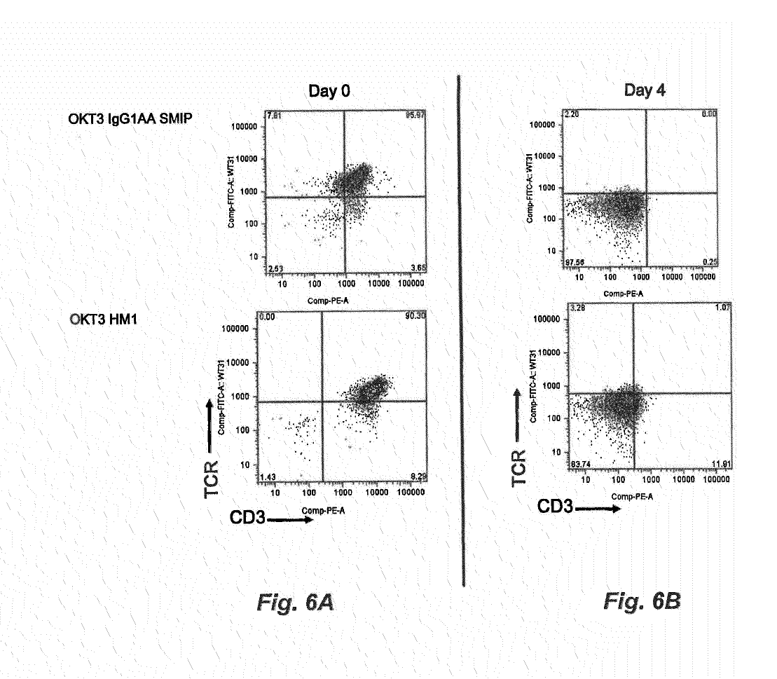

The results (FIGS. 5A, 5B, 6A and 6B) show that fusion proteins comprising the OKT3 binding domain induce the downmodulation of both the TCR and CD3 from the surface of T cells, while OKT3 monoclonal antibody only downmodulated the TCR and not CD3. Similar results were obtained with fusion proteins comprising the BC3 binding domain.

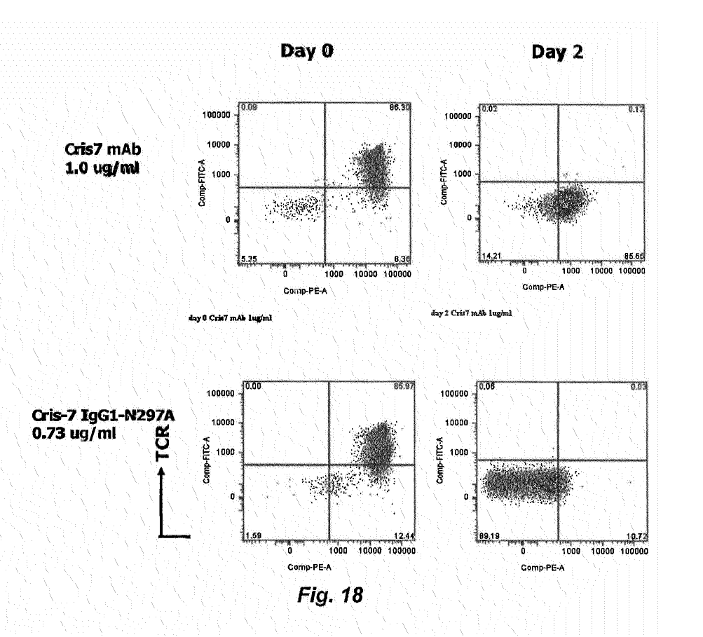

FIG. 18 shows that the Cris-7 IgG1-N297A fusion protein induces downmodulation of both the TCR and CD3 from the T cell surface, while the Cris-7 monoclonal antibody only downmodulates the TCR. Similar results are obtained with Cris-7 IgG2-AA-N297A, Cris-7 IgG4-AA-N297A, and Cris-7 HM1.

### Example 5

### Fusion Proteins Induce a Robust Calcium Flux in T Cells

Human PBMCs were isolated as previously described. Non-T cells were magnetically separated from T cells using the MACS technology from Miltenyi. Untouched T cells were isolated with The Pan T Cell Isolation Kit II (Miltenyi). Supermagnetic beads coated with a panel of antibodies directed against all cellular subsets of PBMCs except T cells were incubated with the freshly isolated PBMCs according to the manufacturer's protocol. The cell and bead mixture was then applied to a column containing a matrix that forms a magnetic field when placed in a MACS Separater (Miltenyi), a strong permanent magnet. The T cells flow through the column while all other cells are retained in the column. T cell purity was generally between 87-93%. The purified T cells were suspended in complete RPMI (RPMI-1640, 10% human AB serum, 2 mM L-glutamine, sodium pyruvate, non-essential amino acids, penicillin/streptomycin) at a concentration of about 2×10⁶ cells/mL and incubated at 37° C. in an appropriately sized flask overnight. The following morning, 100 μl of cells (200,000 cells) were transferred into the wells of a 96-well, black, poly-D lysine plate and incubated at 37° C. for 3 hours. During this incubation time, the calcium flux indicator dye was prepared according to manufacturer's instructions (Molecular Devices FLIPR Calcium 4 assay). In addition, experimental treatments were prepared in U-bottom plates. Cell treatments were prepared at a 5× concentration in the treatment plate in a 75 μL volume. All treatments (fusion proteins and cross-linkers) were tested in triplicate. 100 μL it of indicator dye was added to the cells one hour prior to reading the plate. After the addition of indicator dye, the plate was placed back in the incubator for an additional 45 minutes. Plates were then spun at 1200 rpm for 5 minutes at room temperature and then returned to the incubator for an additional 15 minutes. At the end of this incubation period, the treatment plate and cell plate were loaded into the FlexStation 3 (Molecular Devices), a benchtop plate reader with integrated fluid transfer. The Flexstation robotically added 50 uL of treatment to the cell plate and then recorded the resulting fluorescence from the calcium indicator dye every 7 seconds over the course of 750 sec. Captured data was then exported to Excel (Microsoft Office) for analysis.

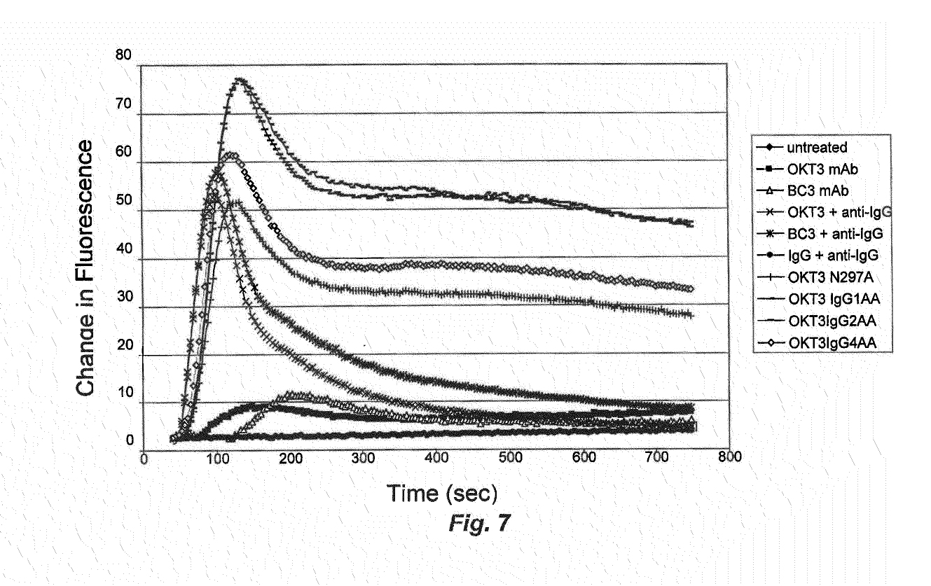

The results (FIG. 7) show that, in contrast to antibodies having the same binding domain, single chain fusion proteins of this disclosure, in the absence of a cross-linker (i.e., a molecule that binds to two or more SMIP molecules, such as an anti-IgG antibody), induce a robust calcium flux in T cells. Similar results were obtained with molecule formats expressing the BC3 binding domain, as well as when primed T cells were used.

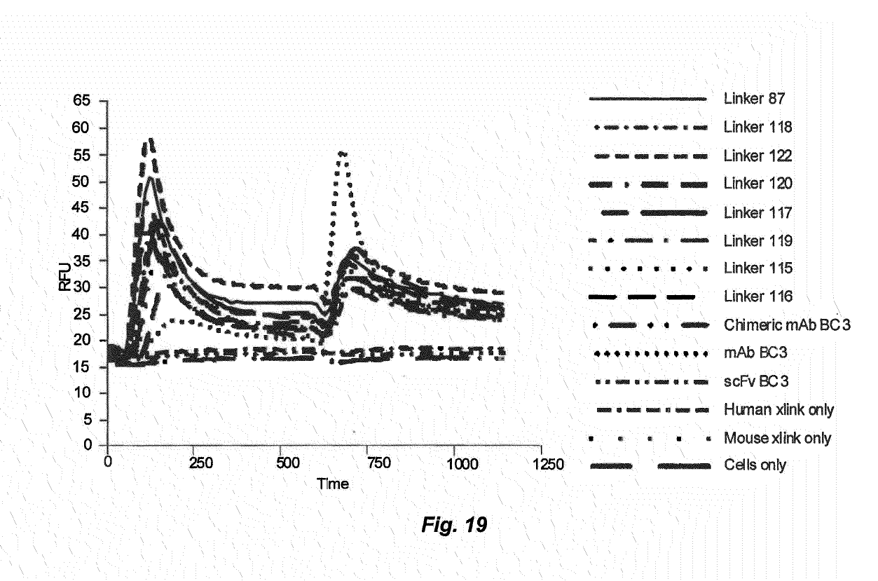

FIG. 19 shows the effect of different hinges on the level of calcium flux caused by single chain fusion proteins having the BC3 binding domain. In this case, the fusion proteins and controls were added at 20 seconds and cross-linkers were added at 600 seconds. The fusion protein with the shortest hinge (Linker 122, derived from an IgA2 hinge) caused greatest calcium flux, while the fusion proteins having longer hinges (Linkers 115 and 116, derived from an IgE C_{H2} and UBA, respectively) induced a lower level calcium flux. But, in all cases the single chain fusion proteins having the BC3 binding domain caused a greater increase in calcium flux than antibodies. The hinge, therefore, may be adjusted to modulate the calcium flux as needed.

### Example 6

### In Vitro Assessment of Anti-Mouse TCR/CD3 Molecules

### Isolation of Mouse Splenocytes

Under aseptic conditions, spleens were excised and large pieces of fat and tissue were removed. In a tissue culture hood, spleens were placed into a small dish with 5 mL of sterile 1×PBS and then ground between two single-sided frosted glass slides. During this process, slides were held at an angle over the Petri dish to allow cells and fluid to run back into the dish. This step was completed when the splenic capsule lost all red color. The cell suspension in the Petri dish was transferred to a 15 mL conical tube and vortexed to break up clumps of cells. The tube then was filled with an additional 12 mL of sterile 1×PBS, stood upright and contents were allowed to settle for 5 minutes. The supernatant was transferred to a second 15 mL conical tube, leaving the settled debris undisturbed in the first tube. The cells were then harvested at 1500 rpm for 5 minutes at room temperature. The supernatant was removed and the cell pellet was suspended in 4 mL of ACK Red Blood Cell Lysing Buffer (Quality Biologics, catalogue No. 118-156-101) and incubated at room temperature for 5 minutes. The conical tube was then filled with RPMI Complete media (RPMI-1640 containing 10% FBS, 100 U/mL penicillin, 100 μg/mL Streptomycin, and 2 mM L-glutamine). The cell suspension was filtered through a cell strainer and transferred to another 15 mL conical tube. Cells were washed three times with complete RPMI and then counted using a hemacytometer.

Labeling Mouse Splenocytes with Carboxyfluorescein Succinimidyl Ester (CFSE)

The density of mouse splenocytes was adjusted to 1×10⁶/mL in sterile PBS. The cells were distributed into 50 mL conical tubes with no more than 25 mL (25×10⁶ cells) per tube. The cells were labeled with CFSE using the CELLTRACE™ CFSE Cell Proliferation Kit from Molecular Probes (catalogue No. C34554), after optimizing conditions for use with human PBMC and mouse splenocytes. A 5 mM solution of CFSE in tissue culture grade DMSO was prepared immediately before use by adding 18 μL of high grade DMSO (Component B of kit) to a vial containing 50 μg of lyophilized CFSE (Component A of kit). Because CFSE is light sensitive, care was taken during the reagent preparation and subsequent cell labeling procedures to protect the reagent from light. The CFSE solution was added to the PBMC cell suspensions at a final concentration of 50 nM CFSE. The caps of the tubes were placed loosely over the tubes containing the cell suspensions to allow for gas exchange, and the tubes were placed in a 37° C., 5% CO₂ incubator for 15 minutes. The cell labeling reaction was quenched by filling the tubes with RPMI Complete (RPMI-1640 containing 10% FBS, 100 U/mL penicillin, 100 μg/mL Streptomycin, and 2 mM L-glutamine) as serum quenches the labeling reaction. The cells were spun at 1500 rpm for 7 minutes at room temperature. The supernatant was aspirated from each tube and the cells were re-suspended in RPMI Complete. The cells were counted (losses of up to 25% of the input are common) and adjusted in RPMI Complete to the desired density for use in assays.

### ConA Blast

Splenocytes were isolated from a BALB/c mouse as previously described and suspended at a concentration of 2×10⁶ cells/mL in complete RPMI media (RPMI, 10% FBS, 2 mM L-glutamine, sodium pyruvate, non-essential amino acids, pen/strep, and 1% BME) and stimulated with 1 ug/mL of concanavalin A (Sigma) for 3 days. After 3 days, cells are washed twice with complete RPMI and re-plated in a new flask with no stimulation for 4 days. At the end of this 4 day rest period, cells were harvested and labeled with CFSE as previously described.

At this time, a second spleen was harvested from a BALB/c mouse and the splenocytes isolated. These freshly isolated splenocytes were used as accessory cells during the restimulation phase of the experiment. To prepare the accessory cell population, T cells (CD5′ cells) were magnetically separated from the fresh splenocytes using the MACS technology from Miltenyi. Supermagnetic beads coated with anti-CD5 antibody (Miltenyi, catalogue No. 130-049-301) were incubated with the freshly isolated splenocytes according to the manufacturer's protocol. The cell and bead mixture was then applied a column (Miltenyi, catalogue No. 130-042-401) containing a matrix that forms a magnetic field when placed in a MACS Separator (Miltenyi, catalogue No. 130-042-301), a strong permanent magnet. The CD5′ cells (T cells) were retained in the column and the untouched accessory cells flowed through. The negatively selected accessory cells were treated with mitomycin C (as previously described) to inhibit proliferation.

Both CFSE-labeled ConA blast and MMC treated accessory cells were resuspended in complete media at 2×10⁶/mL. 0.5 mL of each cell population was added to a 48-well tissue culture plate along with the indicated treatments. 50 μL of supernatant was harvested at 24 hrs after stimulation and the cells and remaining supernatant were harvested on Day 4 post-restimulation. Cells were stained with fluorescently tagged antibodies against CD5 (45-0051, eBioscience) and CD25 (25-0251, eBioscience), run through a flow cytometer (LSR11, Becton Dickenson) and analyzed with FlowJo software (TreeStar). The gating strategy was as follows: cells that fell within a FSC:SSC lymphocyte gate were analyzed for CD5 expression, cells that subsequently fell within the CD5+ gate were then analyzed for CFSE dilution and CD25 upregulation. Cells that were CD5+, CFSE^{lo} and CD25^{hi} were considered activated T cells. Supernatant samples were analyzed for the presence of cytokines and chemokines using a 22 analyte, Linco-plex, Luminex-based detection kit (Linco Research) following the manufacturer's protocol with the following modifications: Analyte beads, detection antibodies, and streptavidin-PE stock solutions were dilutedl:2 prior to use in the assay. The 22 analytes detected by the kit were: MIP-1α, GMCSF, MCP-1, KC, RANTES, IFNγ, IL-1B, 1L-1a, G-CSF, IP-10, IL-2, IL-4, IL-5, IL-6, IL-7, IL-10, IL-12, TNFα, IL-9, IL-13, IL-15, and IL-17.

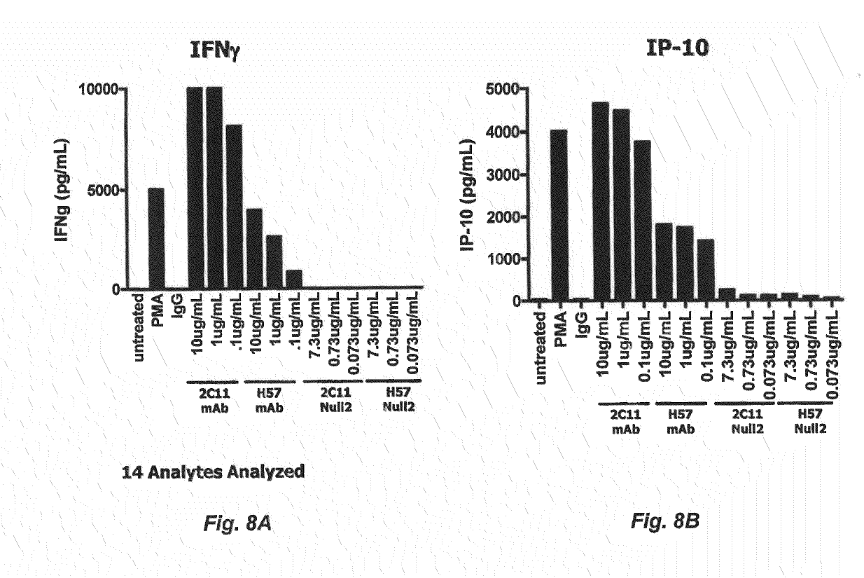

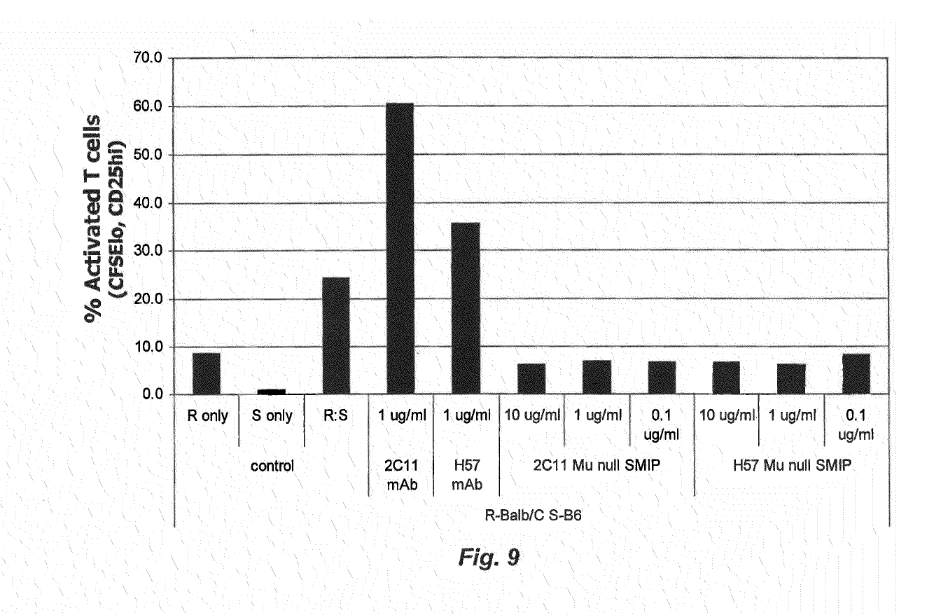

Both H57-457 and 145-2C11 monoclonal antibodies, but not H57 Null2 or 2C11 Null2 SMIP, induced cytokine release of ConA-primed T cells. The results of the release of exemplary cytokines, IFNγ and IP-10, following the treatment of ConA-primed T cells are shown in FIGS. 8A and 8B. In addition, both H57 Null2 (same as “H57 Mu Null” in FIG. 9) and 2C11 Null2 SMIPs (same as “2C11 Mu null SMIP” in FIG. 9), but not H57-457 or 145-2C11 monoclonal antibody, blocked T cell response to antigen (see, FIG. 9). Similar results were obtained when the release of other cytokines were measured.

### Example 7

### In Vivo Studies of Exemplary Anti-TCR SMIPs

Twelve-week old female BALB/c mice (Harlan) were divided into groups of six and injected via the lateral tail vein with 7.3 μg, 37 μg, 75 vg, or 185 μg H57 Null2 SMIP, 5 μg (highest tolerable dose) of H57 mAb, 250 μg of IgG2a isotype control (molar equivalent of the highest SMIP dose), or 200 μL of PBS. All injection volumes were 200 μL and all injected materials had an endotoxin level below 0.5 EU/mg. Three randomly-selected mice per group were terminated at 24 hours and the remaining three mice per group were terminated at the end of the experiment three days post-injection. Mice were monitored for clinical symptoms of drug-associated toxicities in the form of weight loss and increased clinical score. The scientist evaluating clinical score was blinded to the treatments administered to each group. Scores were assigned based on the following key: 0=normal; 1=Mild Piloerection; 2=Moderate Piloerection and/or Ocular Inflammation or Irritation; 3=Hunched Posture/Listlessness; 4=Moribund. All mice were bled at 2 hours post-injection and at their terminal timepoint. Spleens and inguinal lymph nodes were harvested at the terminal timepoints. Sera samples were analyzed for the presence of cytokines and chemokines using a custom 14-plex Luminex-based detection kit from Millipore (Milliplex series), following the manufacturer's protocol, with the following modifications: Analyte beads, detection antibodies, and streptavidin-PE stock solutions were diluted1:2 prior to use in the assay. In addition, serum samples were run neat (compared to recommended 1:2 dilution). The 14 analytes detected by the kit were: G-CSF, GM-CSF, IL-2, IL-4, IL-5, IL-6, IL-10, IL-13, IL-17, IP-10, KC, MCP1, IFNγ, and TNFα. Cell suspensions from spleen and lymph nodes were stained with antibodies against CD5 (eBioscience, catalogue No. 45-0051) and mouse IgG2a (BDBiosciences, catalogue No. 553390) for the determination of the percentage of T cells in these two organs that were coated with the SMIP.

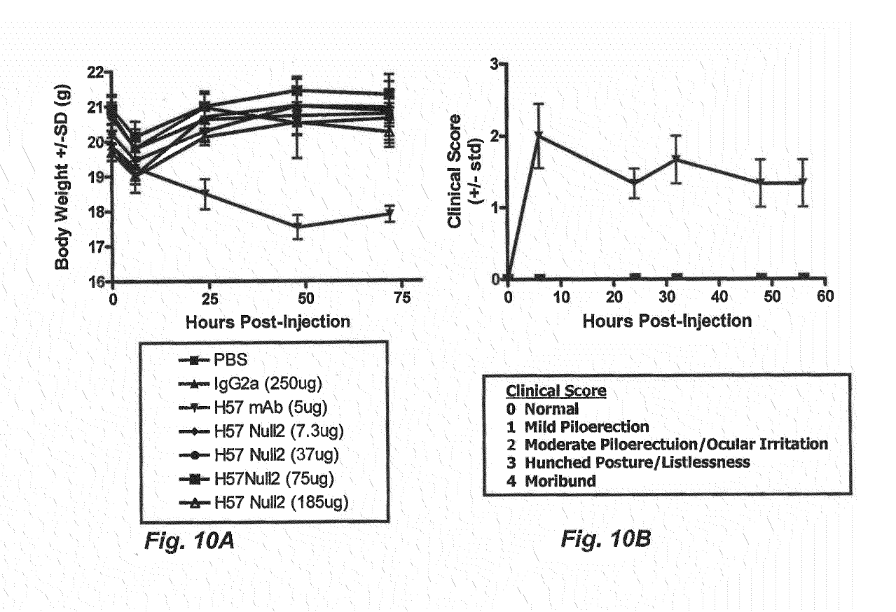

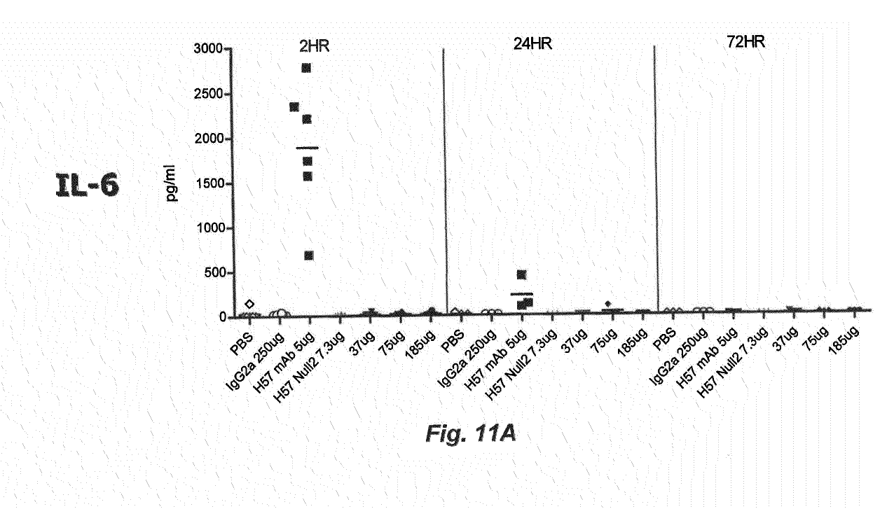

FIG. 10A shows that intravenous administration of H57 Null2 SMIP did not cause loss of body weight. FIG. 10B shows that such treatment did not caused an increase in clinical score, either. These results demonstrate that this Null2 SMIP has the desired safety profile.

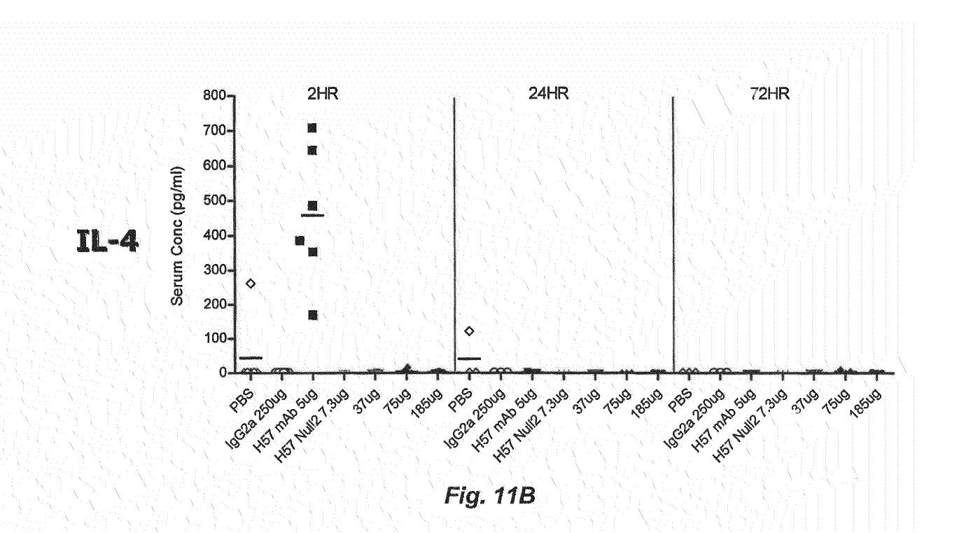

FIG. 11 shows that intravenous administration of H57 Null2 SMIP did not induce cytokine storm in normal BALB/c mice, in contrast to the parental antibody. Two representative cytokines, IL-6 and IL-4 from the 14 analyte panel are shown.

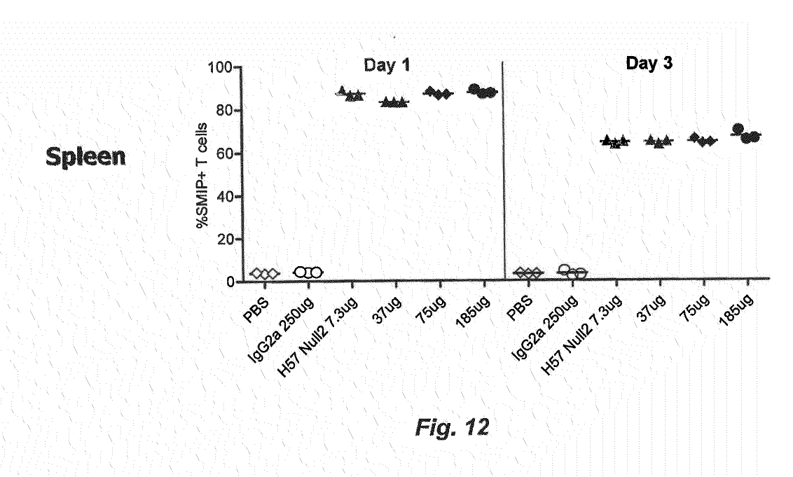

FIG. 12 shows that H57 Null2 SMIP coated T cells were detected in the spleen after intravenous administration of H57 Null2 SMIP.

### Example 8

### Fusion Protein Inhibits Acute Graft Versus Host Disease In Vivo

To determine if surrogate molecules are efficacious in an acute graft versus host disease (aGVHD) mouse model, mice were treated with exemplary fusion proteins and then monitored for weight loss, donor:host lymphocyte ratio, and cytokine and chemokine production.

aGVHD was induced in female C57BL/6XDBA2 F1 mice (Taconic) by transferring splenocytes from donor female C57BL/6 mice (Taconic). Spleens from donor mice were collected and submerged in cold RPMI containing 10% FBS. The collected spleens were dissociated using sterile, frosted glass slides. The supernatant was collected, spun down, and the cells washed as described previously. Washed splenocytes were then resuspended in sterile PBS at a concentration of 65×10⁶ per 200 μl. Immediately before injection, the splenocyte mixture was passed through a 100 pm cell strainer (BD Falcon) to remove debris and large clumps of cells. 200 μl of the donor splenocyte cell suspension was injected intravenously (IV) through the lateral tail vein of the F 1 recipient mice. For IV injections via the lateral tail vein, mice were exposed briefly to a heat lamp and confined in a plastic mouse restrainer. Injections were administered using a 27.5 gauge needle. Recipient mice had pronounced disease by day 14 after donor cell transfer, and at this time point the experiment was terminated and evaluated. Disease progression was associated with body weight loss and the expansion of donor cells with concomitant loss, due to donor cell-mediated attack, of host cells in the spleen of transferred animals. Serum biomarkers such as IFNγ have also been correlated with disease progression.

For efficacy studies, donor cells were transferred into F1 recipients on Day 0 (D0) of the study as described above. The SMIP, IgG2a control and PBS treatments were administered on D0, D1, D3, D5, D7, D9, and D11 with the experiment being harvested on D14. All treatment injections were administered IV except for the D0 injection which was given via the retro-orbital sinus prior to the donor cell transfer. 100 μg of H57 Null2 SMIP or IgG2a in a 100 μl volume or 100 μl of PBS is given per injection. All proteins used in the in vivo studies had less than 0.5 EU/mg of endotoxin. Mice treated with the immunosuppressant dexamethasone (DEX; Sigma) received 10 mg/kg per day via intraperitoneal injection (IP).

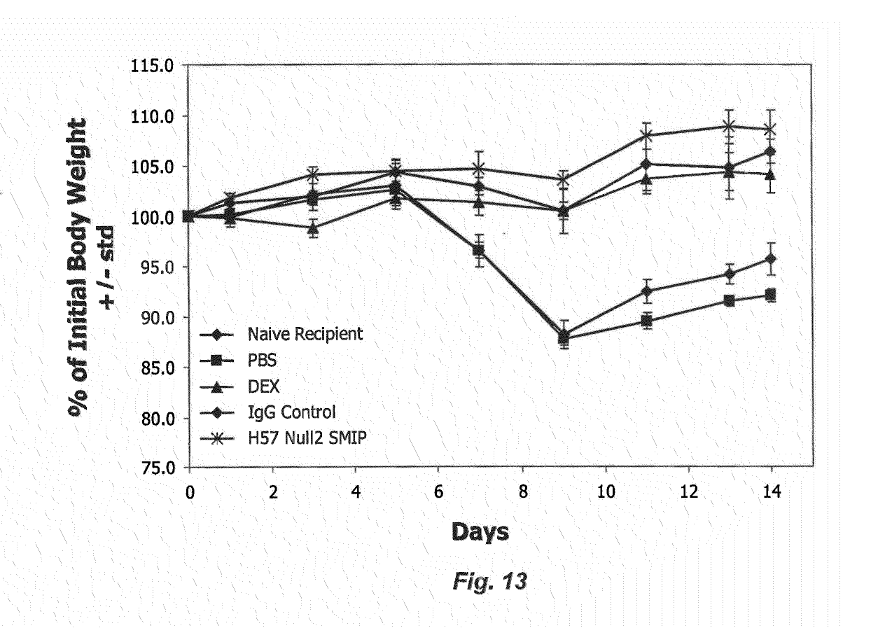

During the course of the experiment, mice were weighed every other day until they started losing weight at which point they were weighed every day. The percentage of initial body weight lost by the recipient mice is depicted in FIG. 13. Administration of H57 Null2 SMIP prevented body weight loss associated with aGVHD disease progression in contrast to mice which received the PBS or IgG2a control treatments.

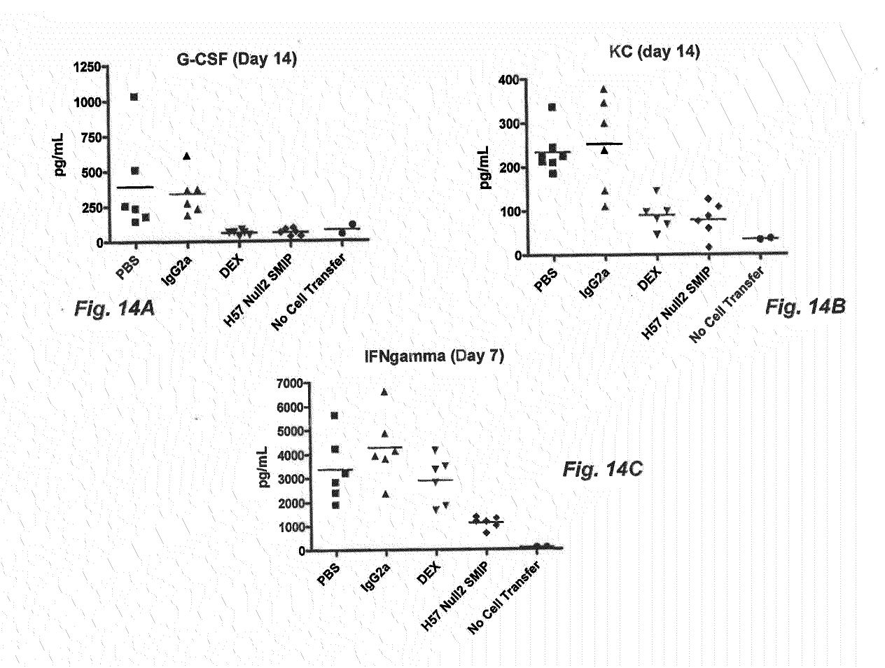

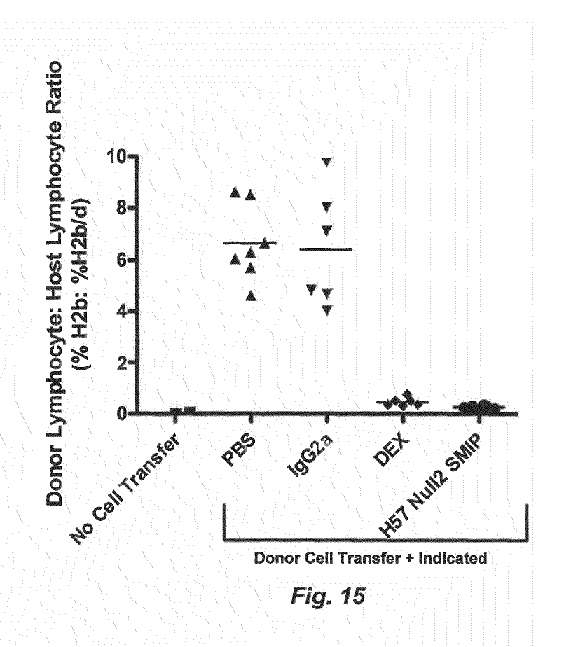

Mice were bled on day 7 for serum biomarker analysis. On day 14, the terminal time point, spleens and blood samples were harvested from each animal. The weights and total cell counts of each spleen were determined. Sera samples were analyzed for the presence of cytokines and chemokines using a custom 14-plex Luminex-based detection kit from Millipore (Milliplex series), following the manufacturer's protocol. The 14 analytes detected by the kit were: G-CSF, GM-CSF, IL-2, IL-4, IL-5, IL-6, IL-10, IL-13, IL-17, IP-10, KC, MCP1, IFNγ, and TNFα. Cytokine and chemokine production was inhibited in mice treated with SMIP, including G-CSF (FIG. 14A), KC (FIG. 14B) and IFNγ (FIG. 14C). These results indicated that administration of SMIP inhibited the cytokine and chemokine production associated with aGVHD, especially the IFNγ production (which is typically highly elevated at day 7 in diseased aGVHD mice). On day 14, splenocytes were isolated as described previously and stained with antibodies against H-2b (donor cells) and H2-d (H2b+, H2-d+ cells were of host origin) for analysis using a LSR11 flow cytometer (BD Biosciences). Mice that received H57 Null2 fusion protein had a donor lymphocyte:host lymphocyte ratio similar to the mice that received DEX and negative control mice that received no donor cells (FIG. 15). These results indicate that fusion proteins of this disclosure inhibit the expansion of donor lymphocytes, which coincides with the decrease in host lymphocytes associated with aGVHD seen in the mice who received the PBS and IgG2a control treatments.

These in vivo studies indicate that fusion proteins of this disclosure inhibit the progression of aGVHD, as evidenced by a lack of donor lymphocyte expansion, inflammatory cytokine and chemokine production, and loss of body weight. Similar efficacy has also been found in preliminary results using a chronic GVHD mouse model.

Experimental models in aGVHD have also been completed to evaluate H57 half null, H57 null2, and 2C11null2. H57 half null and H57 null2 were found to be efficacious with similar results in the parameters examined, despite early release of some cytokines in biomarker studies. The 2C11null2 fusion protein was also efficacious and found to prevent donor cell expansion in the aGVHD model.

### Example 9

### Fusion Proteins with N297A and an Additional Single Alanine Substitution in Igg4 C_{H2} Region Block a T Cell Response to Alloantigen

Human MLR assays were performed as described in Example 2 using the following fusion proteins: OKT3 IgG4-WT-N297A (SEQ ID NO:232), OKT3 IgG4-ALGG-N297A (SEQ ID NO:234), OKT3 IgG4-FAGG-N297A (SEQ ID NO:236), OKT3 IgG4-FLAG-N297A (SEQ ID NO:238), OKT3 IgG4-FLGA-N297A (SEQ ID NO:240), OKT3 IgG4-AA-N297 (SEQ ID NO:91), OKT3 FL and OKT3 mAb.

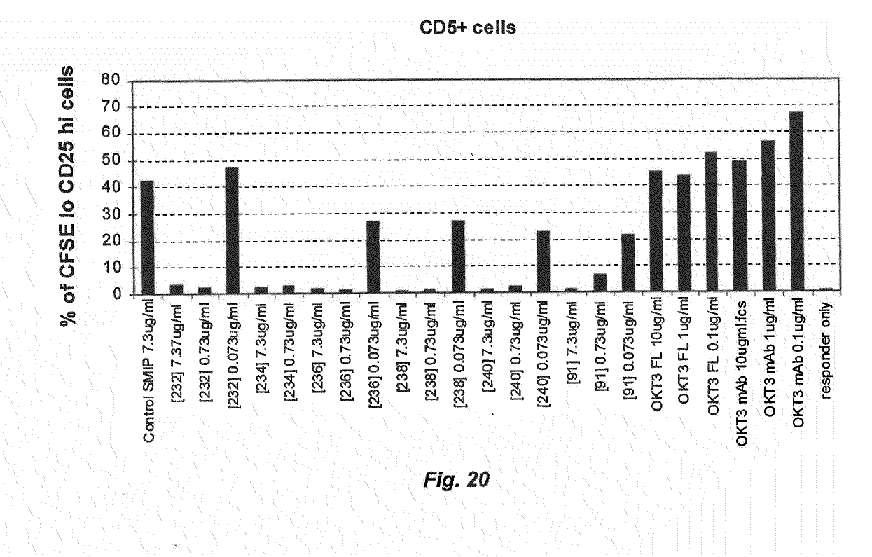

FIG. 20 shows that the OKT3 IgG4 fusion proteins containing (a) only an alanine substitution at N297 or (b) both an alanine substitution at N297 and an additional alanine substitution at position F234, L235, G236 or F237 blocked a T cell response to alloantigen better than known OKT3 mAb and OKT3 ala-ala antibody.

### Example 10

### MLR Reaction can be Influenced by Choice of Hinge Regions

Human MLR assays were performed as described in Example 2 using fusion proteins derived from BC3 IgG1-N297A (SEQ ID NO:80) and containing hinges of various lengths and sequences: Linker 125 derived from UBA (SEQ ID NO:329), Linker 126 derived from an IgE C_{H2} (SEQ ID NO:330), Linker 127 derived from an IgD hinge (SEQ ID NO:331), Linker 128 derived from an IgA2 hinge (SEQ ID NO:332), and Linker 129 derived from an IgG1 hinge (SEQ ID NO:333). The amino acid sequences of the BC3 IgG2-N297A SMIPs containing the above-noted linkers are set forth in SEQ ID NOS:325, 323, 319, 315, and 311, respectively. The nucleotide sequences encoding these BC3 IgG2-N297A SMIPs are set forth in SEQ ID NOS:324, 322, 318, 314, and 310, respectively.

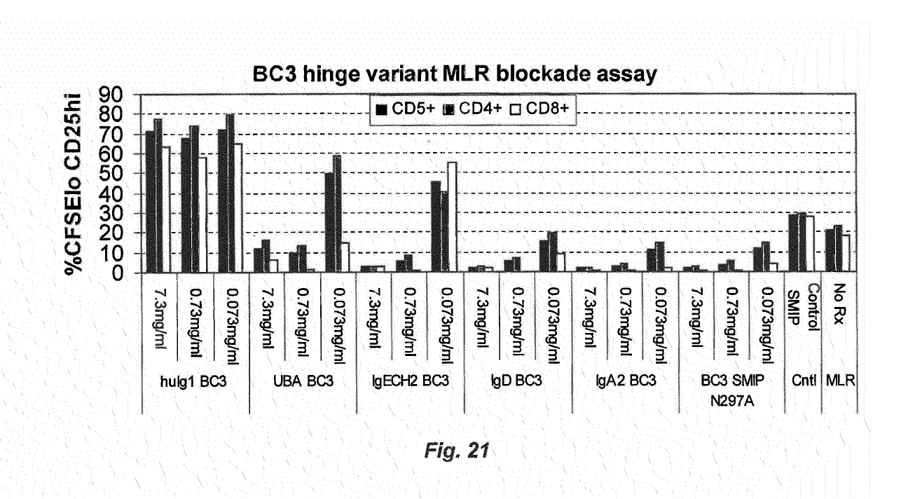

FIG. 21 shows the effect of different hinges on the capability of BC3 IgG1-N297A fusion proteins in blocking a T cell response to alloantigen. It appears that fusion proteins with shorter hinges were more effective in blocking the T cell response. However, in all cases, the single chain fusion proteins having the BC3 binding domain were more effective in blocking the T cell response to alloantigen than HuIg1 BC3 (an antibody molecule that contains the variable region of the BC3 mAb and human IgG1 constant region).

### Example 11

### In Vitro Analysis of Humanized CRIS7 Fusion Proteins

Human MLR assays were performed as described in Example 2 were performed using various humanized Cris7 fusion proteins: humanized Cris7 (VH3-VL1) IgG1-N297A (SEQ ID NO:290), humanized Cris7 (VH3-VL2) IgG1-N297A (SEQ ID NO:295), humanized Cris7 (VH3-VL1) IgG2-AA-N297A (SEQ ID NO:292), humanized Cris7 (VH3-VL2) IgG2-AA-N297A (SEQ ID NO:297), humanized Cris7 (VH3-VL1) IgG4-AA-N297A (SEQ ID NO:293), humanized Cris7 (VH3-VL2) IgG4-AA-N297A (SEQ ID NO:298), chimeric Cris7 IgG1-N297A (SEQ ID NO:265), humanized Cris7 (VH3-VL1) HM1 (SEQ ID NO:294), humanized Cris7 (VH3-VL2) HM1 (SEQ ID NO:299), and chimeric Cris7 HM1 (SEQ ID NO:269).

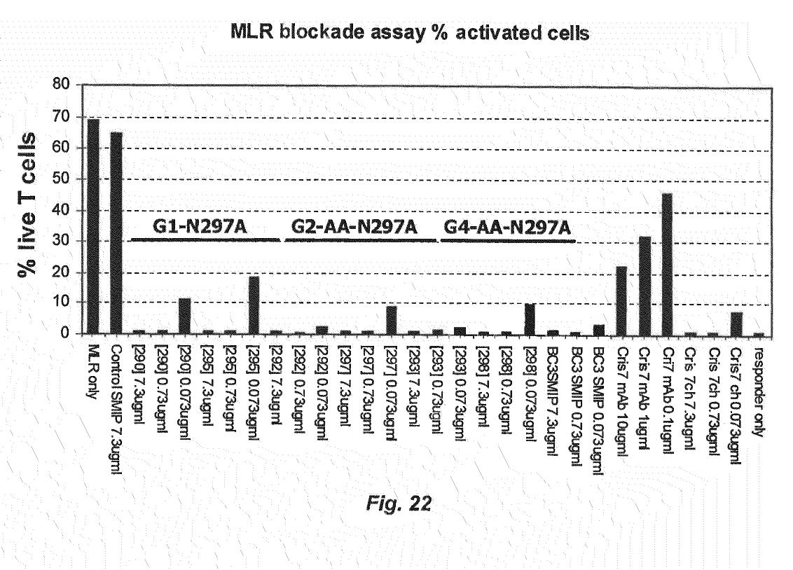

FIG. 22 shows that humanized Cris7 IgG1-N297A, IgG2-AA-N297A and IgG4-AA-N297A fusion proteins and a chimeric Cris7 IgG1-N297A fusion protein blocked a T cell response to alloantigen better than known Cris7 mAb.

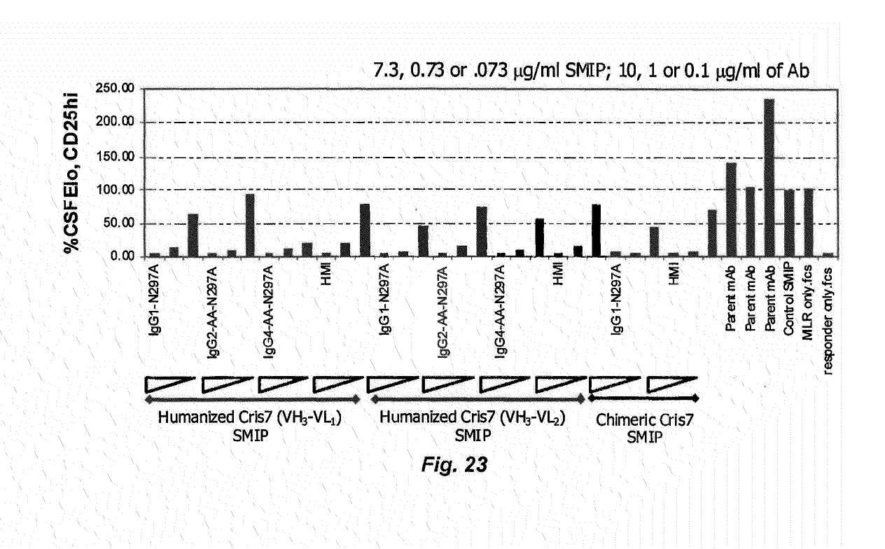

FIG. 23 also shows that humanized Cris7 IgG1-N297A, IgG2-AA-N297A and IgG4-AA-N297A fusion proteins and a chimeric Cris7 IgG1-N297A fusion protein blocked a T cell response to alloantigen better than known Cris7 mAb. In addition, humanized and chimeric Cris7 HM1 fusion proteins also blocked a T cell response to alloantigen better than Cris7 mAb.

Mitogenicity and cytokine release of PHA-primed T cells re-stimulated by humanized Cris7 (VH3-VL1) IgG1-N297A and humanized Cris7 (VH3-VL2) IgG1-N297A fusion proteins were analyzed using the methods described in Example 1. The following cytokines were tested: IL-1b, IL-10, IL-17, IFNγ, TNFα, IL6, MCP-1, IP-10, IL-2 and IL4.

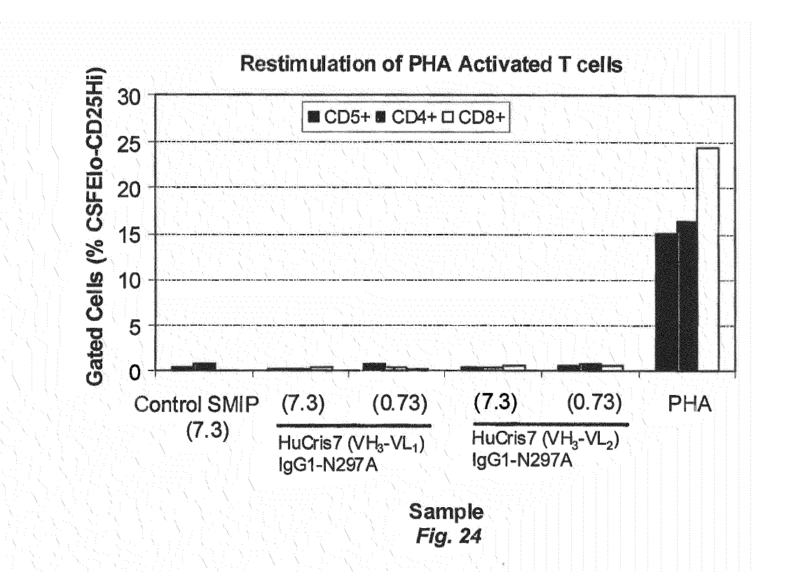

FIG. 24 shows that the humanized Cris7 (VH3-VL1) IgG1-N297A and humanized Cris7 (VH3-VL2) IgG1-N297A fusion proteins did not activate PHA-primer T cells. Similar data were generated with humanized Cris7 (VH3-VL1) IgG2-AA-N297A, humanized Cris7 (VH3-VL2) IgG2-AA-N297A, humanized Cris7 (VH3-VL1) IgG4-AA-N297A, and humanized Cris7 (VH3-VL2) IgG4-AA-N297A fusion proteins.

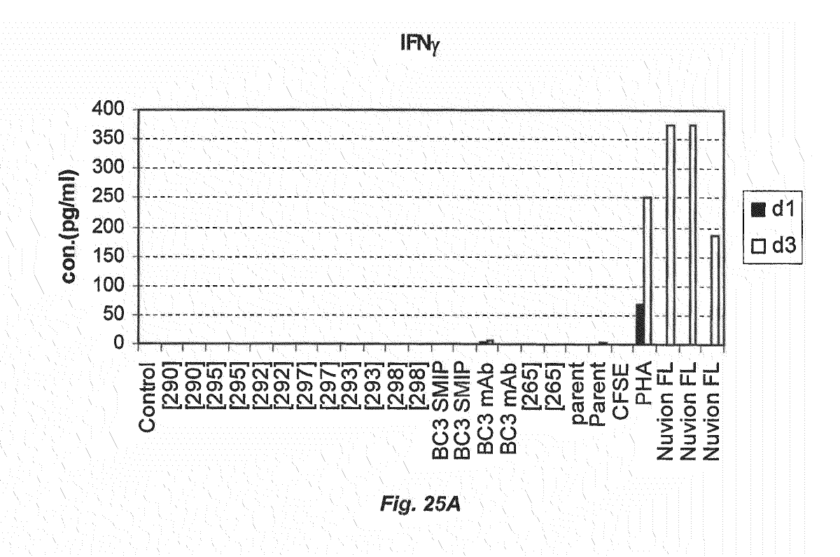

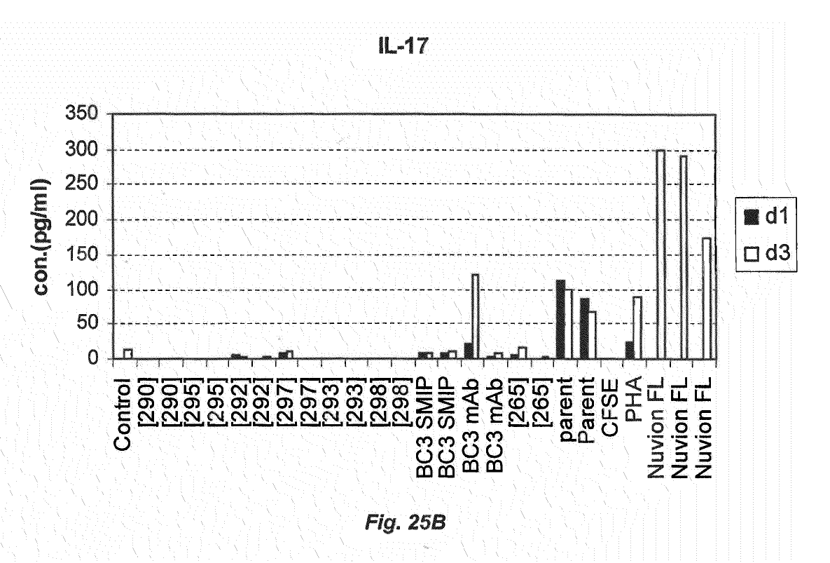

The cytokine release results show that (1) humanized Cris7 IgG1-N297A, humanized Cris7-IgG2-AA-N297A, humanized Cris7-IgG4-AA-N297A and chimeric Cris7 IgG1-N297A fusion proteins were not different from control non-T cell binding SMIP protein, (2) parent Cris7 mAb was comparable to the humanized Cris7 IgG 1-N297A, humanized Cris7-IgG2-AA-N297A, and humanized Cris7-IgG4-AA-N297A fusion proteins except IL-17 (parent Cris7 mAb induced more IL-17 release than the humanized Cris7 fusion proteins), (3) Nuvion FL activated cells to produce IL-10, IFNγ, IL-17, TNFα, and IL-6, and (4) all molecules tested (including control non-T cell binding SMIP) caused secretion of MCP-1 at levels as high as PHA re-stimulation. The results of IFNγ and IL-17 release are shown in FIGS. 25A and 25B, respectively.

Cytokine levels in a primary mitogenicity assay in cynomolgous PBMC in vitro were measured as follows: non-human primate PBMCs from cynomolgus monkeys were isolated as described in Example 1 with the exceptions of using 90% of Lymphocyte Separation Media in PBS 1× (CMF) and preparing the density gradient in 15 ml conical tubes. Cells were resuspended at a concentration of 4×10⁶ cells/ml in RPMI complete media (RPMI-1640 containing 10% human AB serum, 100 U/mL penicillin, 100 μg/mL Streptomycin, and 2 mM L-glutamine) and aliquot to 96 well flat bottom plate at 100u1/well along with indicated treatments. Cells were incubated at 37° C. Supernatants from each well were sampled on day 1, day 2 and day 3, and analyzed for presence of non human primate cytokines using a custom 9-plex Luminex based detection kit from Millipore, following the manufacture's protocol. The 9 analytes detected by the kit were: IL-10, IL-2, IL-4, IL-6, IL-10, IL-17, MCPJ, IFNγ, and TNFα.

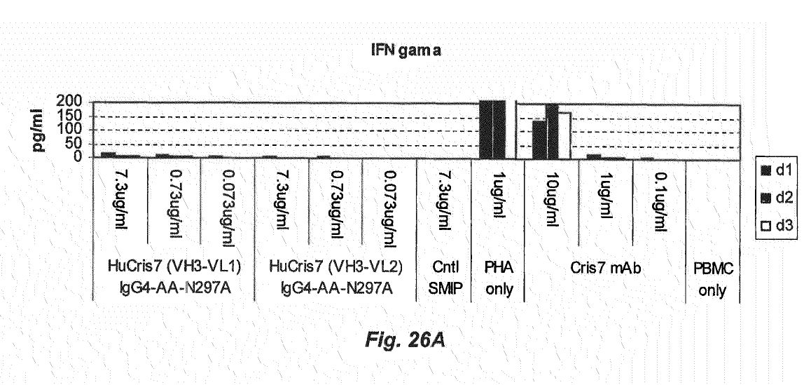

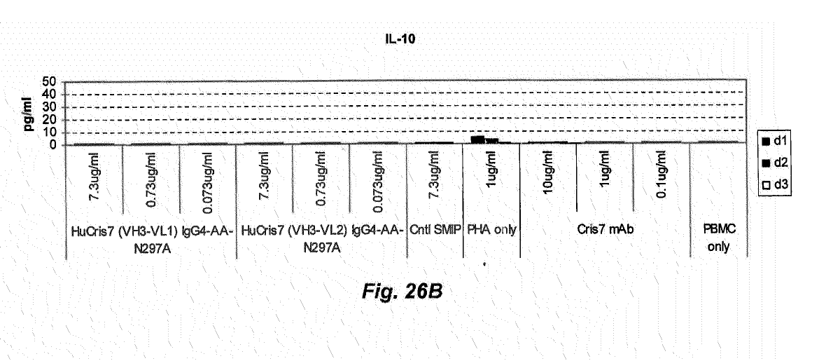

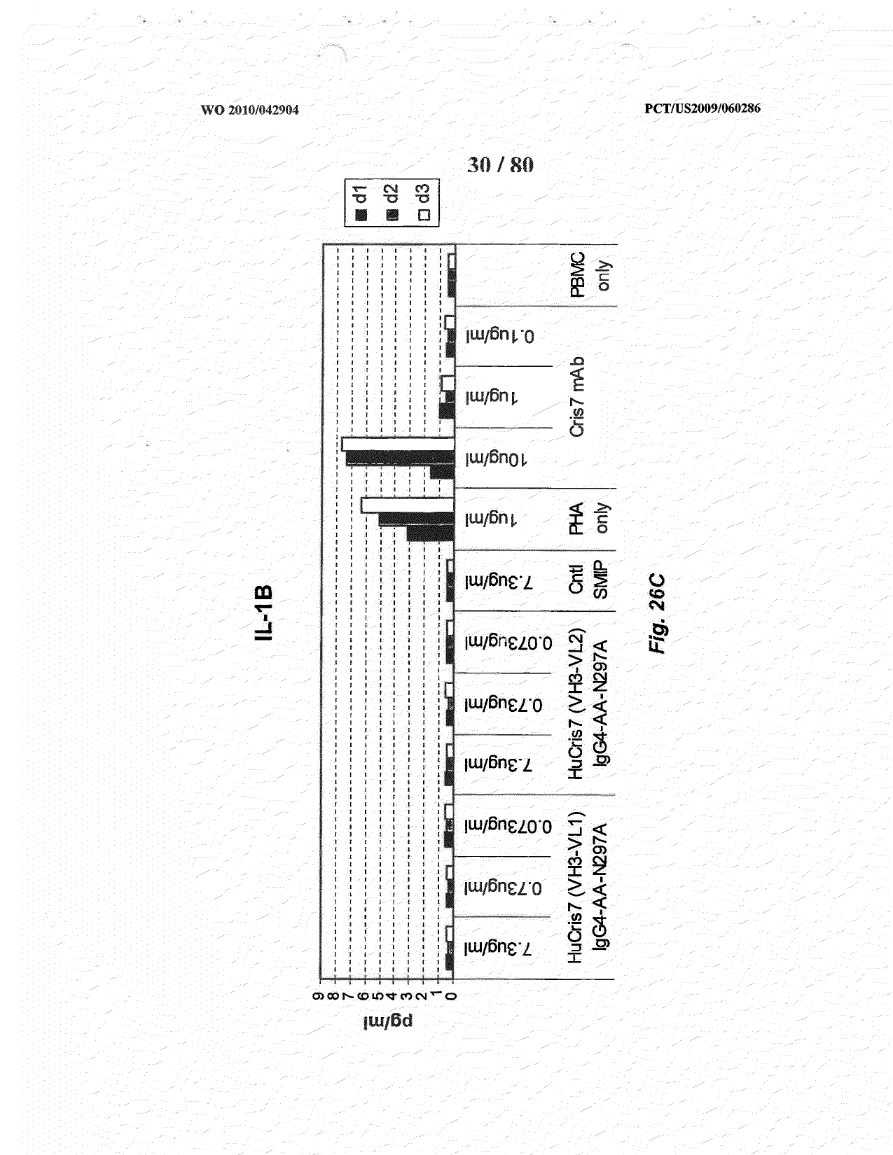

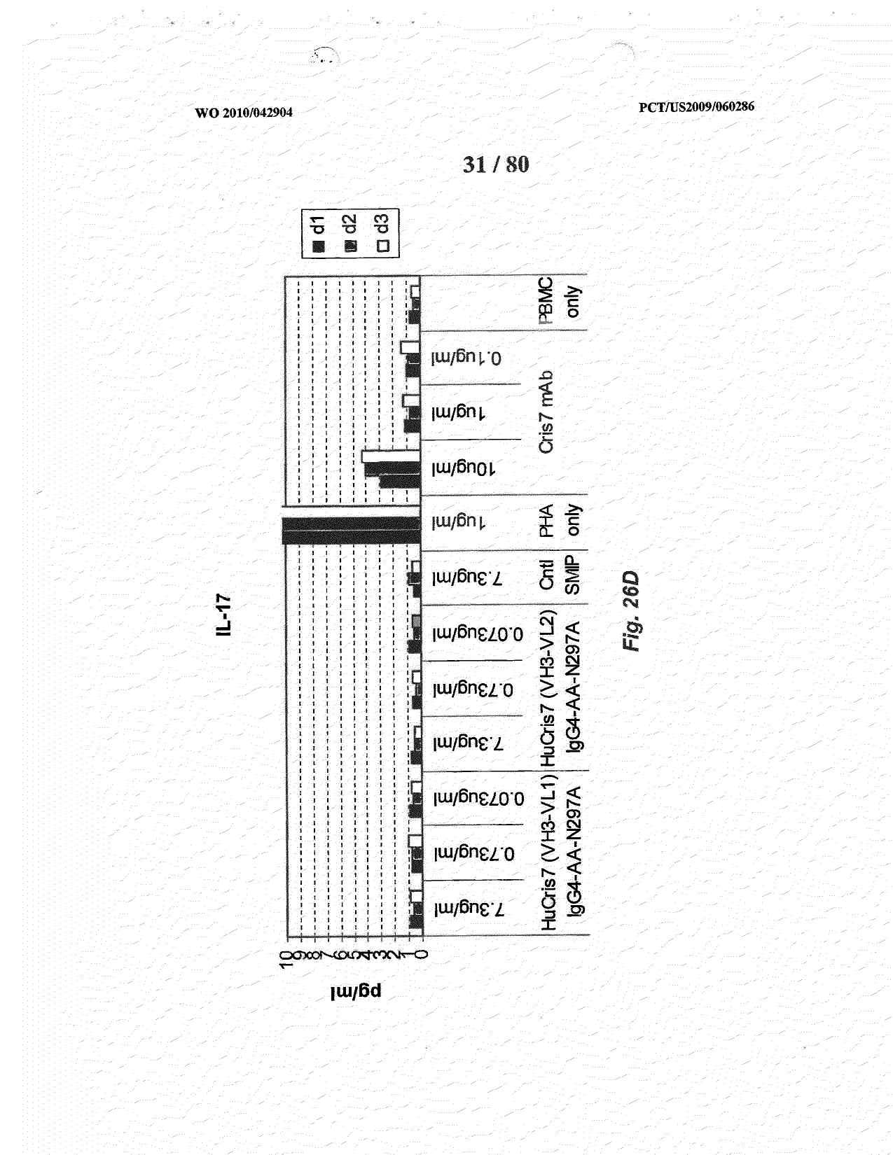

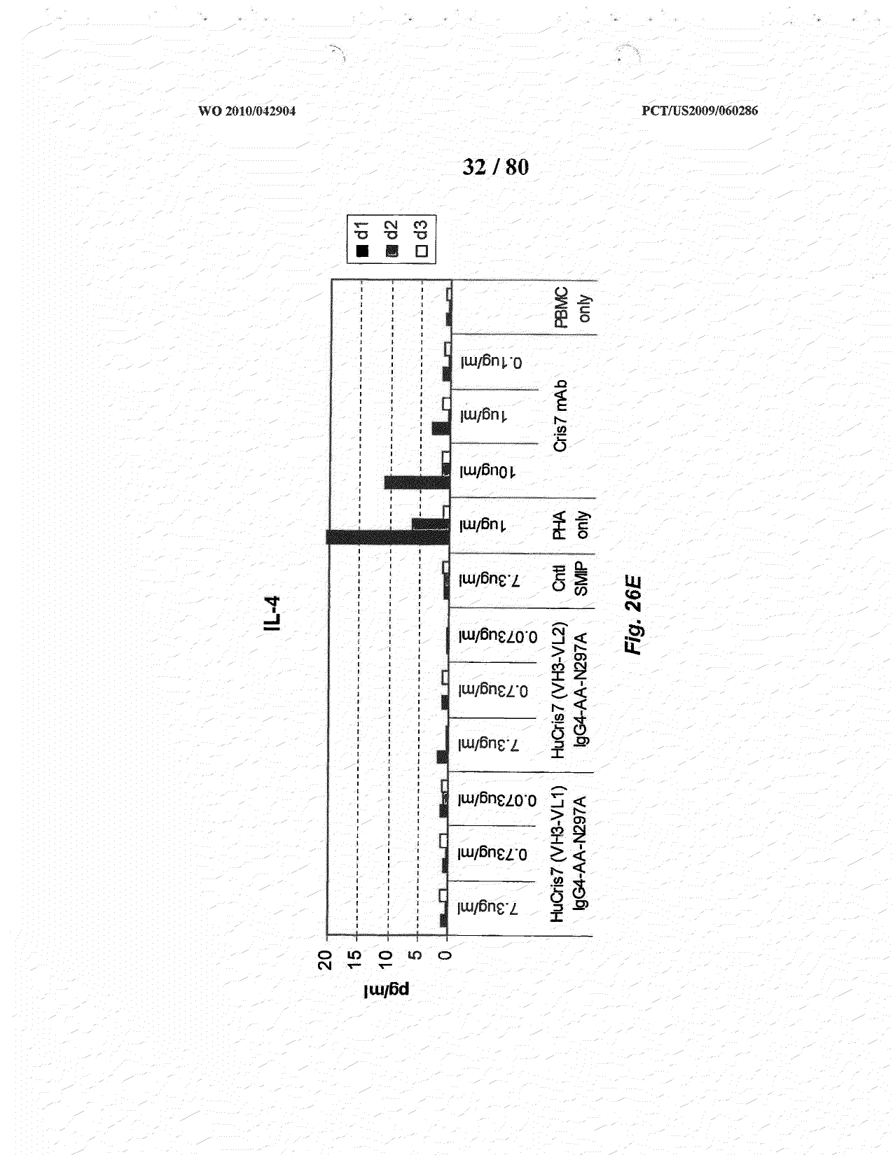

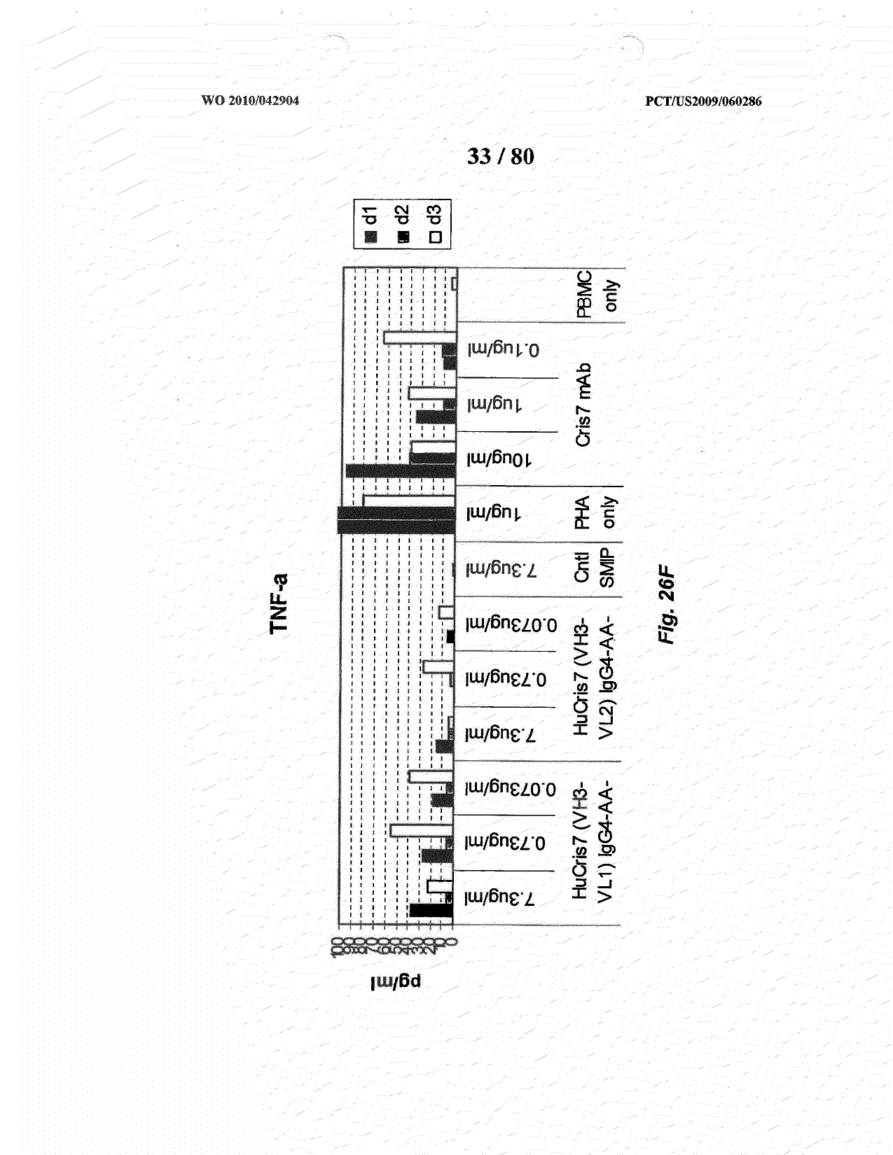

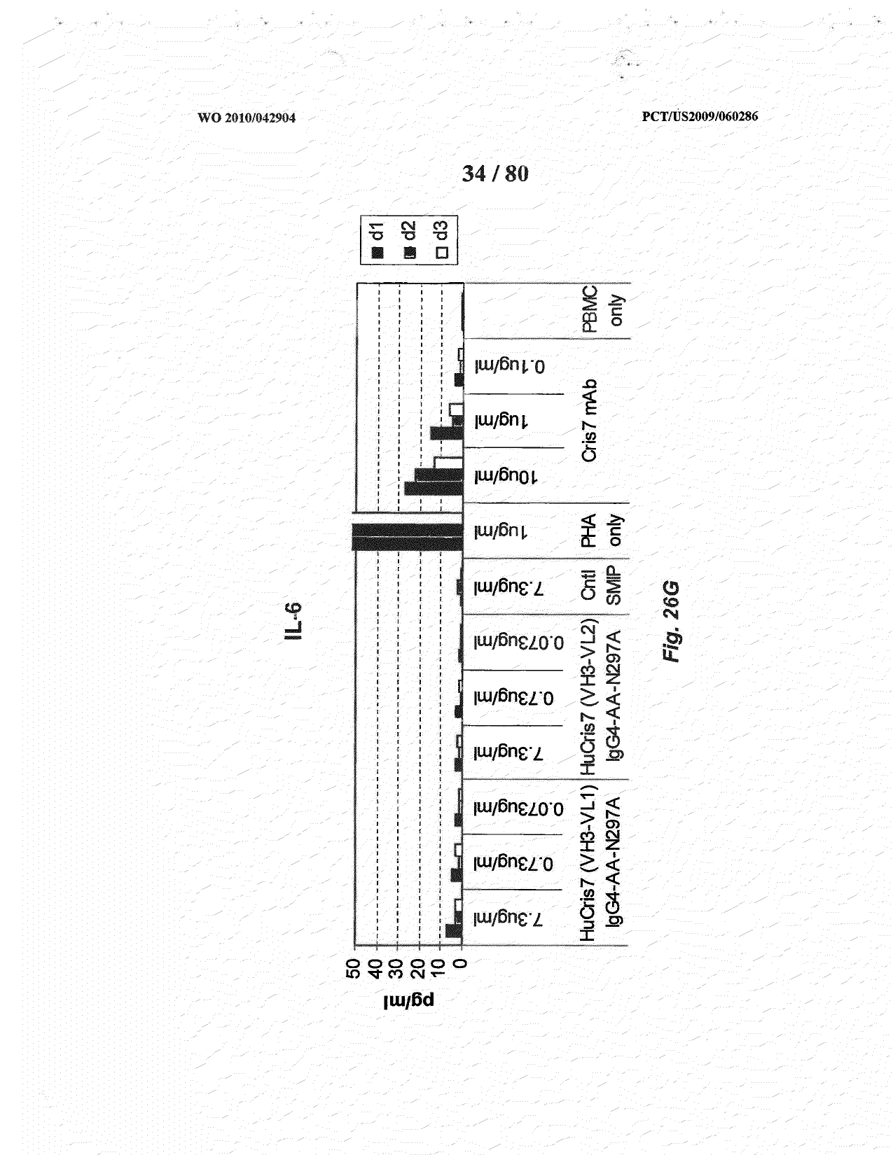

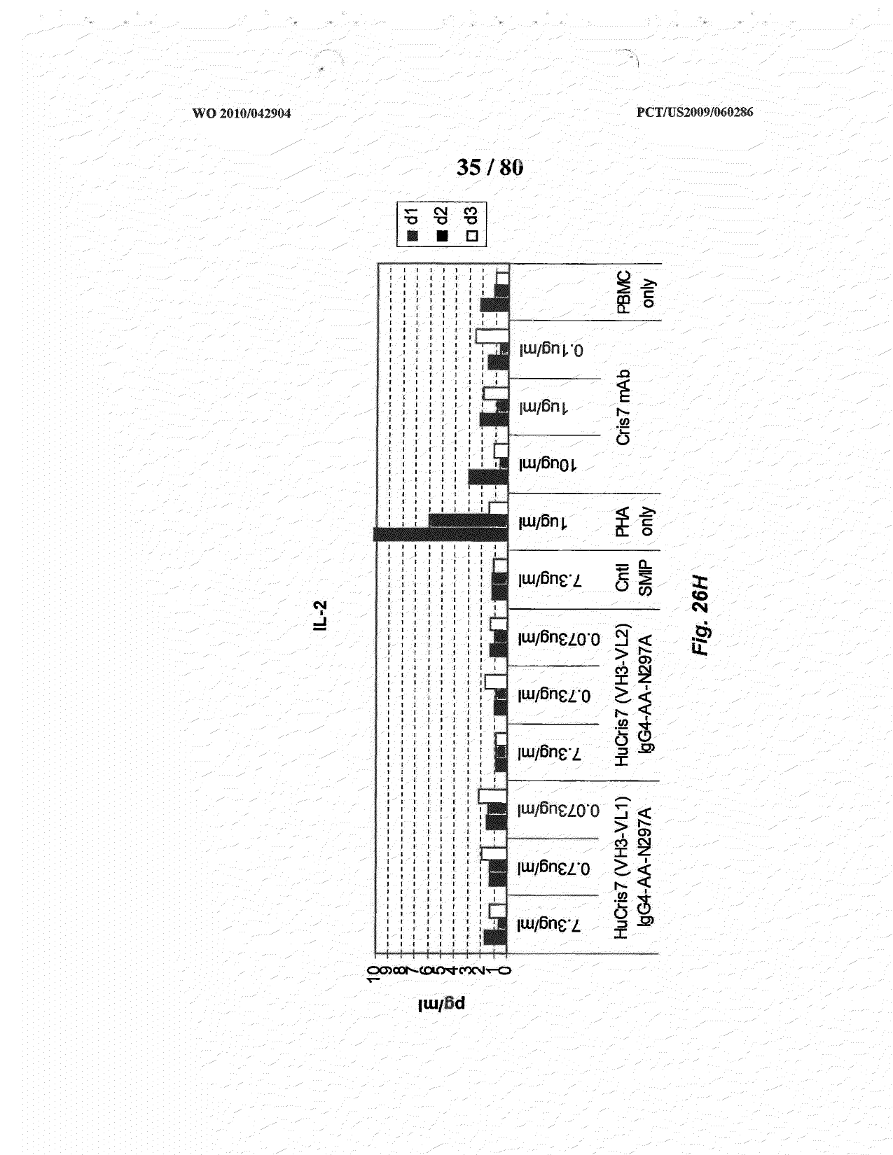

The results (FIGS. 26A-H) show that the humanized Cris7 (VH3-VL1) IgG4-AA-N297A and humanized Cris7 (VH3-VL2) IgG4-AA-N297A fusion proteins induce less release of IFNγ, IL-17, IL-4, TNFα, IL-6 and IL-10 as compared to Cris7 mAb, whereas the levels of IL-1B and IL-2 were comparable after treatments with the humanized Cris7 IgG4-AA-N297A fusion proteins and after treatments with Cris7 mAb.

### Example 12

### Biomarker Study of Exemplary Fusion Proteins Containing H57Binding Domain

Ten-week old female C57BL/6 XDBA2 F1 mice were weight matched and divided into five groups of eight animals per group. Animals were injected IV via the retro-orbital sinus (200 μL of the molar equivalent of 300 μg H57 Null2 SMIP) with IgG2a isotype control, H57 Null2 SMIP (SEQ ID NO:96), H57 ½ Null SMIP (SEQ ID NO:304), H57 HM2 SMIP (SEQ ID NO:306), or 5 μg of H57 mAb. Four mice from each group were euthanized at 24 hours and the remaining four mice per group were euthanized at the end of the experiment three days post-injection. Mice were monitored for clinical symptoms of drug-associated toxicities as previously described. All mice were bled at 2 hours post-injection and at their terminal timepoint. Sera samples were analyzed for the presence of cytokines and chemokines using a custom 14-plex Luminex-based detection kit from Millipore as previously described. In addition to blood collection for serum analysis, an aliquot of blood was collected into whole blood microtainer tubes (containing EDTA) for peripheral blood staining of white blood cells. Briefly, 5 μL of whole blood was added to wells in a 96-well U-bottom plate. 5 μL of Rat Anti-10 μg/ml mouse CD16/CD32 Fc Block (BD Pharmingen) was added and plates incubated at room temperature for 15 minutes, medium speed on a plate shaker. 10 μL of antibody cocktail (or appropriate single stain controls) against CD5 (PE-Cy5), CD19 (FITC, eBioscience) and CD45 (PE, eBioscience) were added for a final dilution of 1:4000. Plates were incubated for an additional 20 minutes at room temperature, light protected, set on a plate shaker at medium speed. 180 μL of 1×BD Pharm Lyse buffer was added and wells mixed thoroughly and allowed to sit at room temperature for 30 minutes. 50 μL of each sample were then analyzed on the BD LSRII High Throughput Sampler (HTS). The gating strategy was as follows: cells that fell within a FSC:SSC lymphocyte gate were analyzed for CD45 expression, cells that subsequently fell within the CD45+ gate were then analyzed for CD5 and CD19 expression. Cells per ml of each cell type were back calculated based on the 50 μL sample collected and dilution factor of 40.

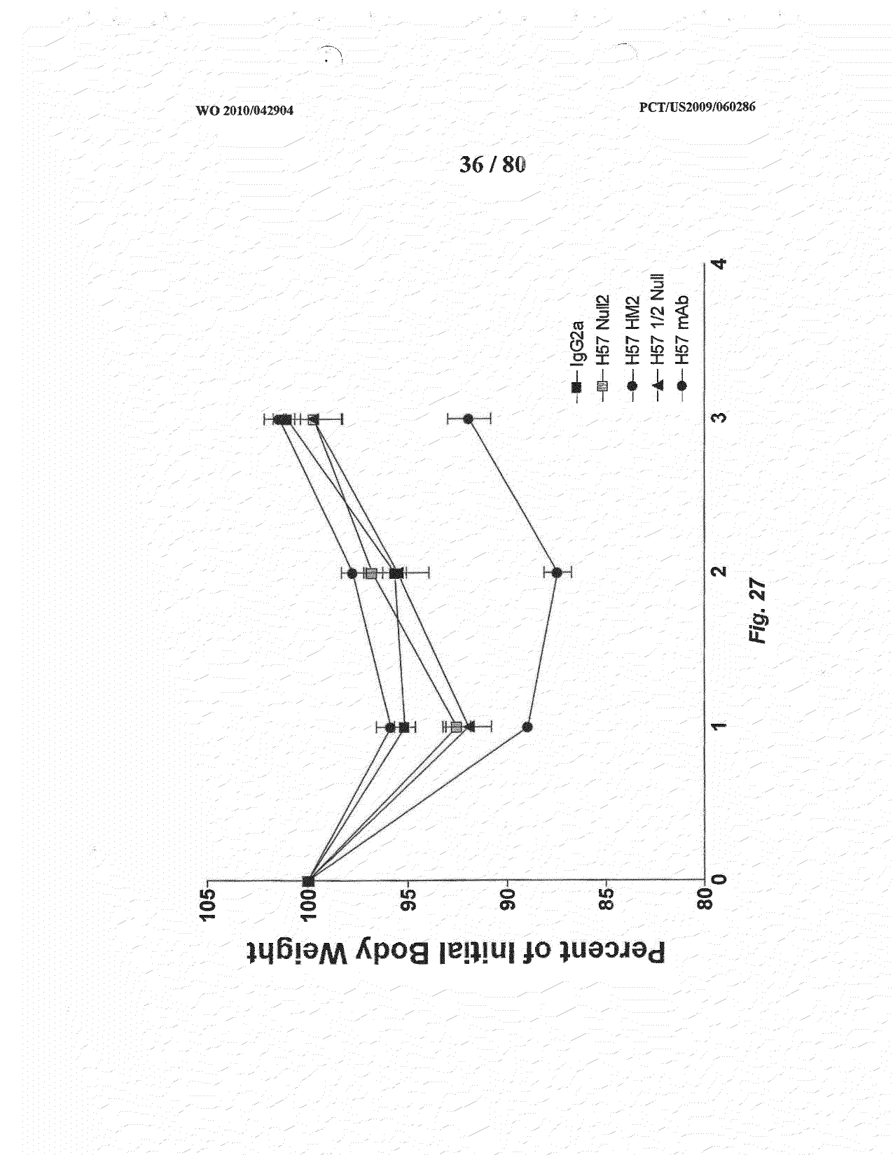

FIG. 27 shows that intravenous administration of H57 Null2, half null and HM2 SMIP proteins did not cause loss of body weight, while intravenous administration of H57 mAb caused loss of body weight. All mice appeared normal without obvious signs of distress between day 0 and day 3.

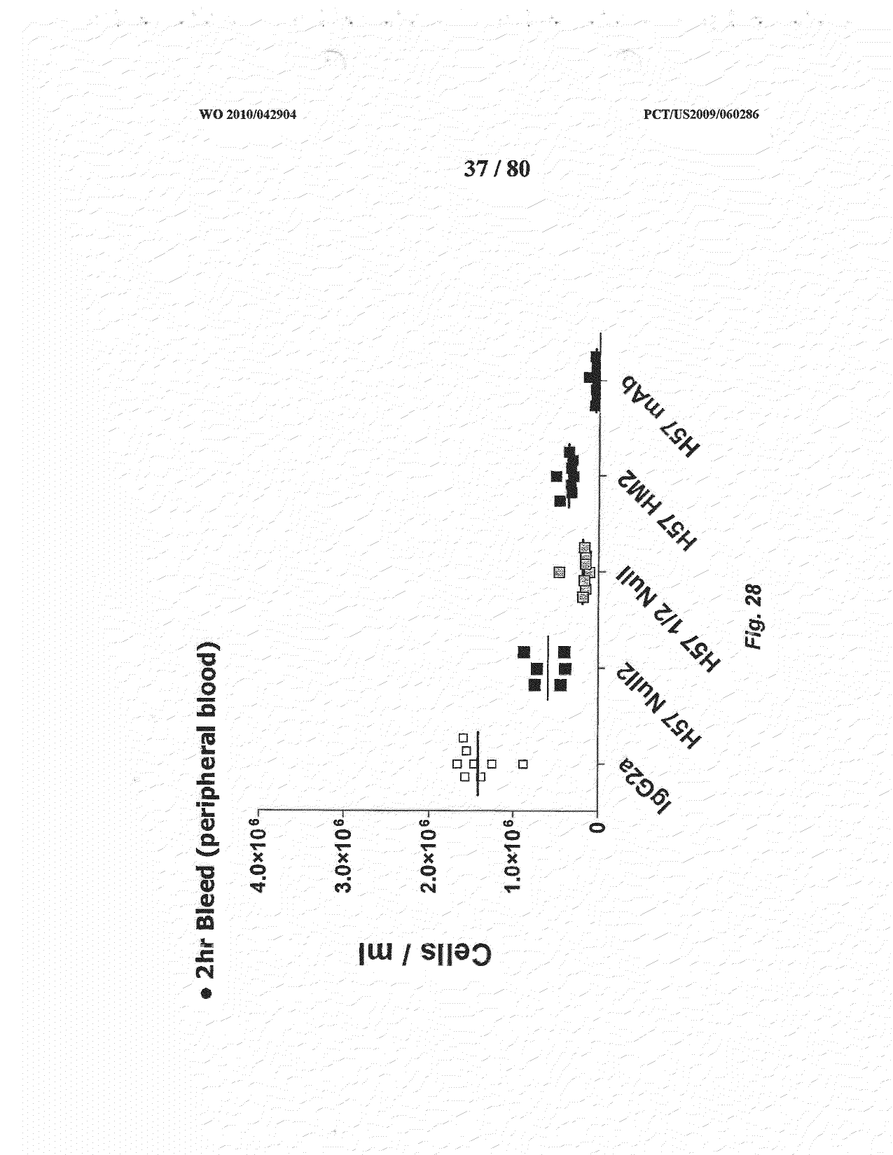

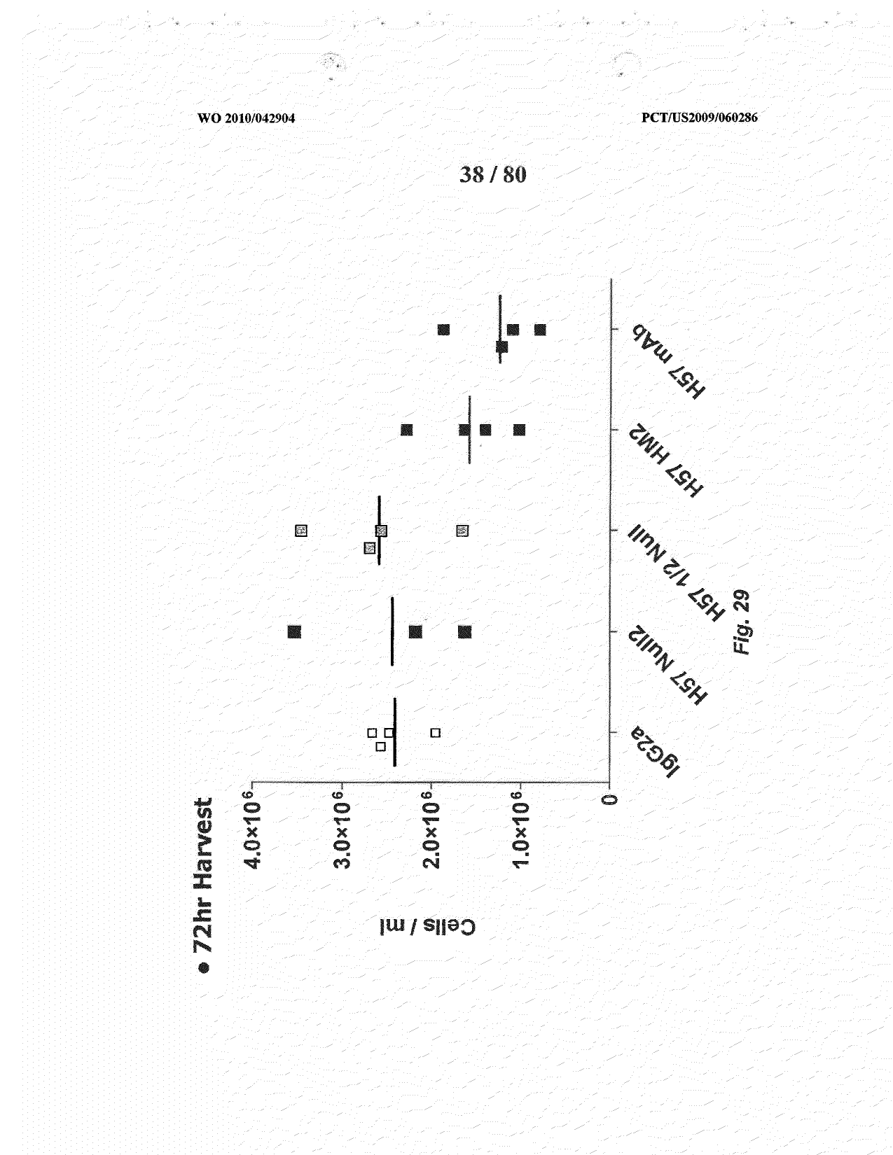

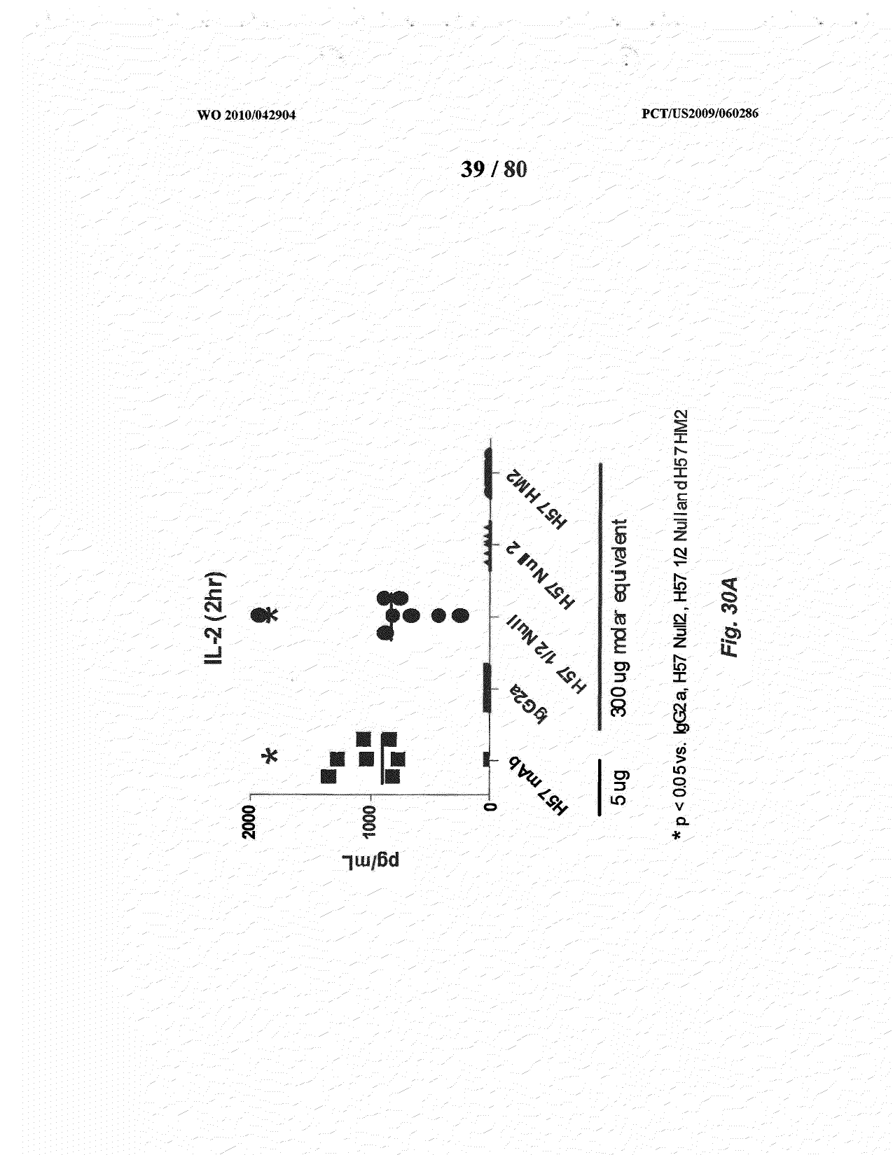

FIG. 28 shows that intravenous administration of H57 Null2, H57 half Null, H57 HM2, or H57 mAb results in a transient decrease in circulating CD5+ T-cells (cells/ml) compared to IgG2a isotype control. Levels of circulating CD5+ T-cells (cells/ml) are not significantly different between groups at 72 hrs after injection (FIG. 29).

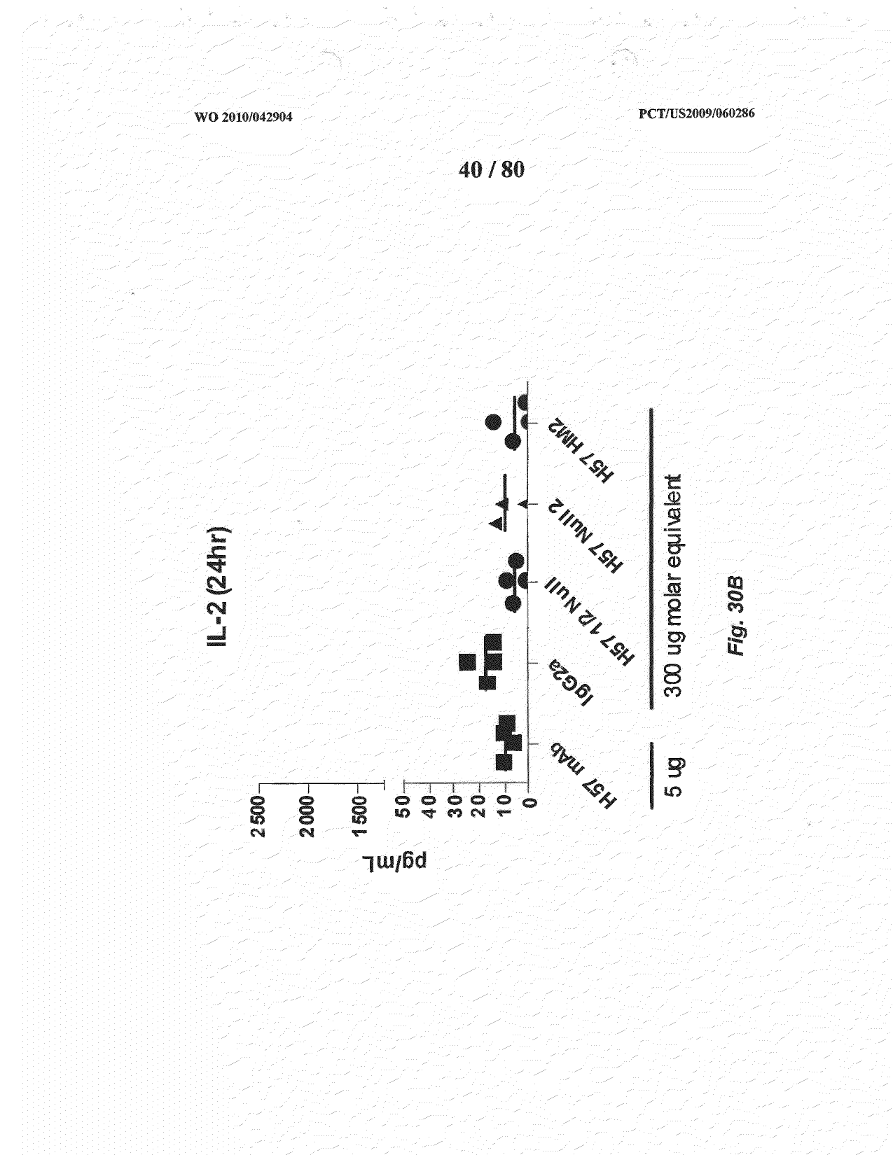

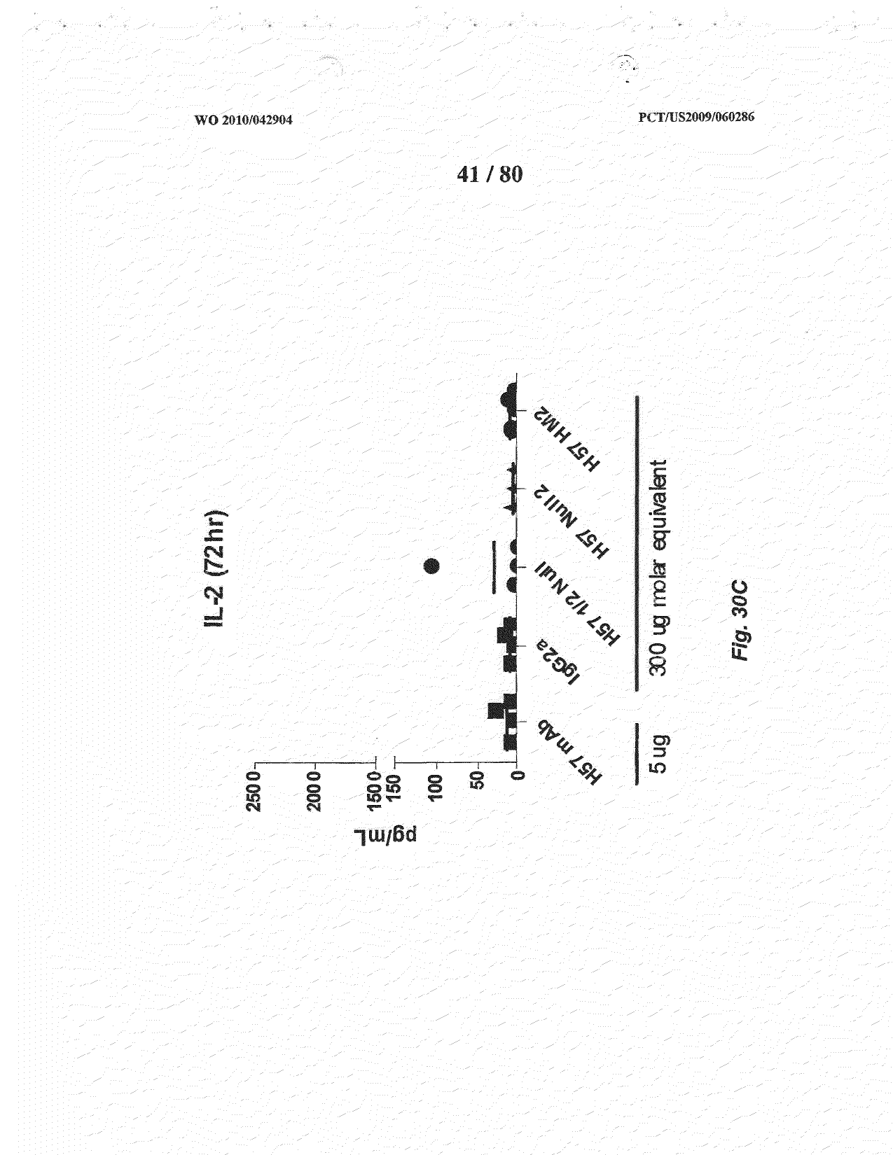

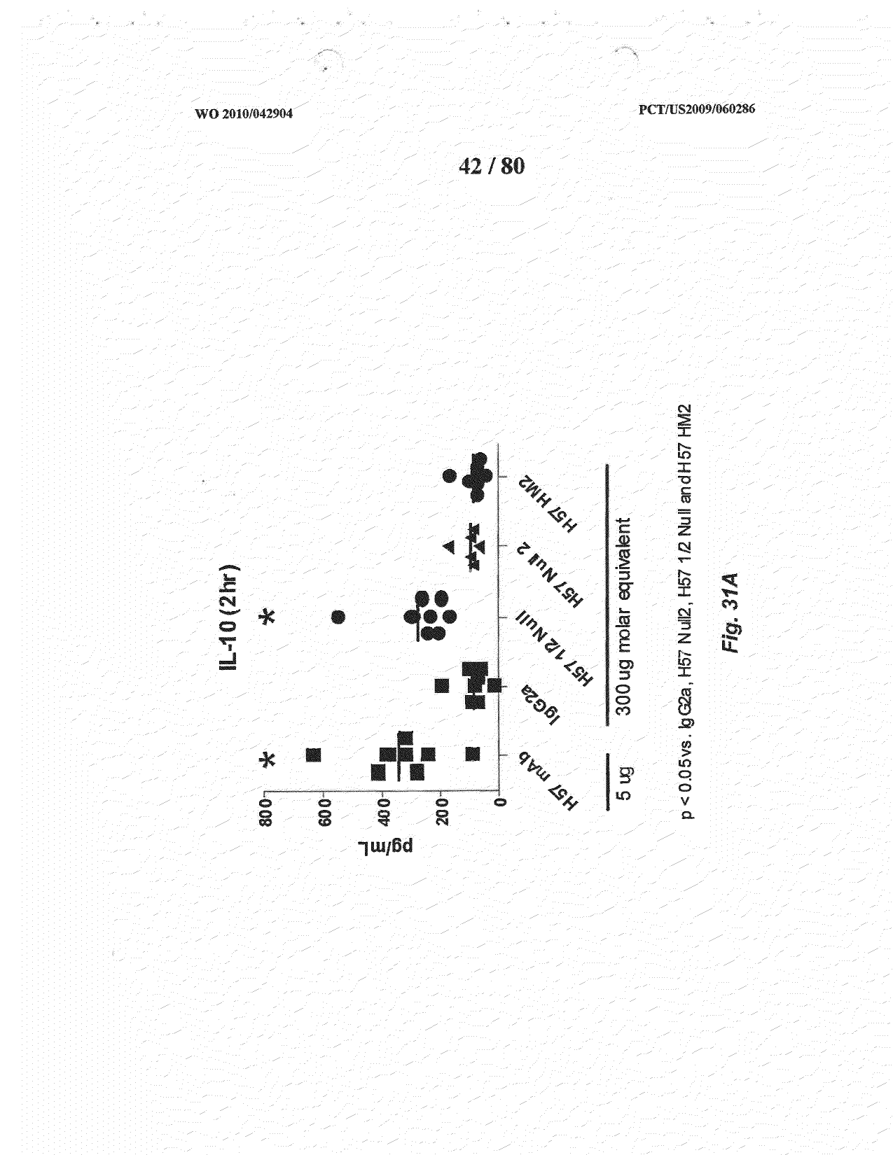

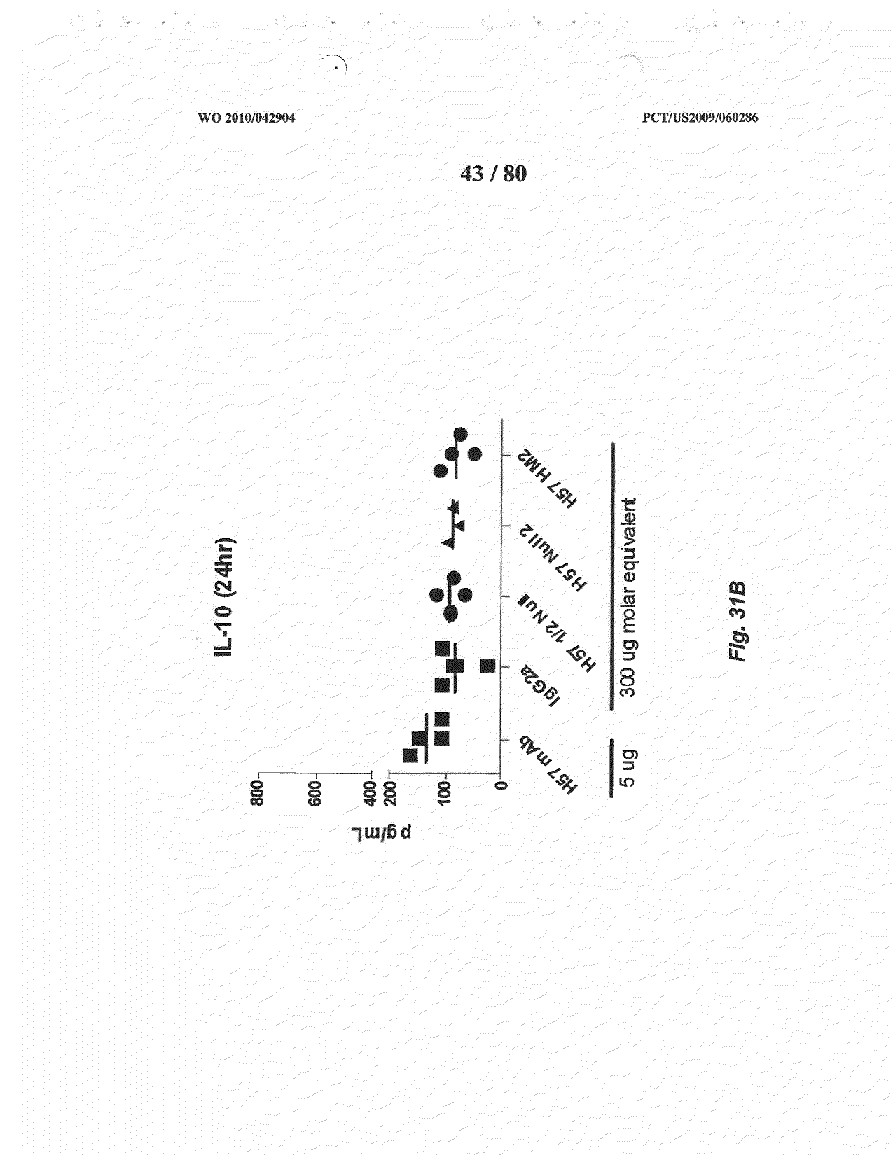

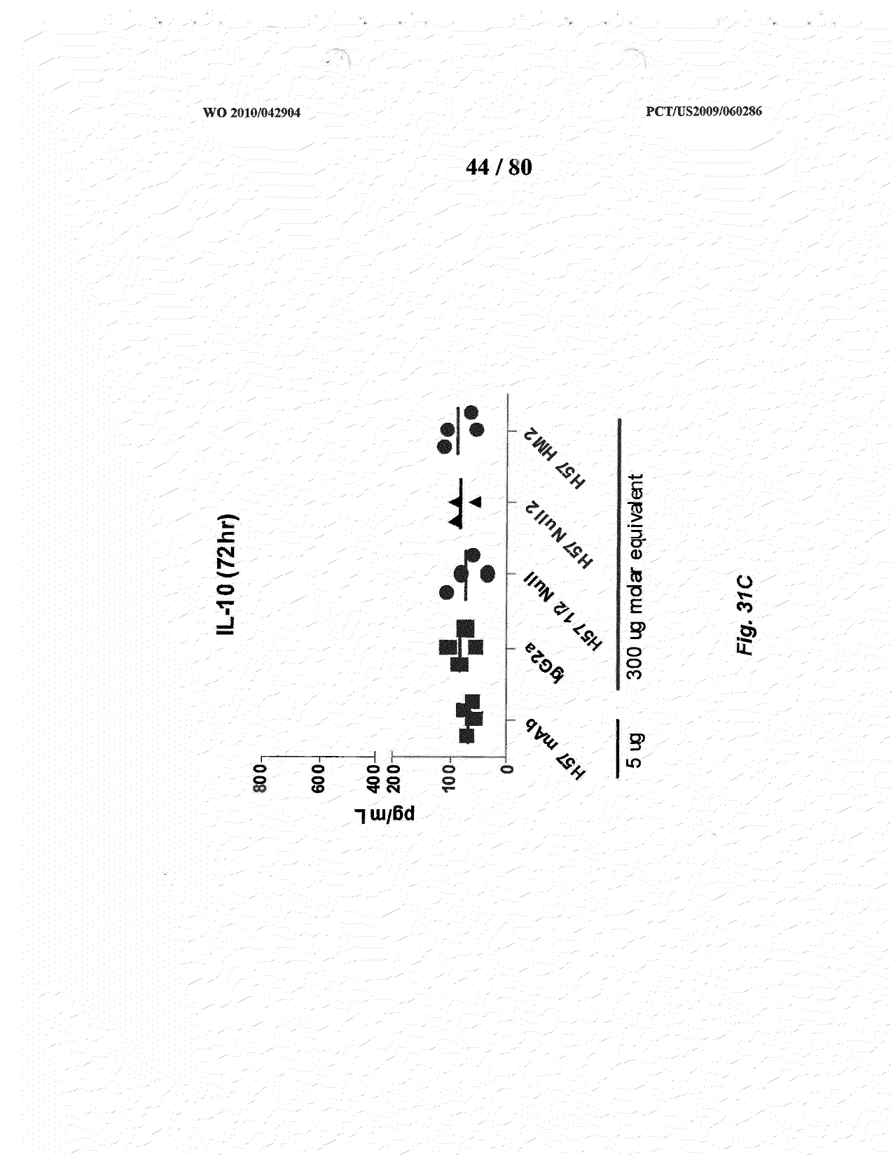

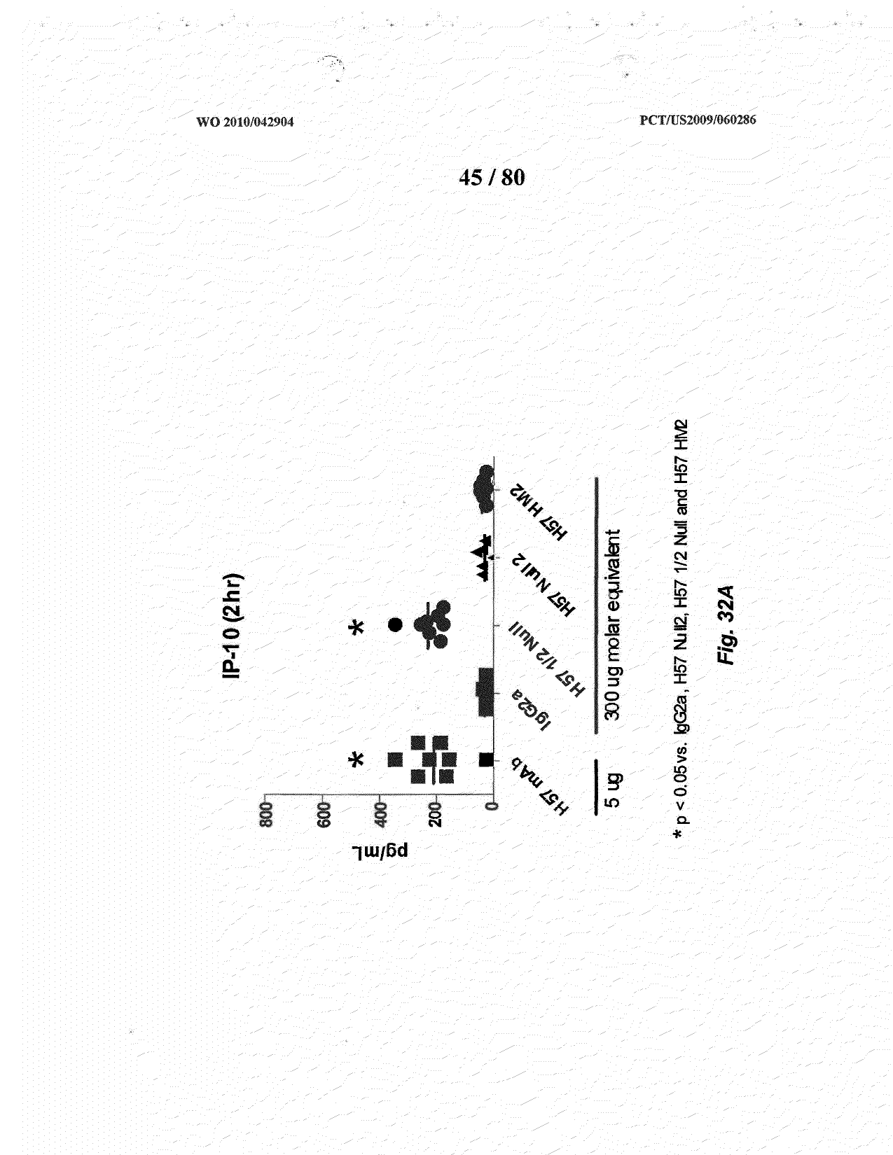

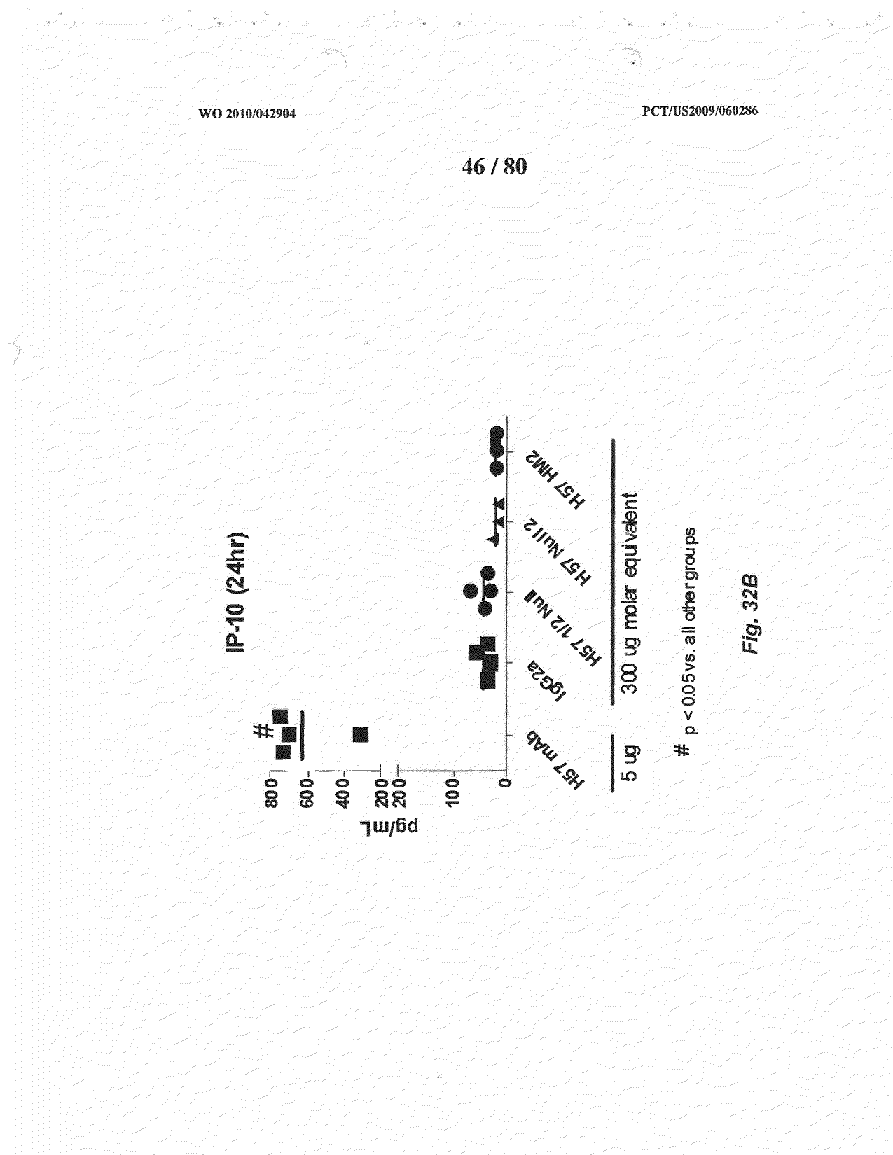

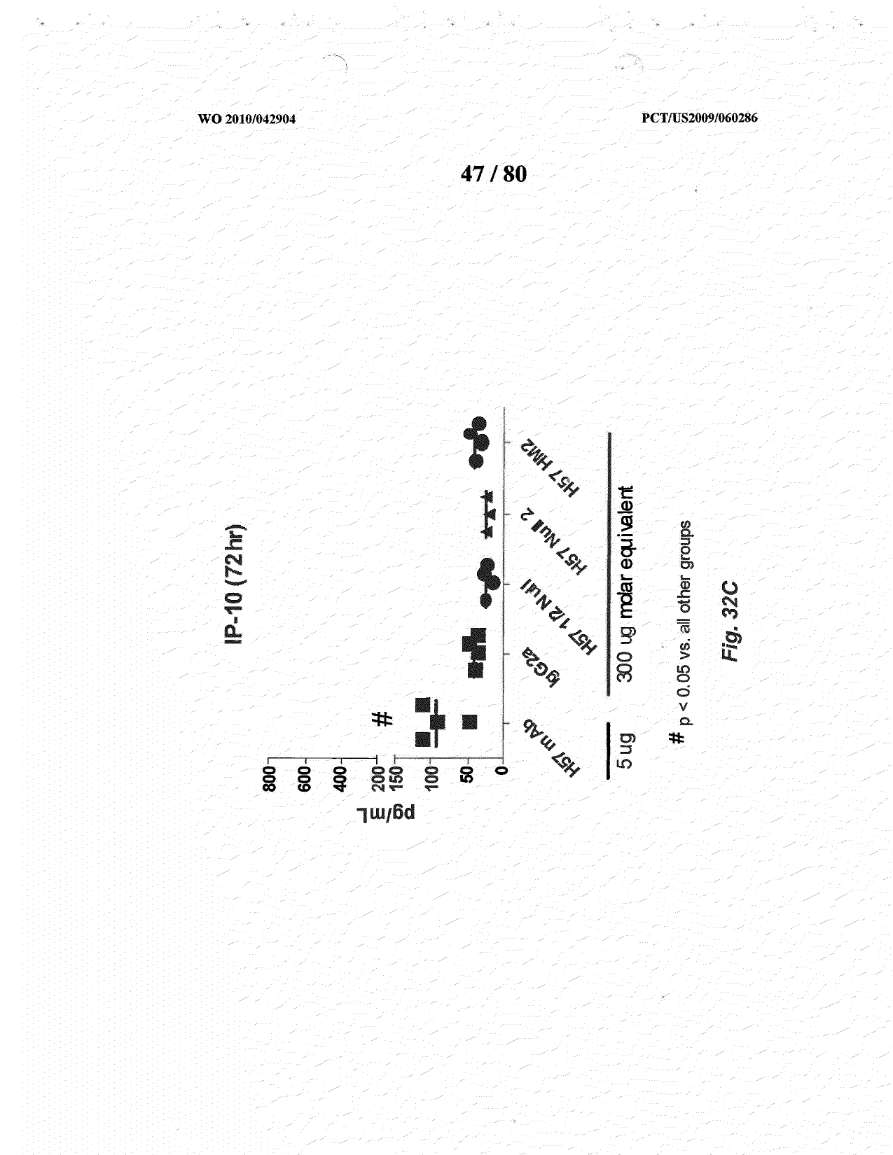

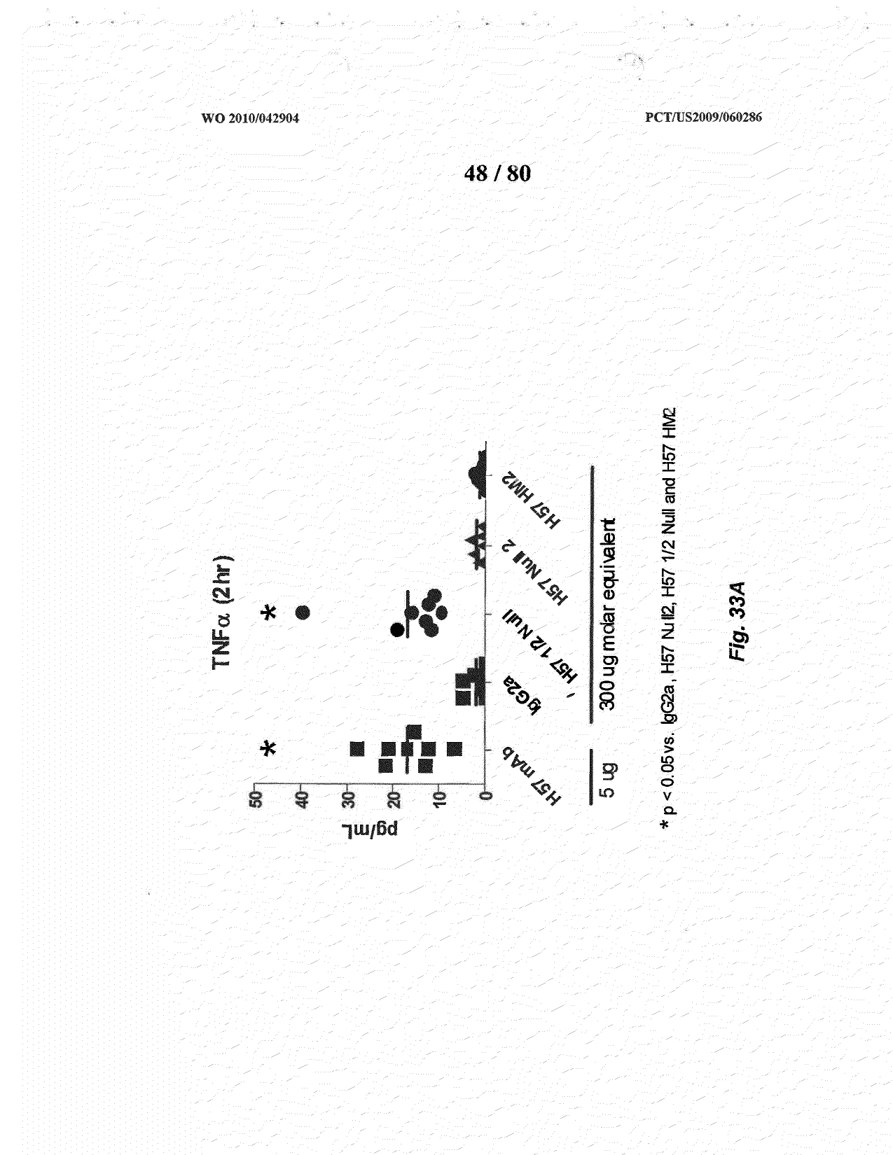

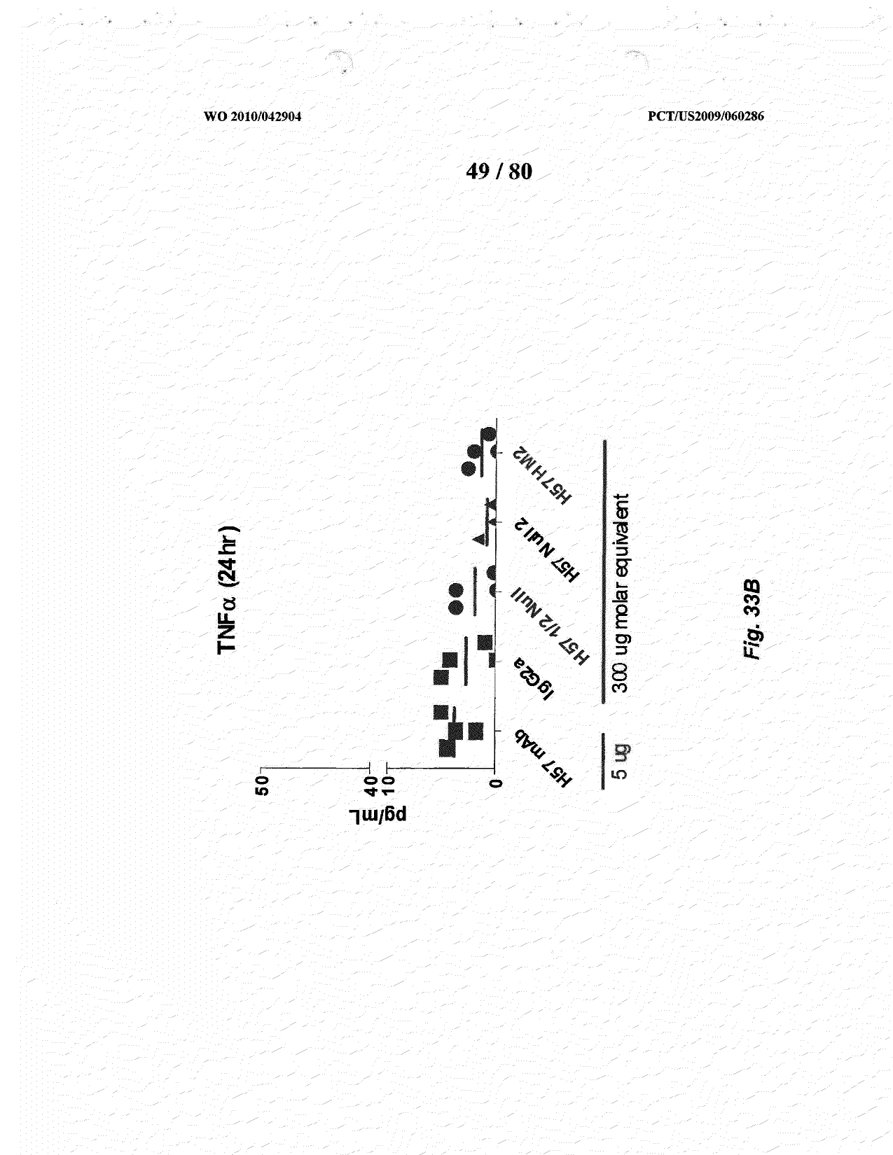

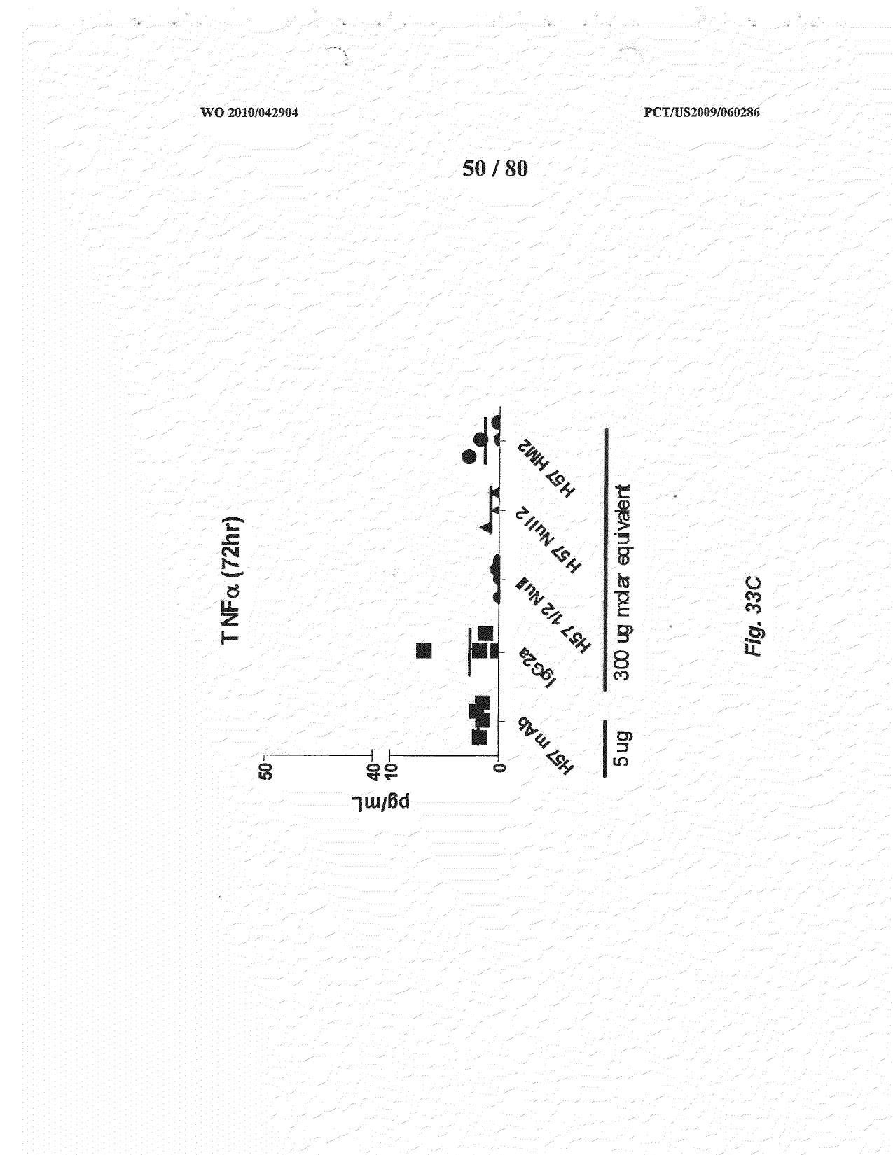

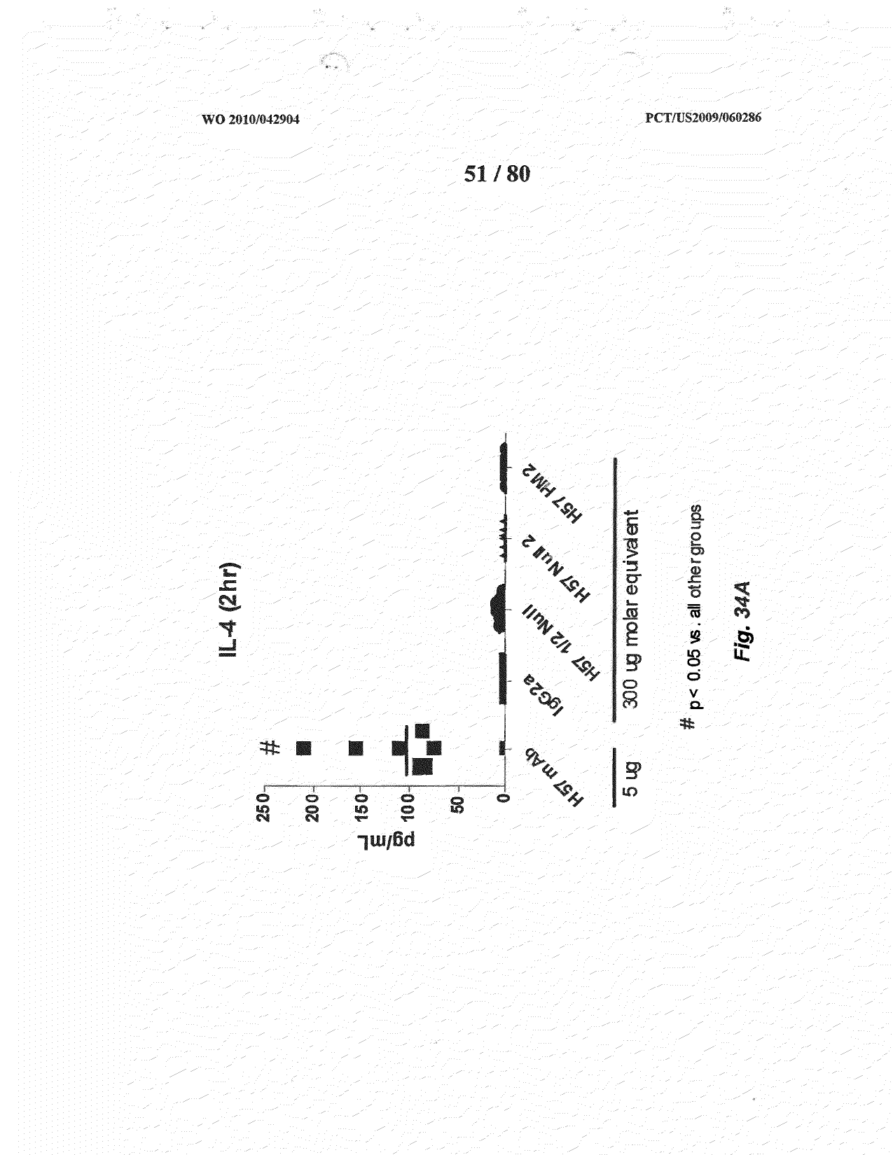

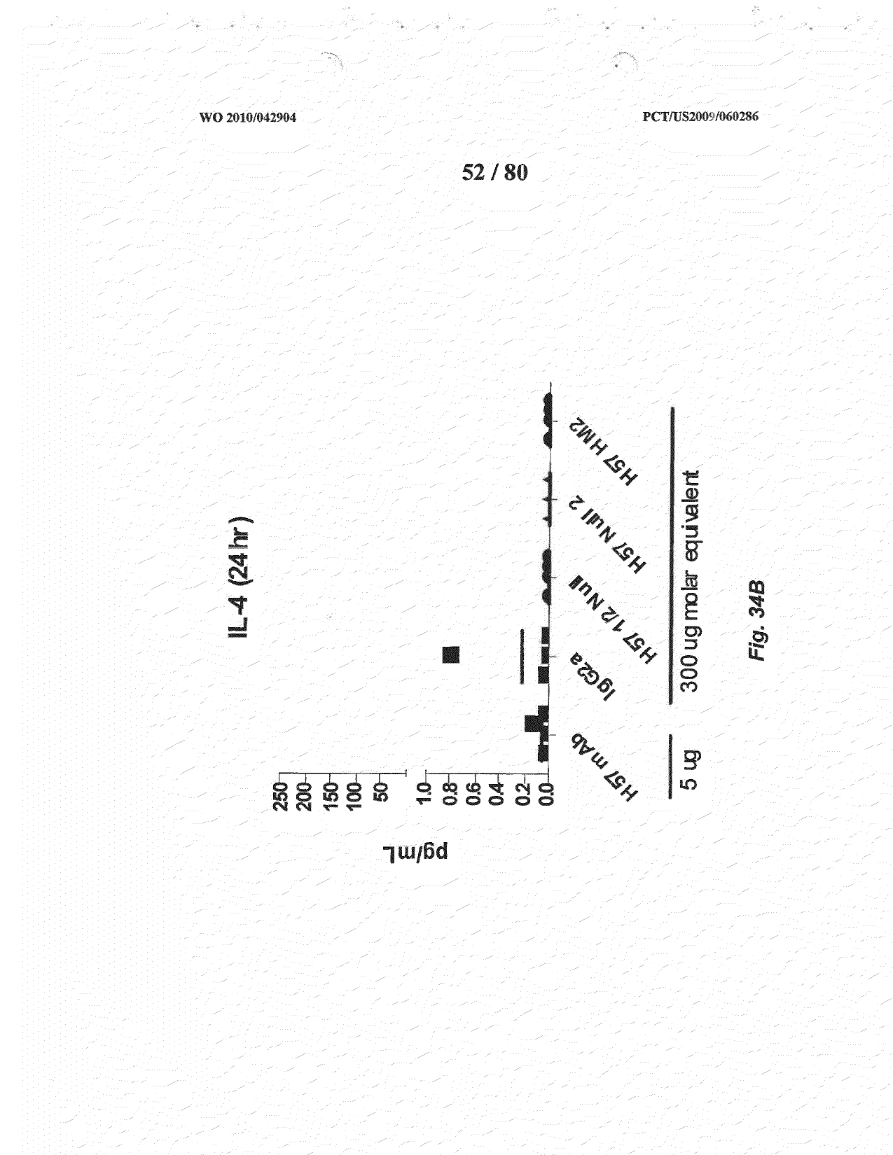

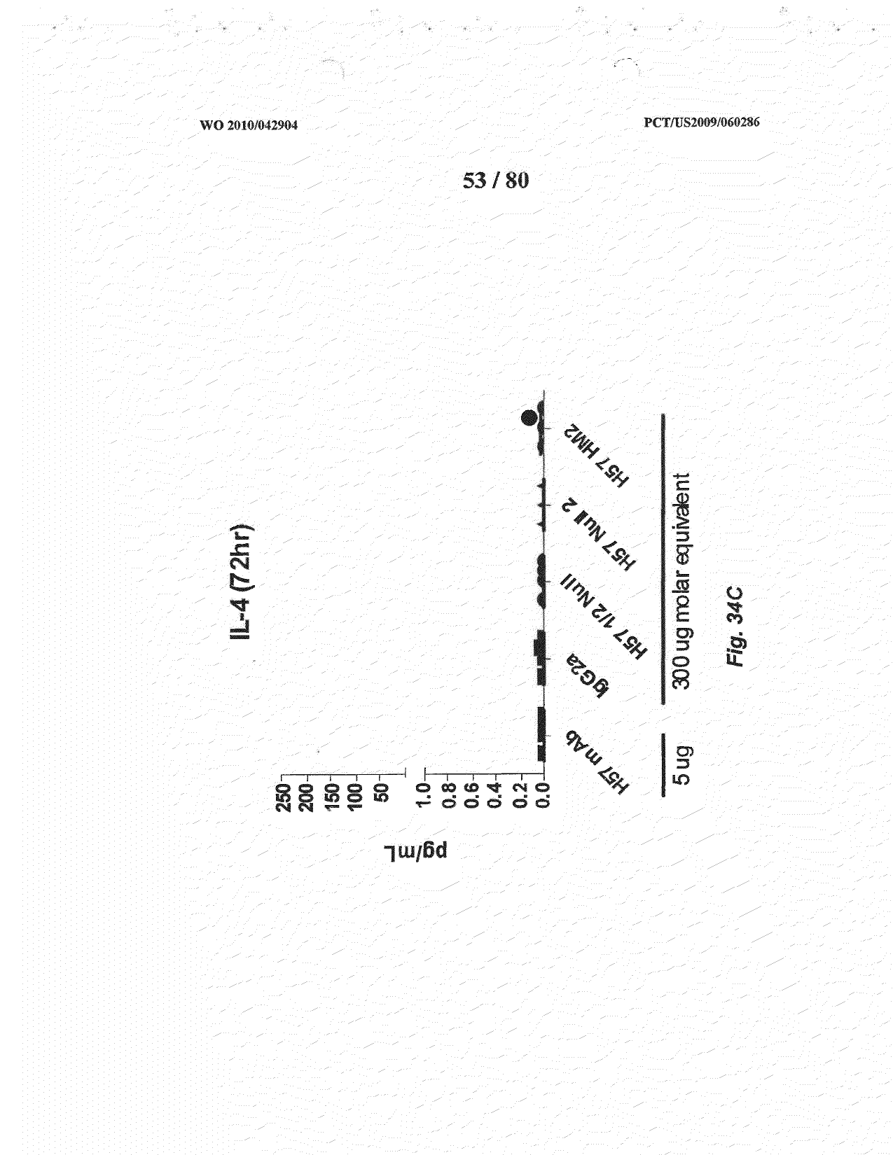

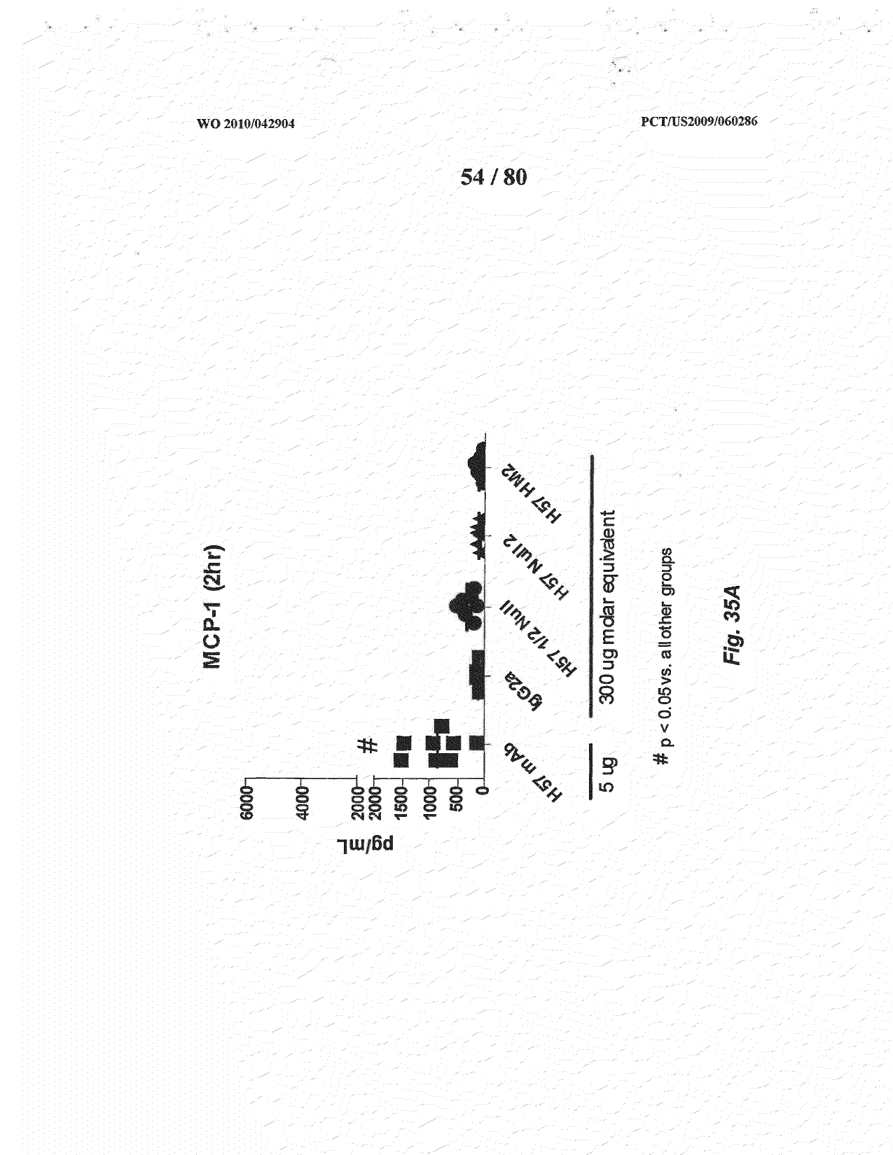

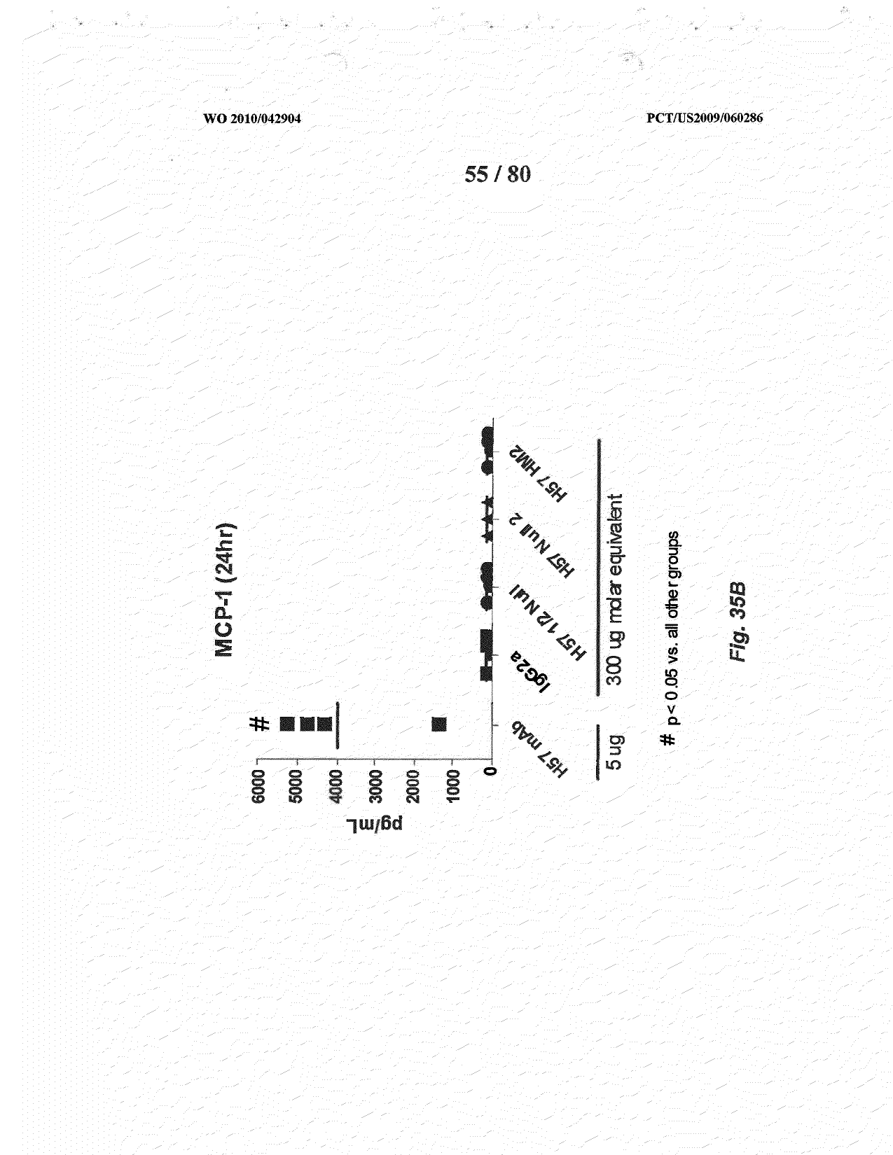

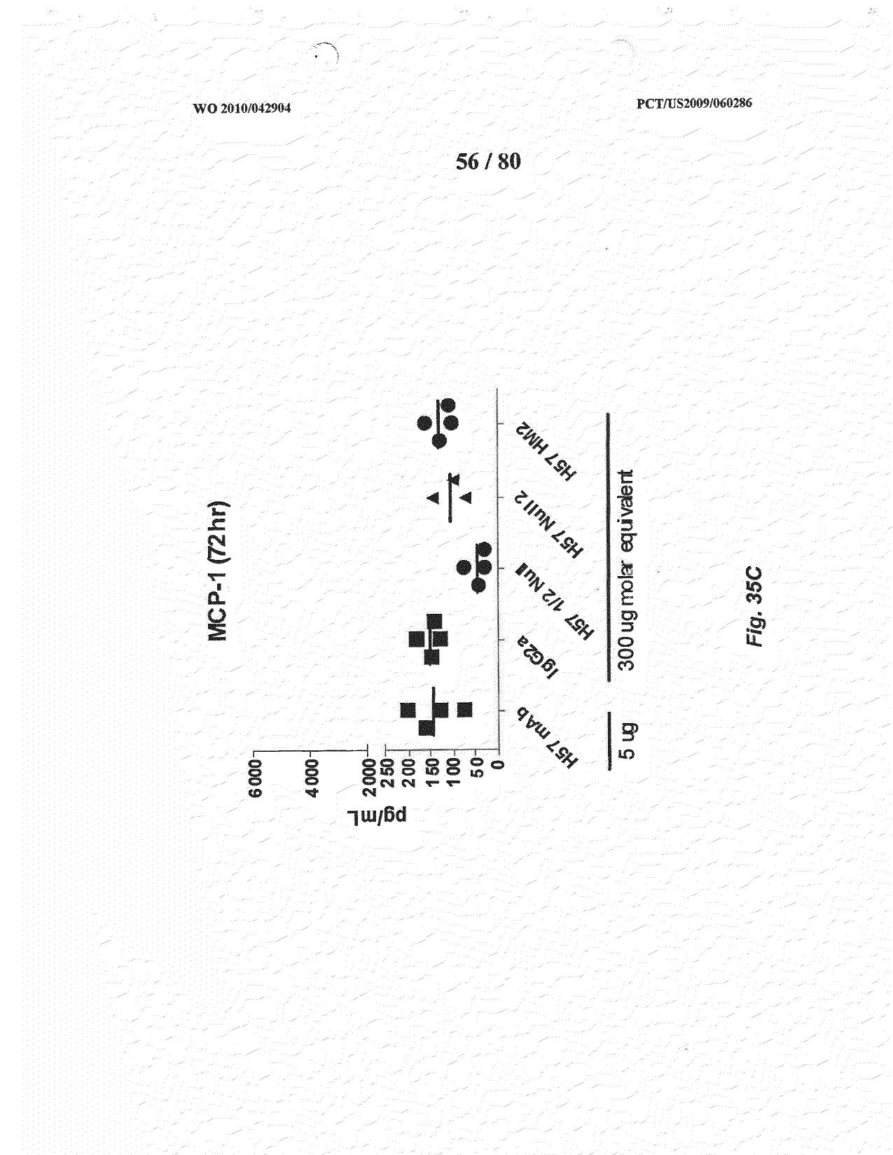

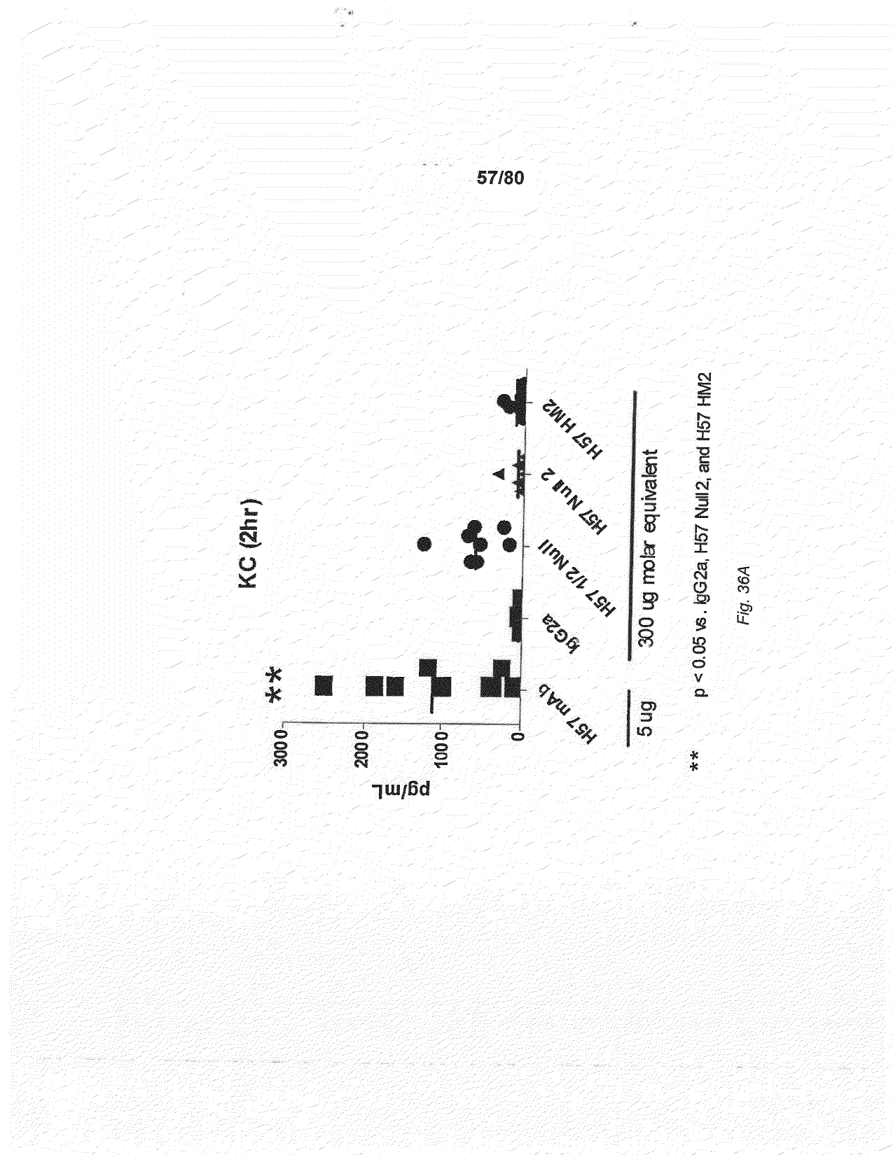

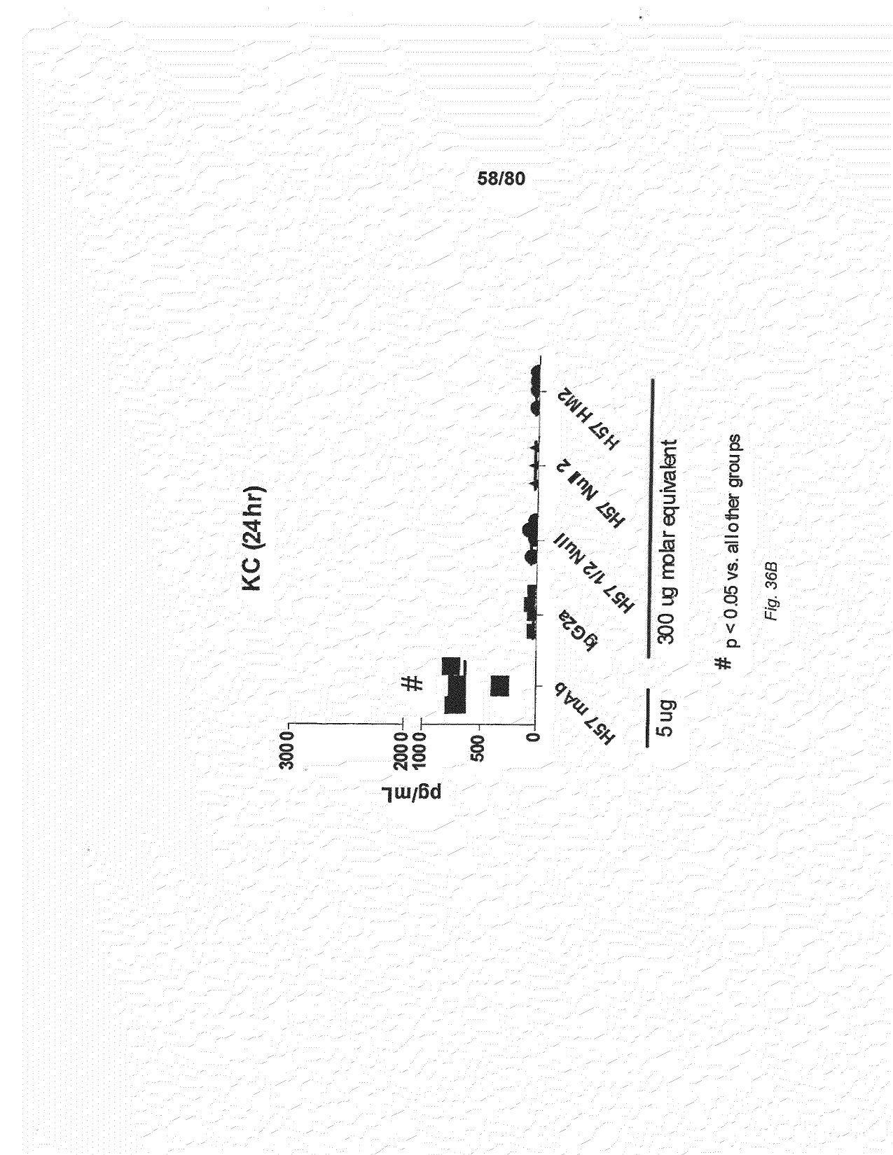

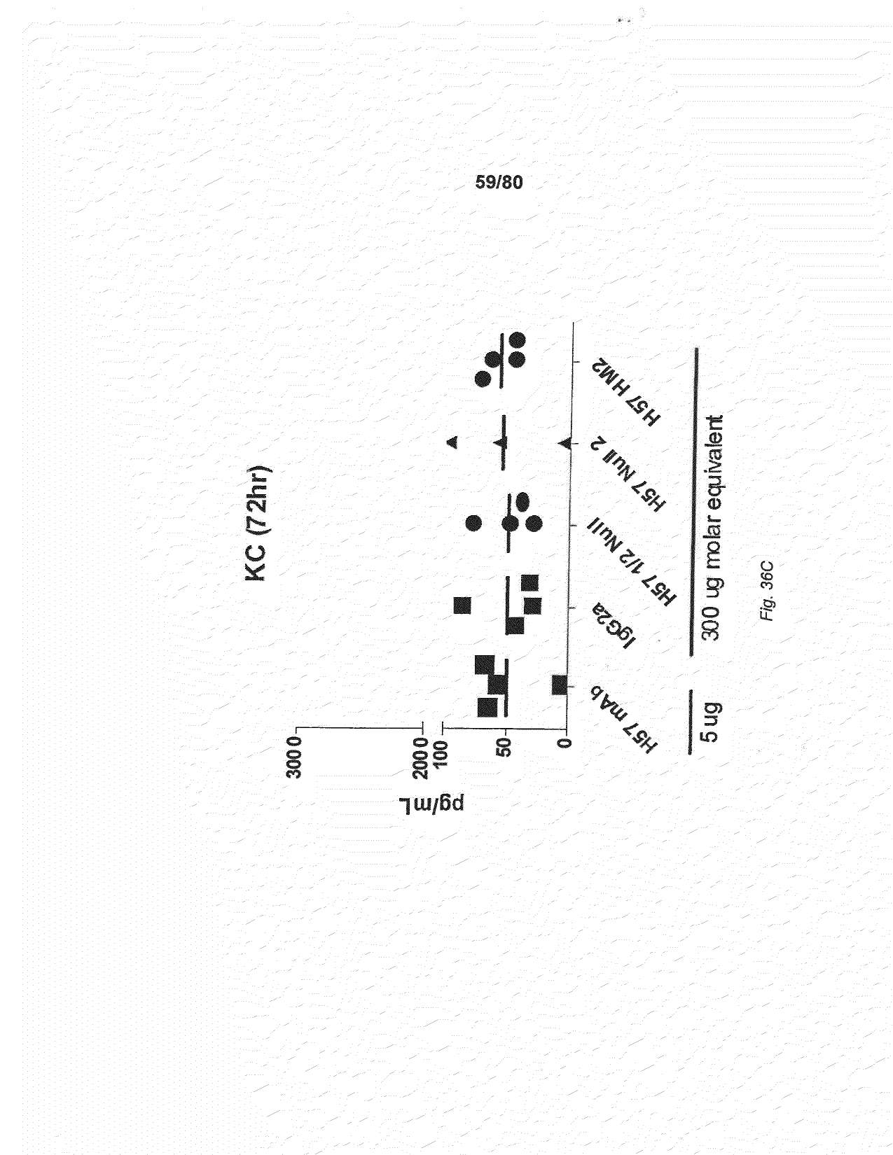

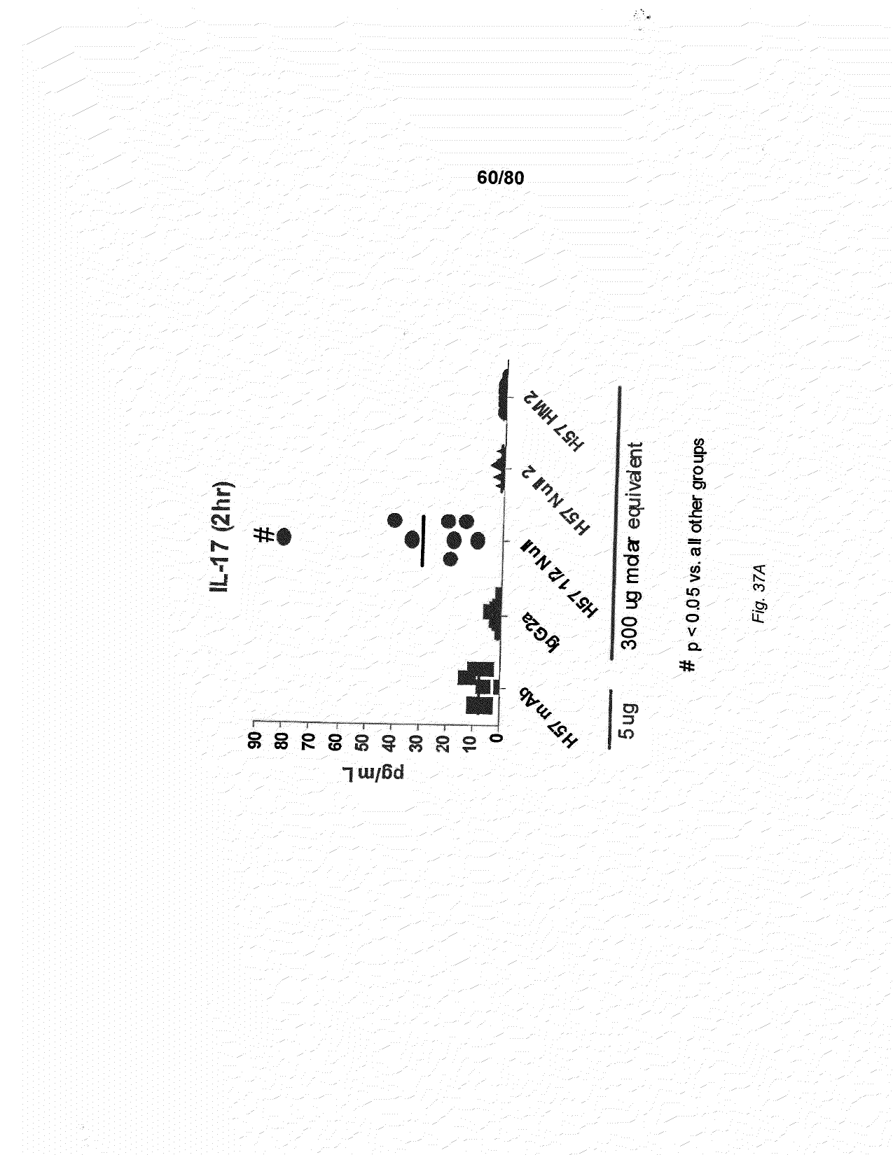

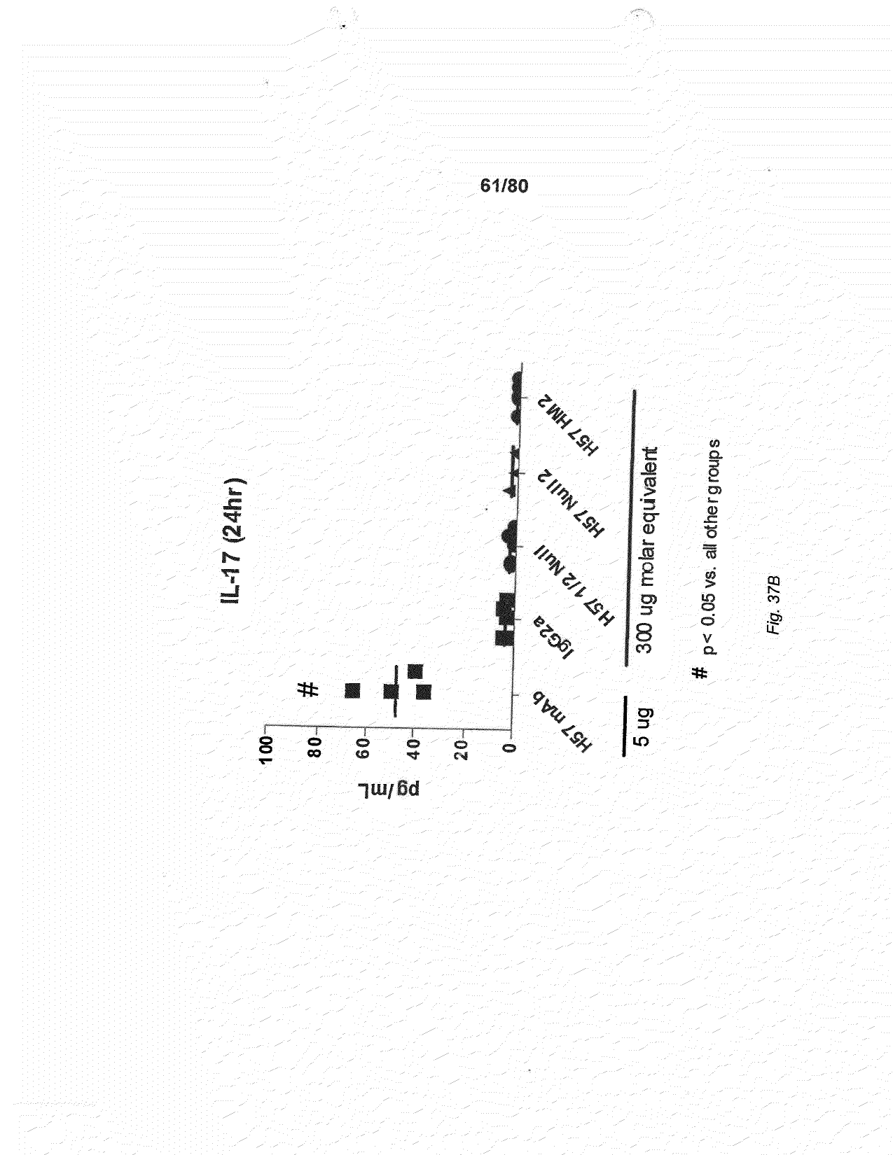

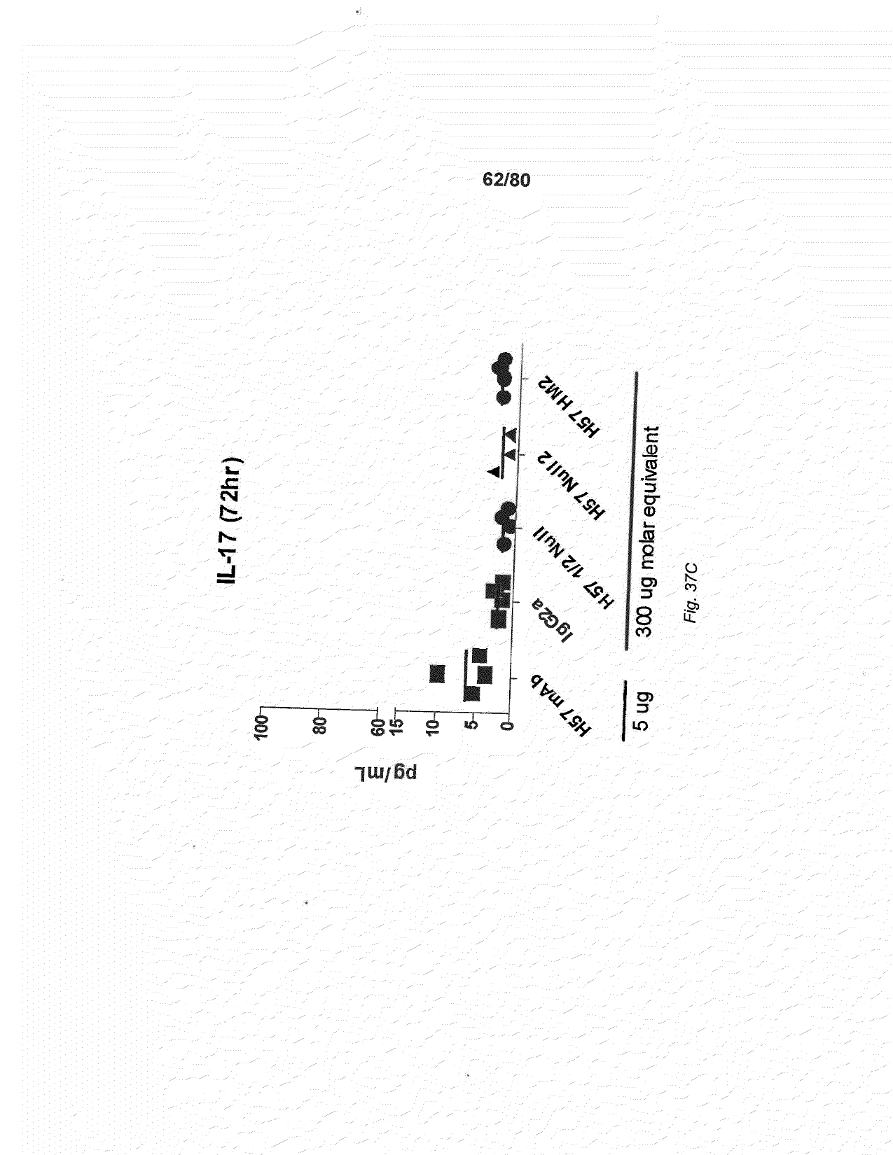

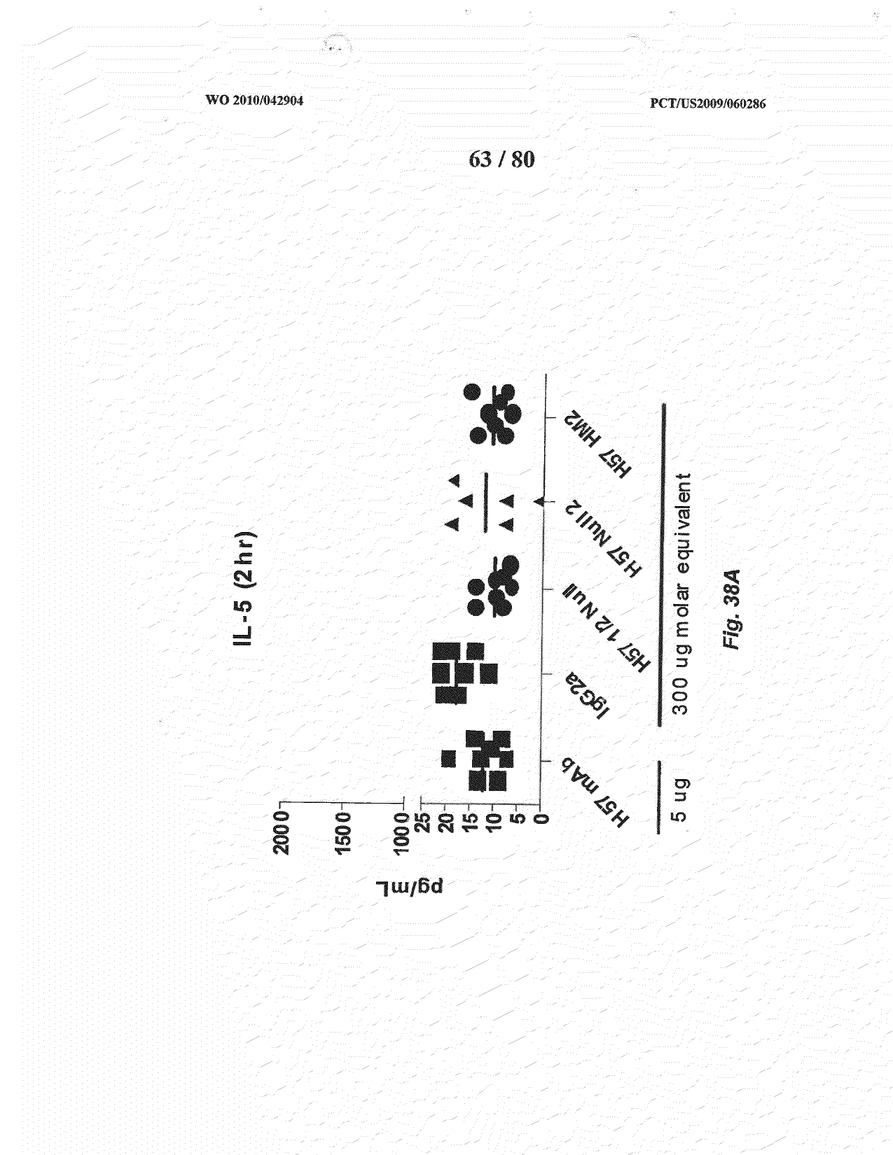

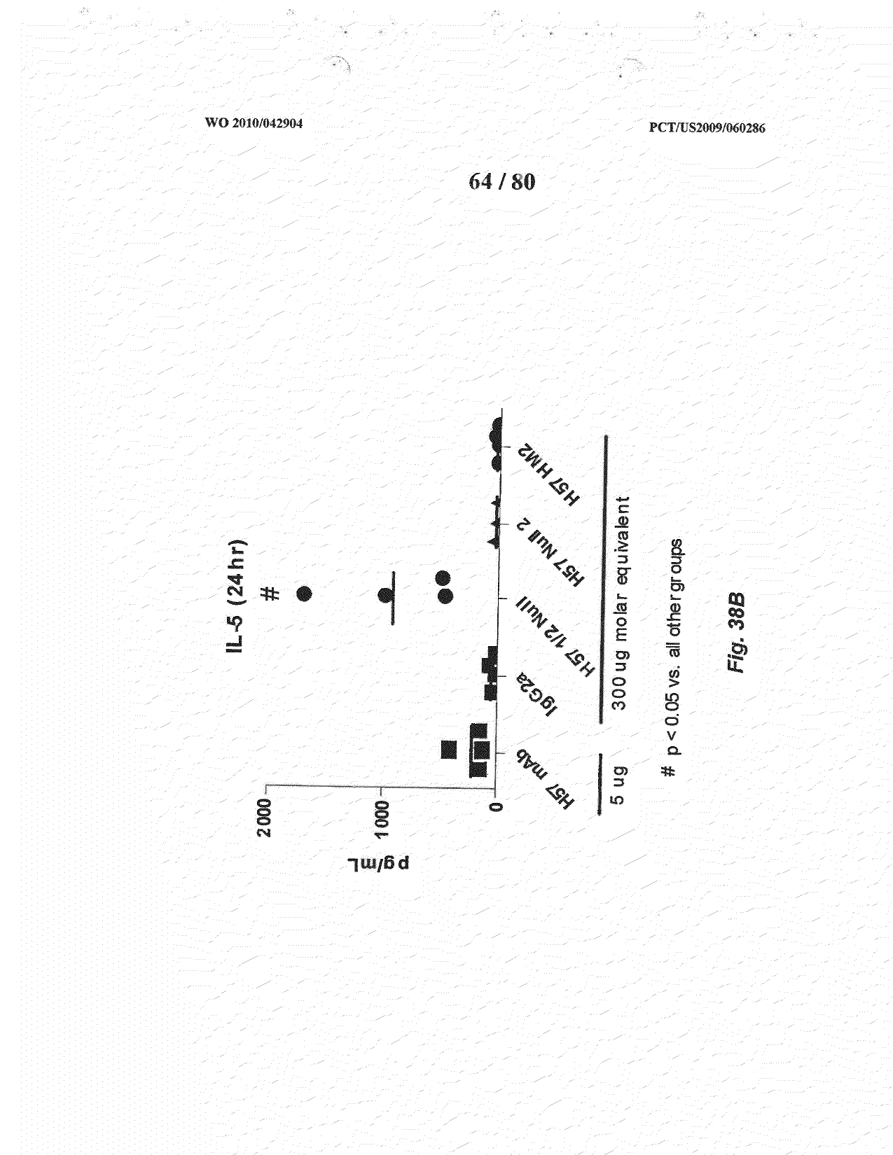

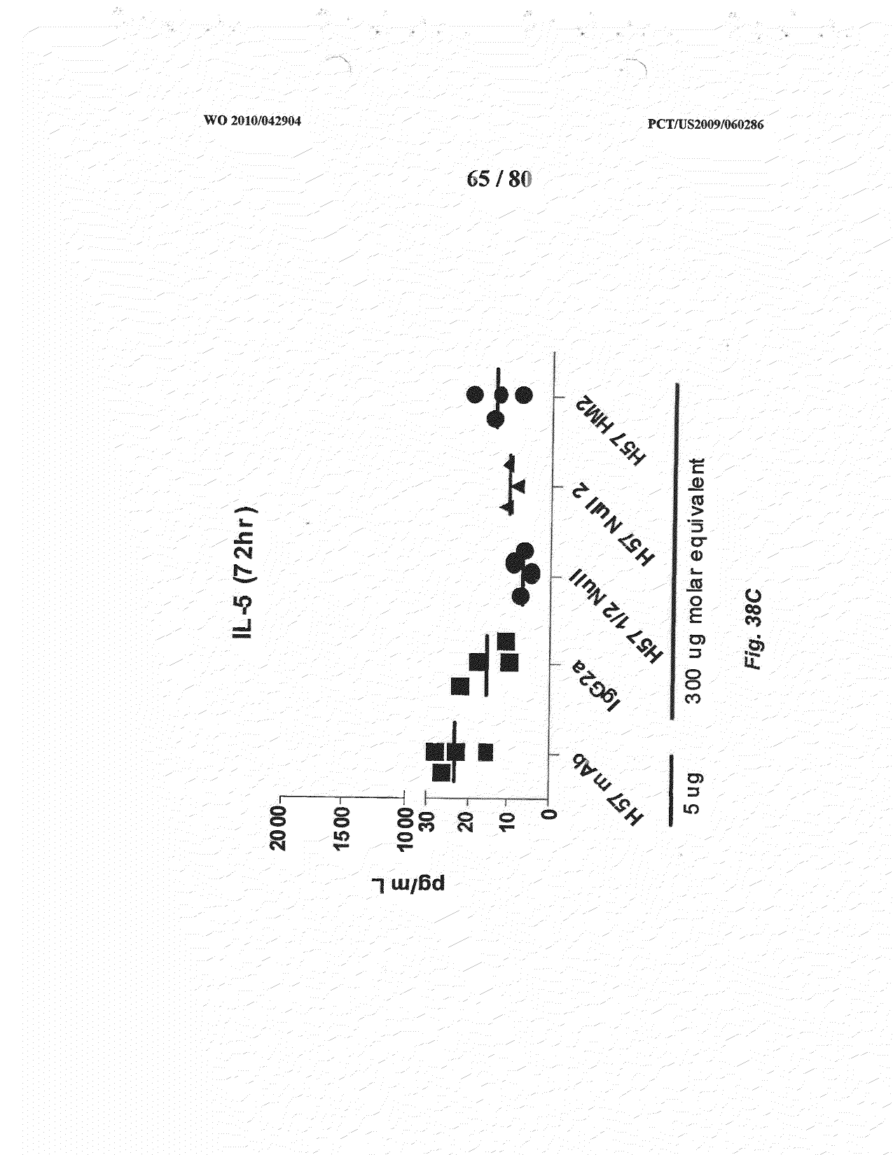

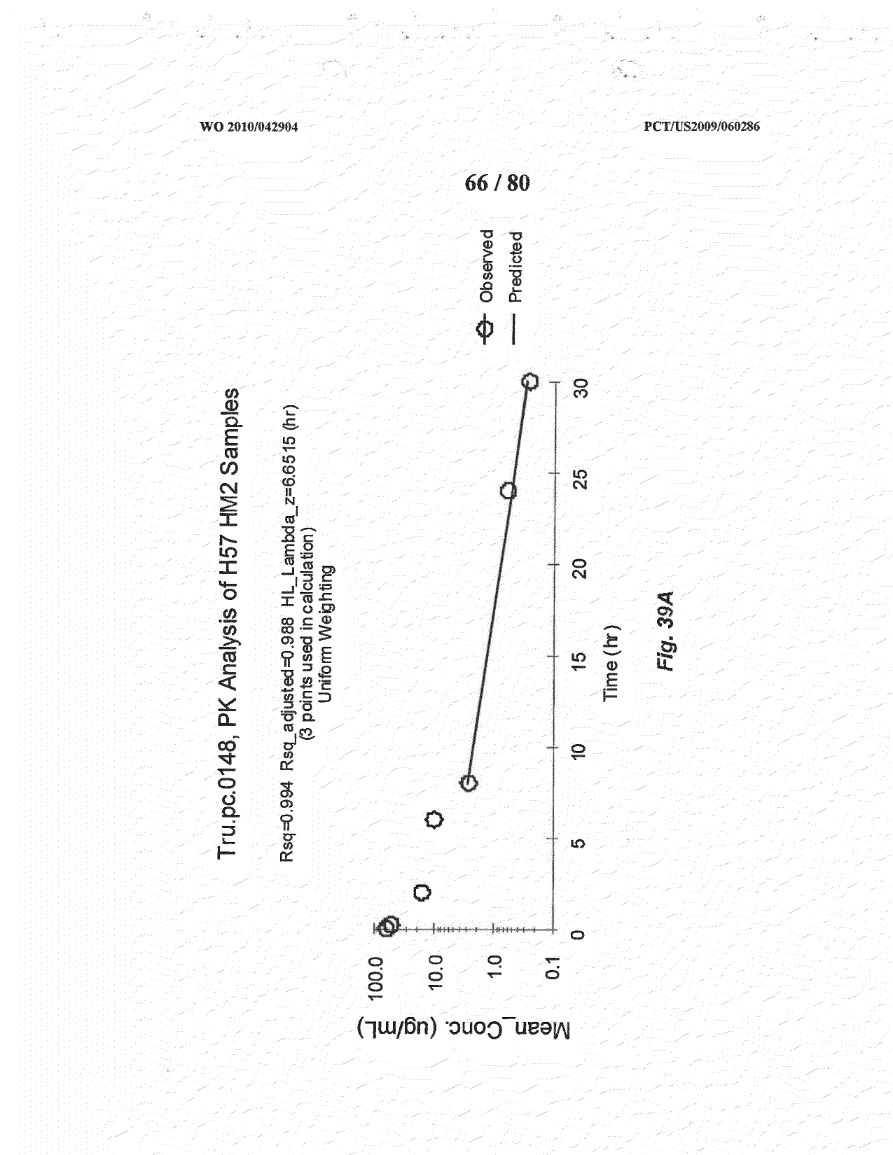

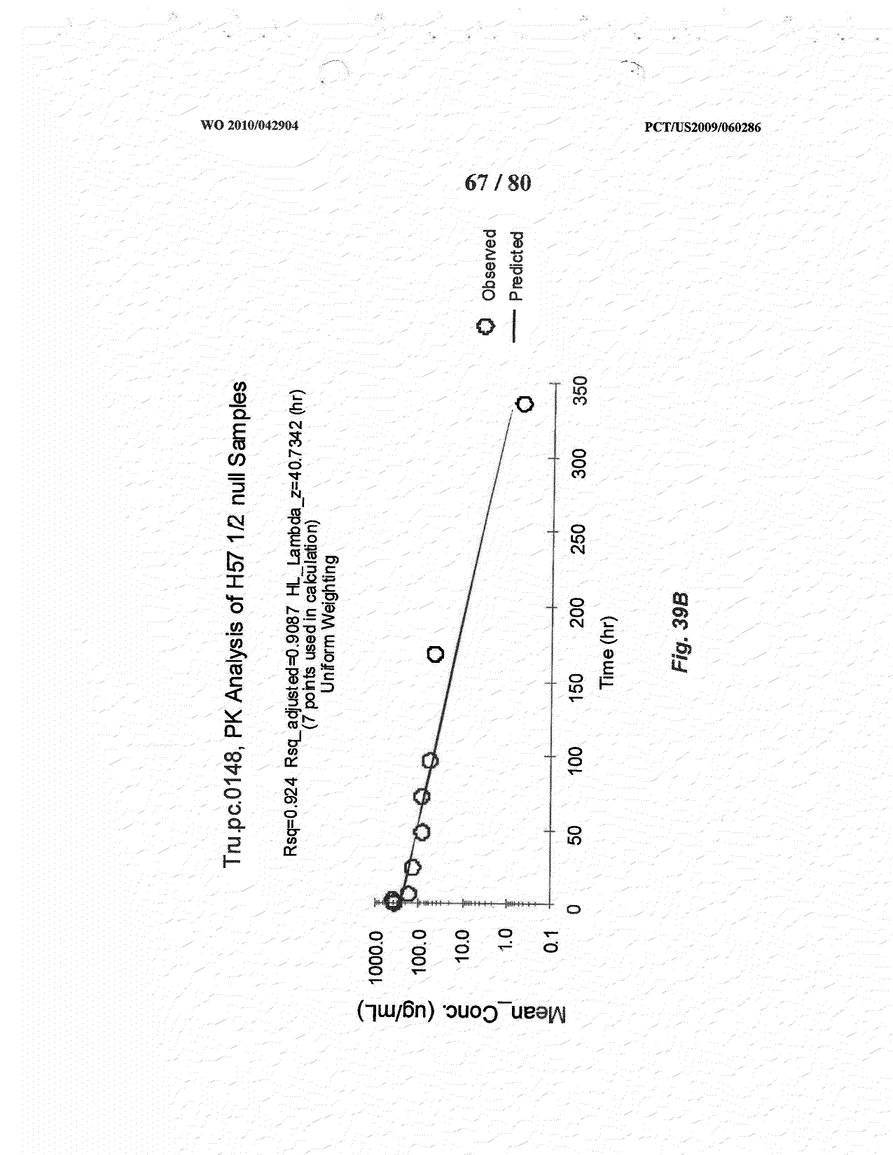

FIGS. 30A-38C show that (1) H57 Null2 and H57 HM2 did not cause increase in cytokine production compared to IgG2a, and (2) H57 half null treatment elevated the levels of IL-2, IL-10, IP-10, TNFα, and IL-17 at 2 hours post injection, but the levels of all but IL-5 returned to normal levels by 24 hours post injection.

### Example 13

### Pharmacokinetic Study of Exemplary Fusion Proteins Containing H57Binding Domain

Female BALB/c mice were injected intravenously (IV) at time 0 with 200 μL of PBS containing 200 μg of H57 Null2 (SEQ ID NO:96), H57-HM2 (SEQ ID NO:306) or H57 half null SMIP protein (SEQ ID NO:304). Three mice per group were injected for each time point: For H57-HM2 SMIP protein, serum samples were obtained at 15 min and 2, 6, 8, 24, 30, 48, 72, 168, and 336 hr, and for H57 Null2 and H57 half null, additional time points were taken at 96 and 504 hr, but the 8 and 30 hr samples were omitted. Anesthetized mice were exsanguinated via the brachial plexus or cardiac puncture at the indicted time points after injection, and serum was collected as described below.

Serum concentrations of BC3 IgG4-AA-N297A and BC3 IgG2-AA-N297A were determined with a sandwich ELISA using a goat anti-human IgG Fc specific antibody as the capture reagent, and HRP conjugates of antibodies to human IgG4 or IgG2 to detect bound BC3 IgG4-AA-N297A or BC3-IgG2-AA-N297A SMIP, respectively. Serum concentrations for OKT3IgG4-AA-N297A and BC3-HM1 were determined in a FACS-based binding assay using the CD3′ Jurkat cell line. Jurkat cells were incubated in 96 well flat bottom plates along with serum samples from mice injected with OKT3 IgG4-AA-N297A or BC3-HM1. Each serum sample was tested in triplicate at one dilution. The dilutions used for samples varied for different time points, but ranged from 1:20 to 1:15,000 for OKT3 IgG4-AA-N297A and 1:20 to 1:1000 for BC3-HM1. (Pooled samples from mice injected with OKT3 IgG2-AA-N297A or BC3-HM1 were tested in a preliminary assay, so the appropriate dilution for each sample was known.) Cells were incubated for an hour in the presence of the diluted serum samples or standards (see below) and were washed before the addition of the detection reagent. Binding of OKT3 Ig4-AA-N297A to Jurkat cells was detected using a PE-conjugated goat anti-human IgG Fc [symbol] fragment-specific antibody, whereas binding of BC3-HM1 to Jurkat cells was detected using a PE-conjugated anti-His antibody. Serum concentrations for H57 Null2, H57-HM2, and H57 half null were determined in a FACS-based binding assay using EL4 cells, a mouse T cell line. EL4 cells were blocked with anti-mouse CD16/CD32, and then incubated in 96-well flat bottom plates along with serum samples from mice injected with H57-null2. Each serum sample was tested in triplicate at one dilution. The dilutions used for samples varied for different time points, but ranged from 1:500 to 1:10,000. (Pooled samples from mice injected with H57-null2 were tested in a preliminary assay, so the appropriate dilution for each sample was known.) Standard curves consisted of various known concentrations of H57 Null2 spiked into FACS buffer, run in triplicate. Serum was not added to standard curves because development work showed that serum at dilutions greater than 1:50 had no effect on standard curves, and much larger dilutions (minimum of 1:500) of serum were required for PK samples.

EL4 cells were incubated for an hour in the presence of the diluted serum samples or standards and were washed before the addition of the detection reagent. Binding of H57 Null2 and H57 half null to EL4 cells was detected using a PE-conjugated donkey anti-mouse IgG (H+L) antibody, whereas binding of H57-HM2 to EL4 cells was detected using a PE-conjugated anti-His antibody. The samples were analyzed by flow cytometry. The mean fluorescence intensities (MFI) were imported into Softmax Pro software to calculate serum concentrations and to determine precision and accuracy of standard curves.

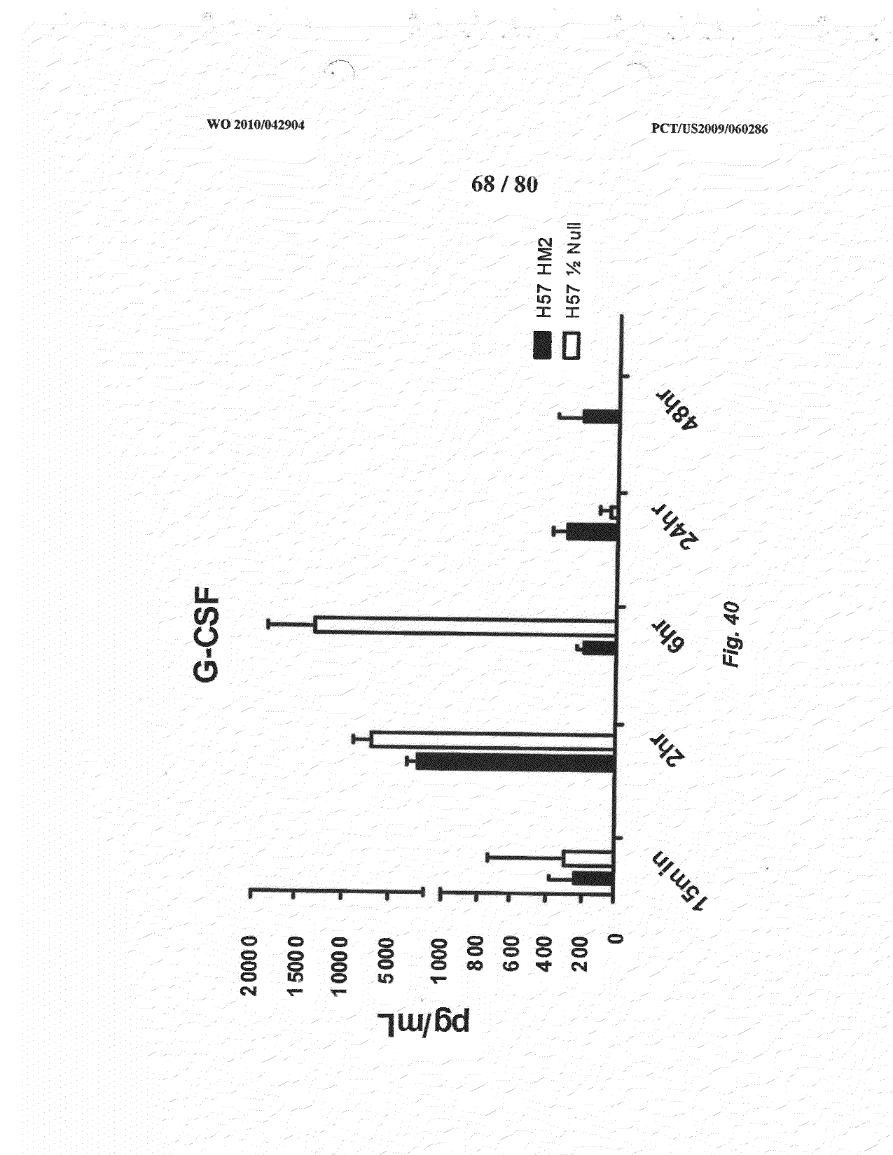

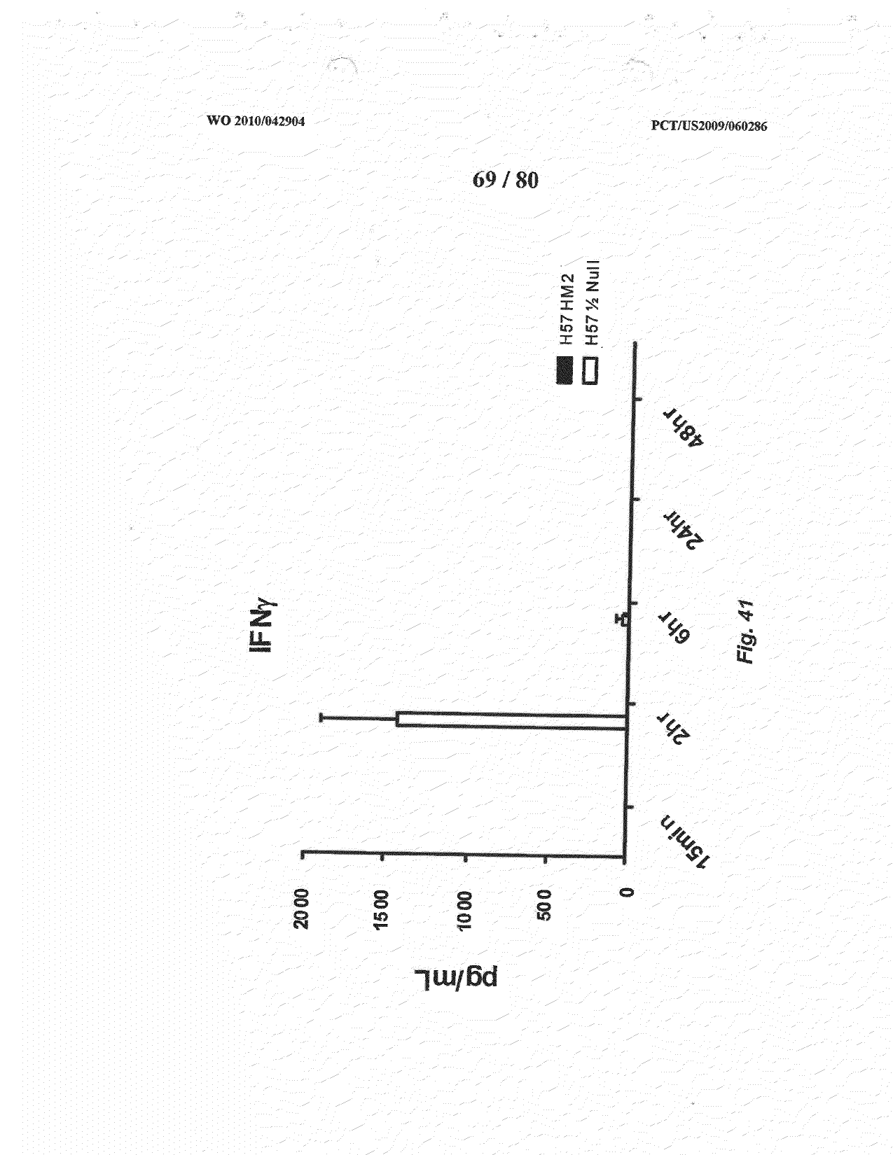

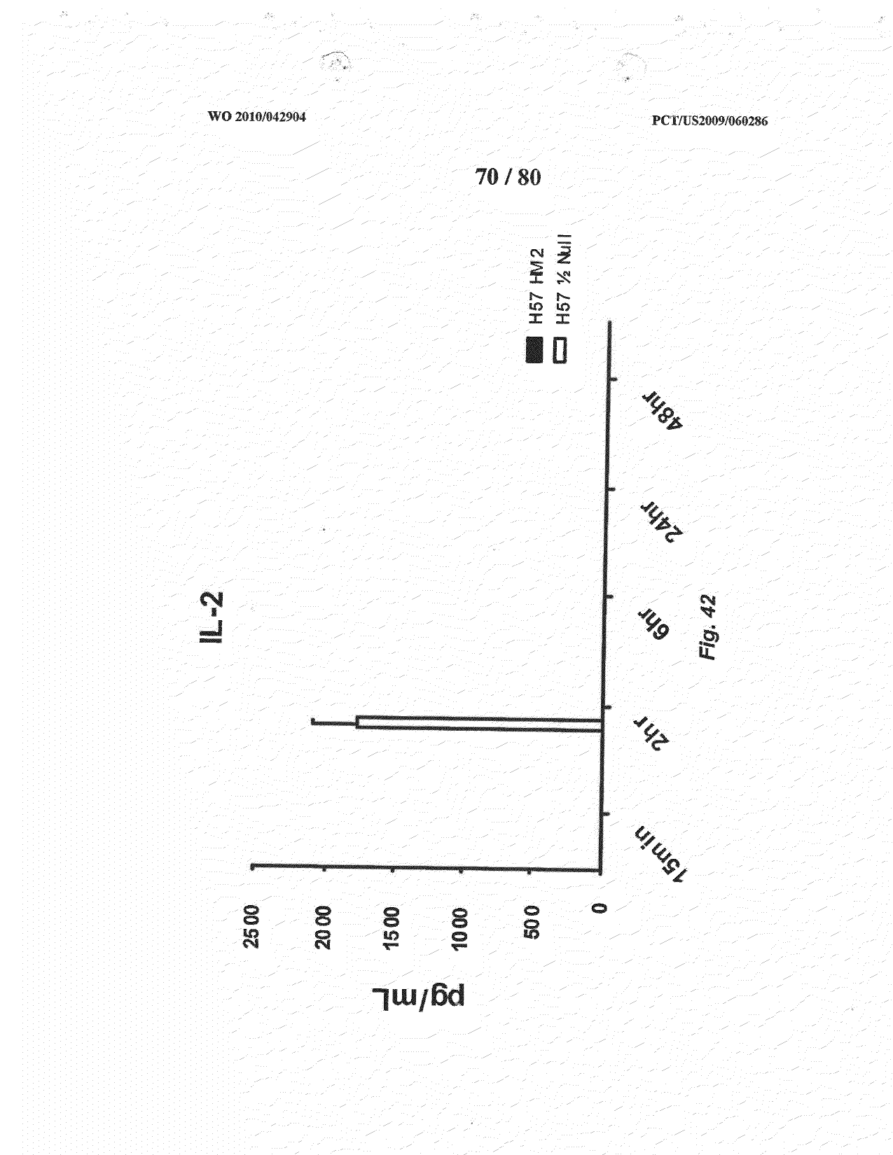

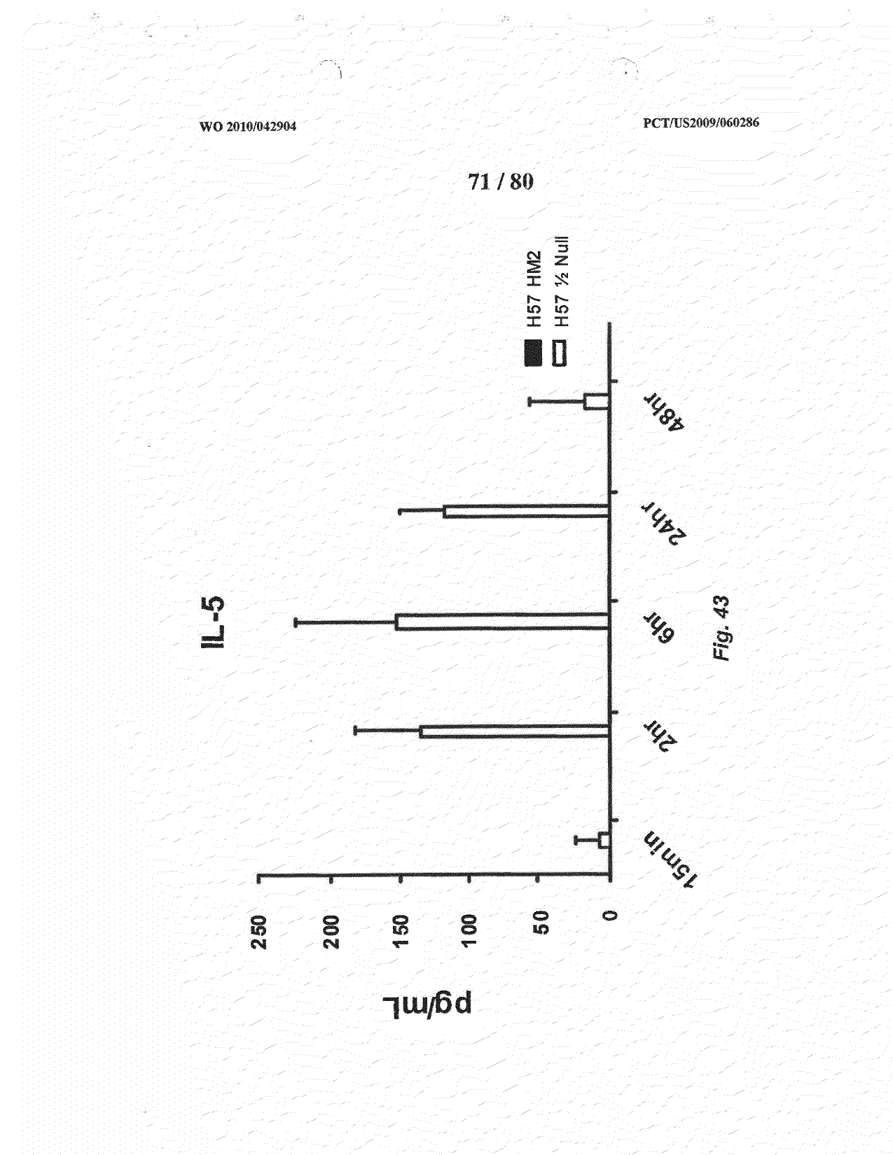

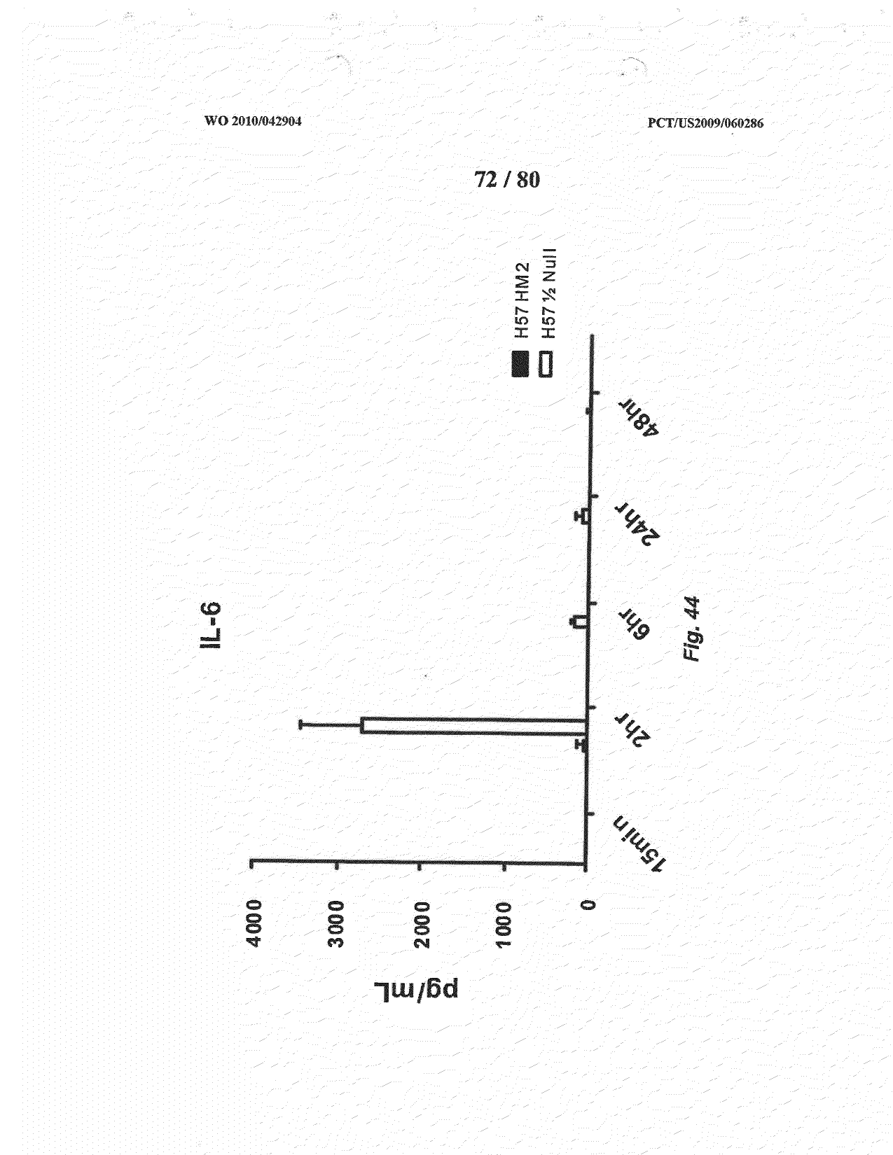

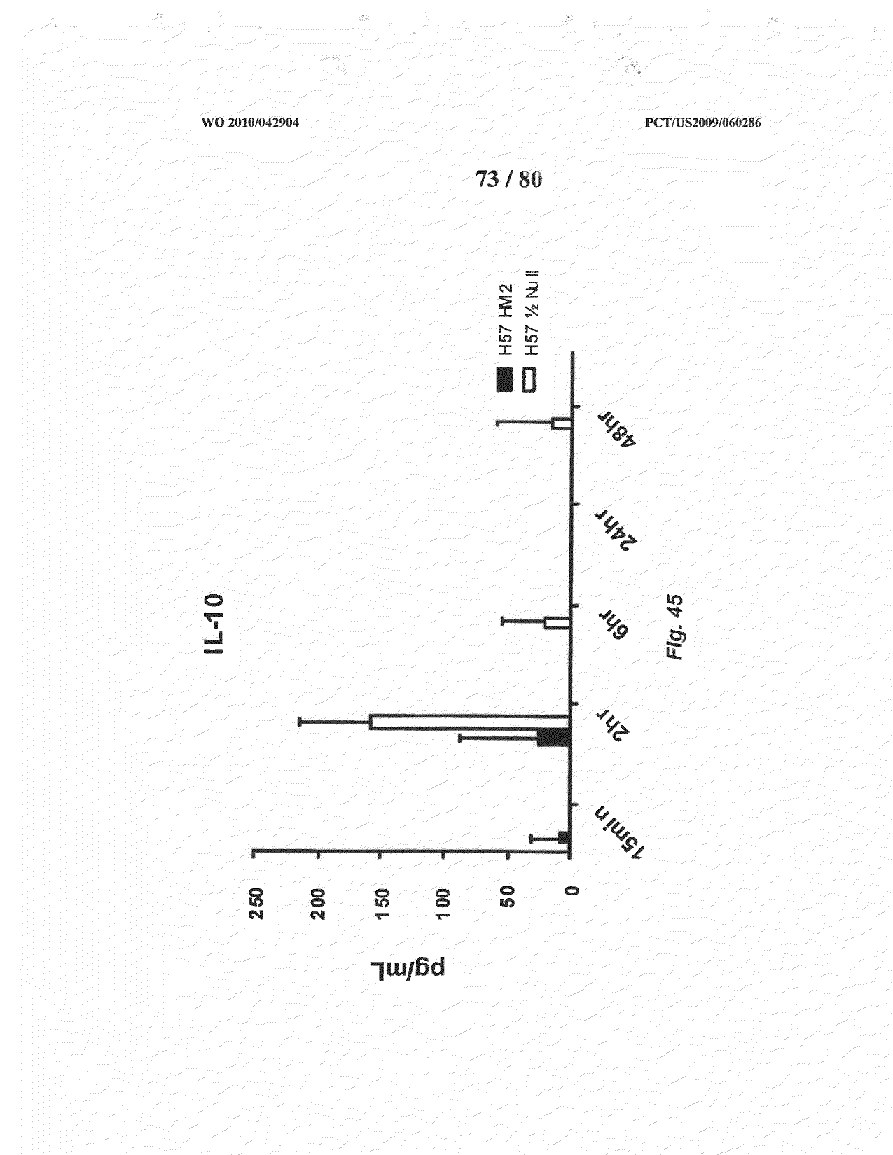

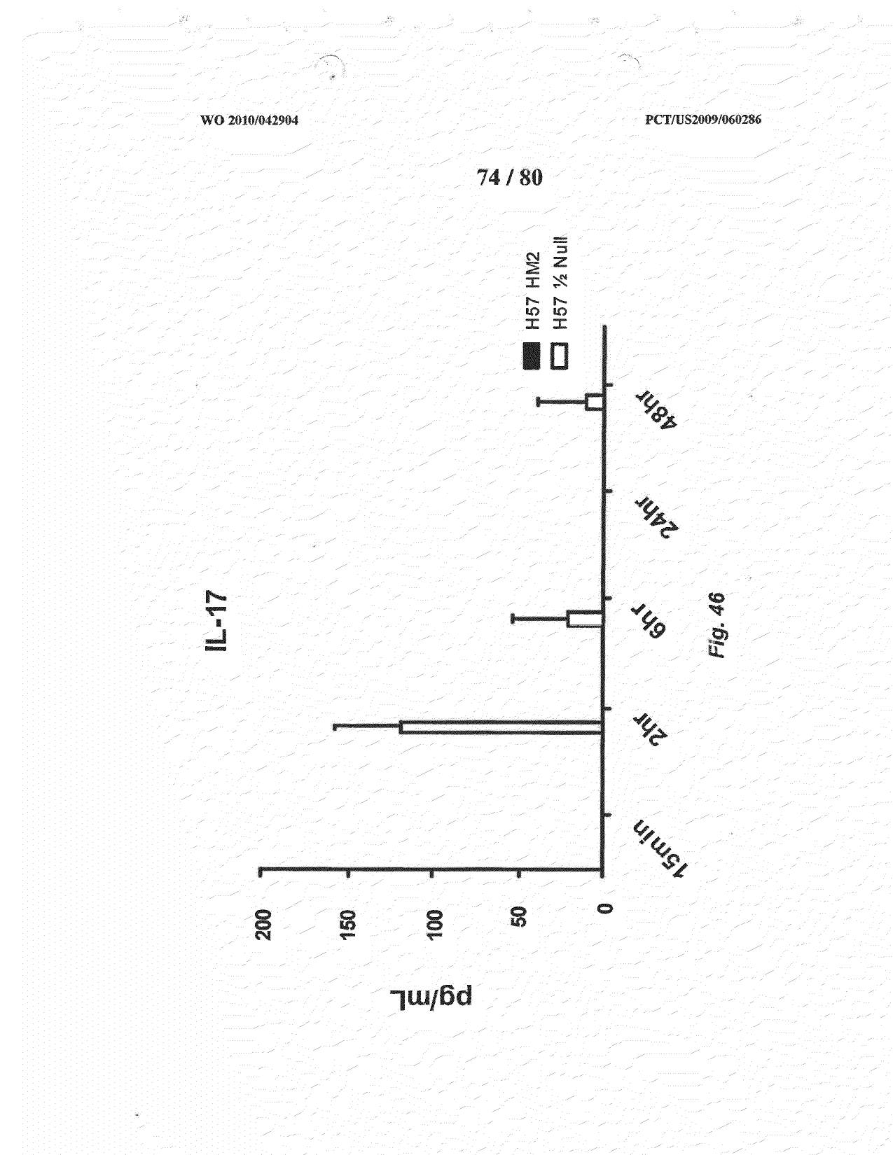

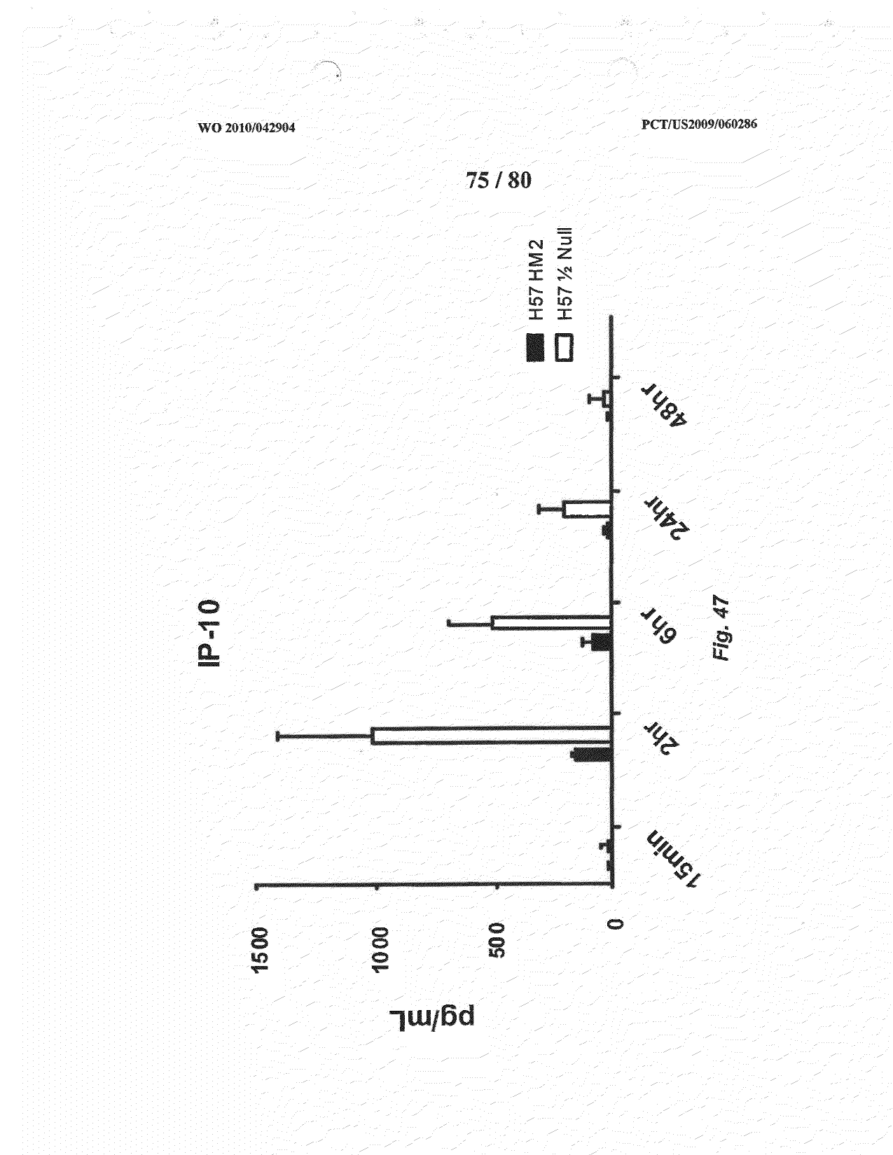

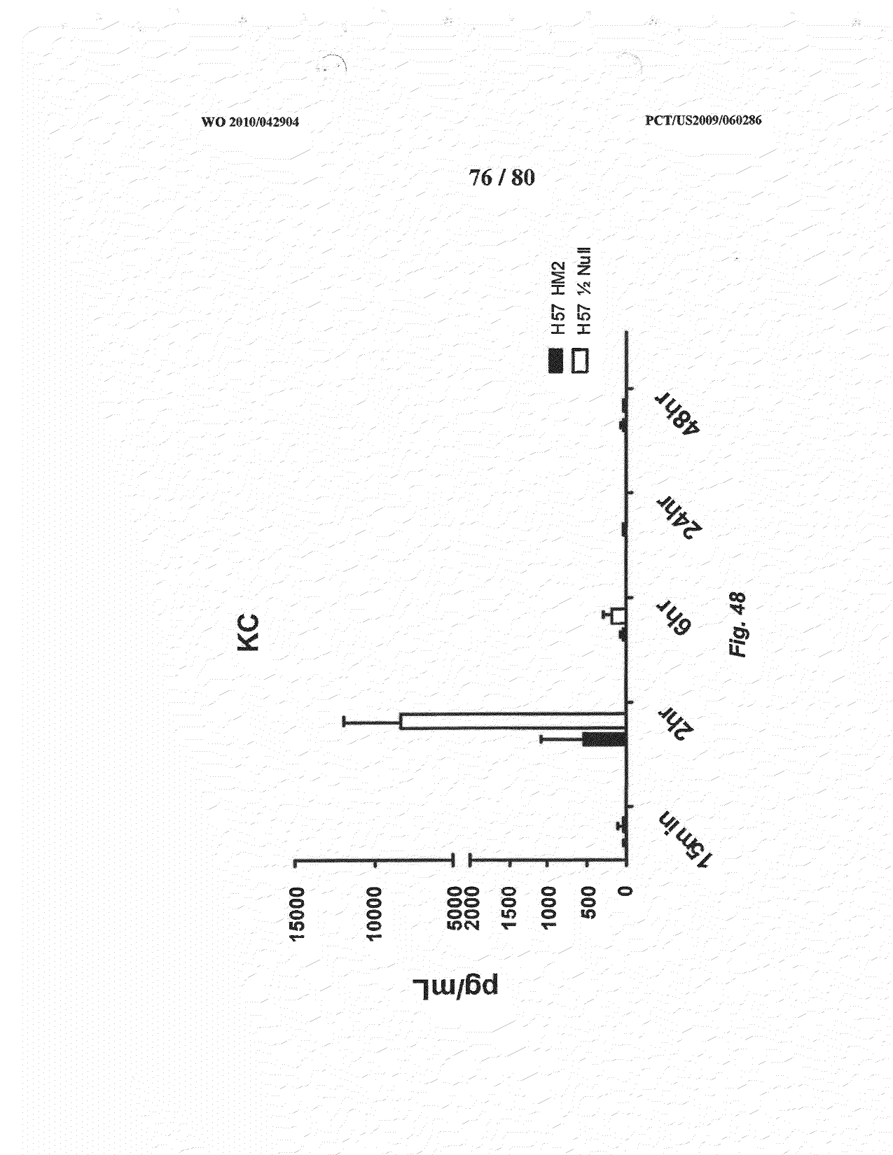

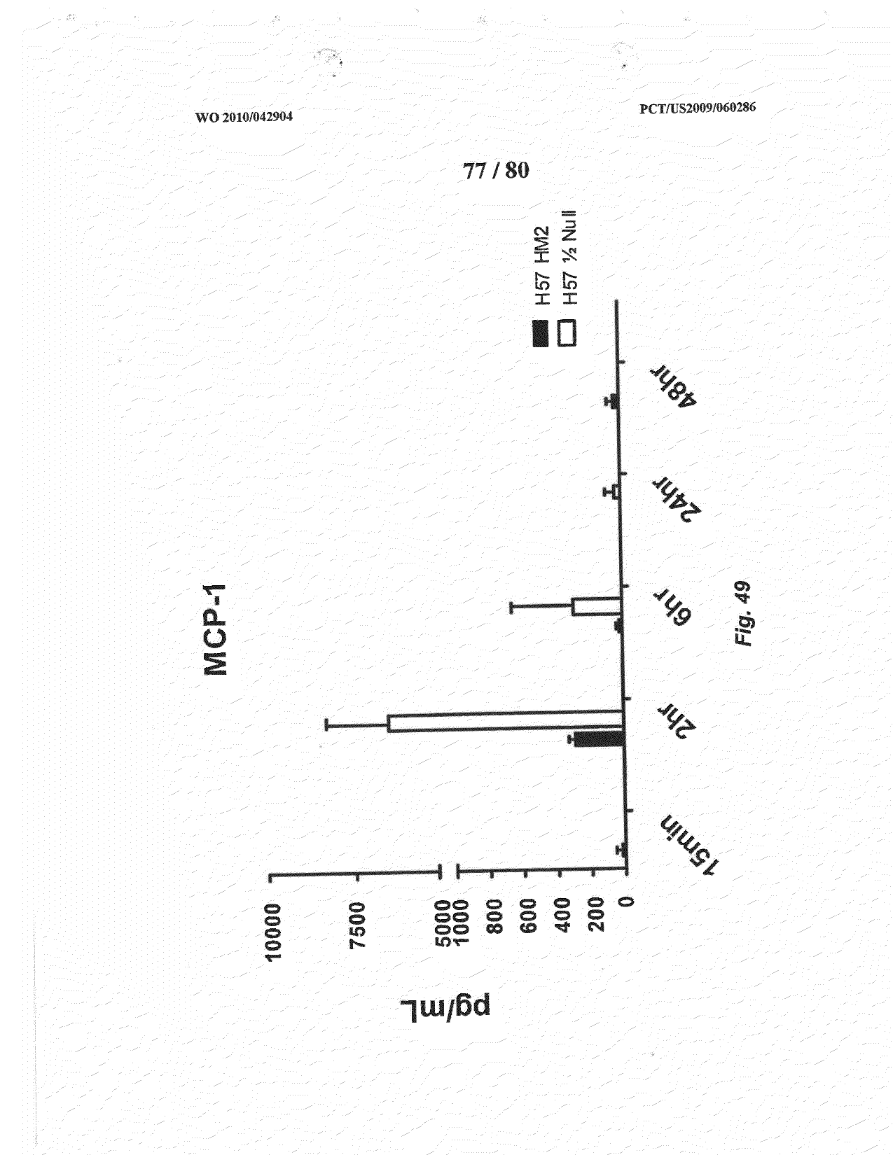

Serum samples were analyzed for the presence of cytokines and chemokines using a custom 14-plex Luminex-based detection kit from Millipore as previously described. Pharmacokinetic disposition parameters for each protein were estimated by non-compartmental analysis using WinNonlin™ Professional software (v5.0.1) and applying the precompiled model 201 for IV bolus administration and sparse sampling. The PK results are provided in FIG. 40 and the calculated half-lives are provided in Table 2 below, while the cytokine results are provided in FIGS. 40-49.

TABLE 2
PK Results

| Test Compound | Serum Half Life (hrs) |
|---|---|
| H57 Null2 (SEQ ID NO: 96) | 83.5 |
| H57 half null (SEQ ID | 40.7 |
| NO: 304) | |
| H57-HM2 (SEQ ID NO: 306) | 6.6 |
| BC3-HM1 (SEQ ID NO: 84) | 3.2 |
| BC3 IgG2-AA-N297A (SEQ | 87.5 |
| ID NO: 82) | |
| BC3IgG4-AA-N297A (SEQ ID | 99.7 |
| NO: 83) | |
| OKT3 IgG2-AA-N297A (SEQ | 42.4 |
| ID NO: 90) | |

The results of the PK study show the SMIP proteins that contain a CH2CH3 tail have a much longer half-life than those that contain CH3 only tails.

FIGS. 39-48 show that the H57-HM2 SMIP protein generally did not cause elevated levels of most cytokines (IFN-γ, IL-2, IL-5, IL-6, or IL-17) at all the time points measured. This may be due in part to the shorter half-life of this molecule. In addition, the few elevated levels of cytokine observed were generally periodic and always lower than the levels seen with the H57 half null SMIP fusion protein.

### Example 14

### In Vitro Studies of Exemplary Fusion Proteins Containing H57Binding Domain

MLR and ConA blast restimulation assays were performed according to the methods in Example 6.

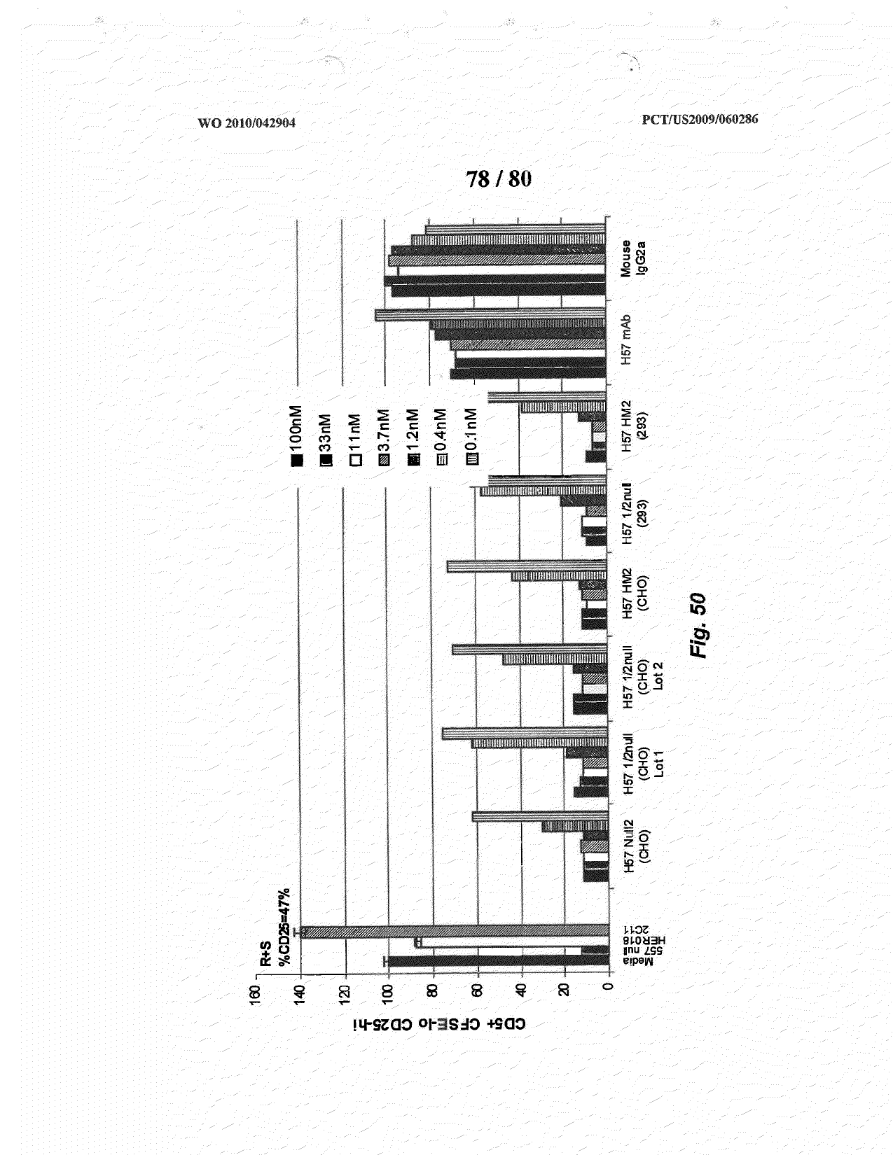

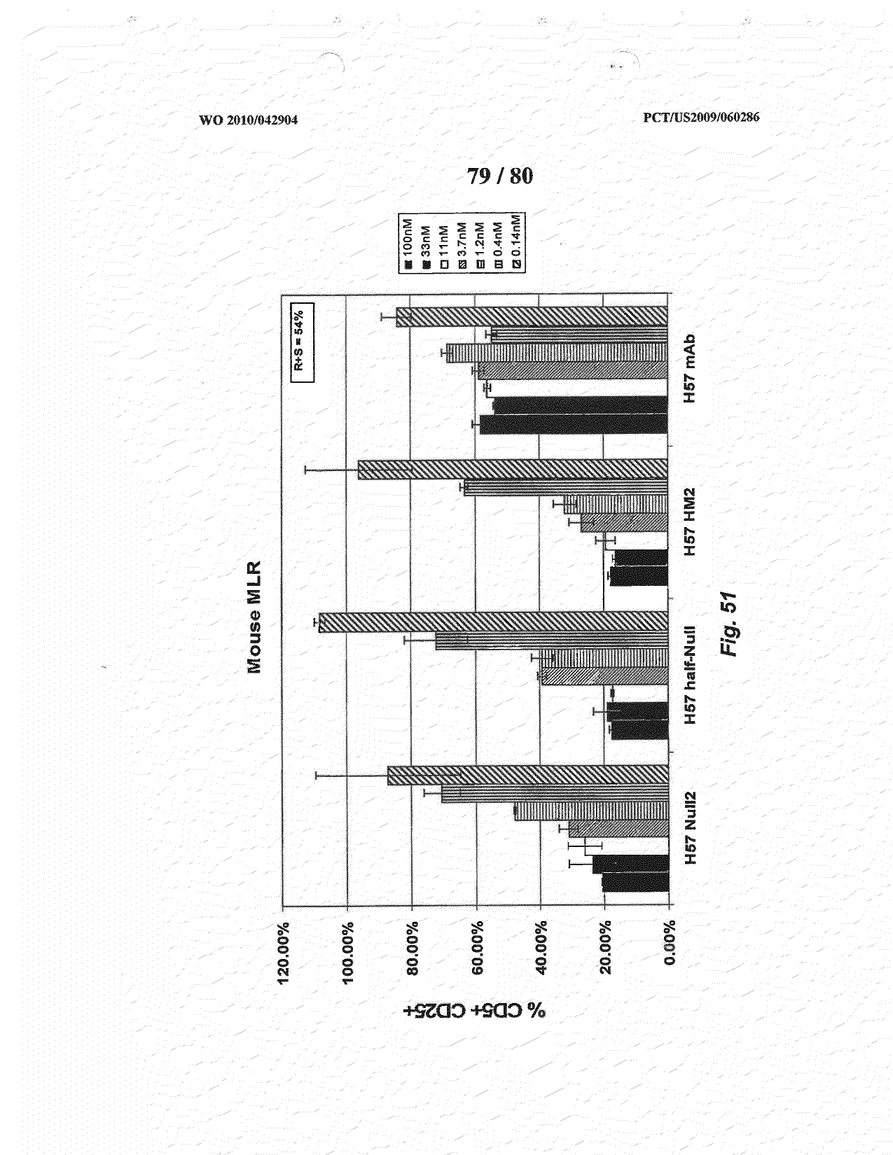

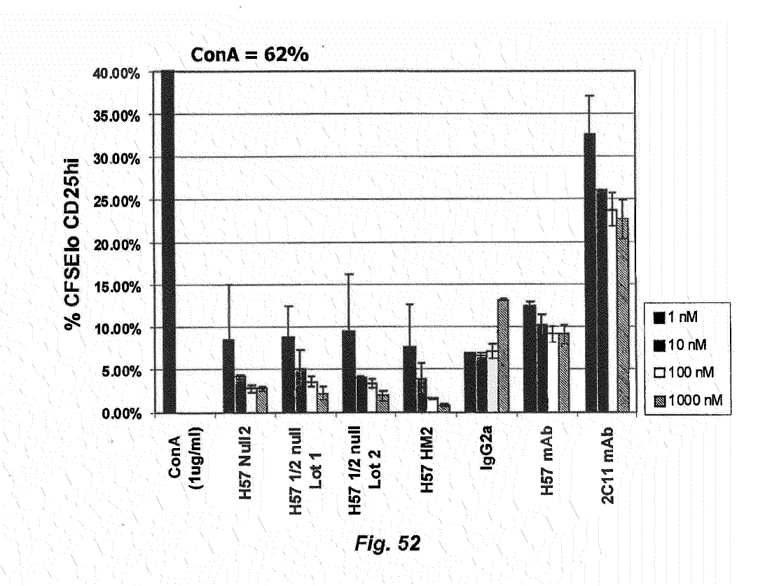

The results show that H57 Null2, H57 half null and H57-HM2 fusion proteins (SEQ ID NOS:96, 304 and 306, respectively), but not H57 mAb blocked primary T cell response to antigen (FIGS. 50 and 51). In addition, H57 Null2, H57 half null and H57-HM2 fusion proteins and IgG2a did not induce activation of ConA-primed T cells, H57 mAb slightly induced activation of ConA-primed T cells, and 2C11 mAb induced activation of ConA-primed T cells (FIG. 52). H57 Null2 and H57-HM2 fusion proteins did not induce cytokine release in ConA blast restimulation assays, while H57 half null fusion protein resulted in higher levels of some cytokines tested (e.g., GM-CSF, IFN-γ, IL-4, IL-5, IL-6, IL-10, IL-17, IP-10 and TNF-α) compared to H57 Null2 and H57-HM2 fusion proteins (data not shown).

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety.

These and other changes can be made to the embodiments in light of the above-detailed description. In general, in the following claims, the terms used should not be construed to limit the claims to specific embodiments disclosed in the specification and the claims, but should be construed to include all possible embodiments along with the full scope of equivalents to which such claims are entitled. Accordingly, the claims are not limited by this disclosure.

MEGA

1. A recombinant binding protein comprising a binding domain that specifically binds to a TCR complex, wherein the binding domain comprises the amino acid sequence as set forth in SEQ ID NO:245 and the amino acid sequence as set forth in SEQ ID NOS:241.

2. The protein of claim 1, wherein the amino acids set forth in SEQ ID NOs:245 and 241 are joined by a linker comprising GlySer, Gly₂Ser (SEQ ID NO:339), Gly₃Ser (SEQ ID NO:340), Gly₄Ser (SEQ ID NO:341), Gly₅Ser (SEQ ID NO:342), (Gly₃Ser)₁(Gly₄Ser)₁ (SEQ ID NO:343), (Gly₃Ser)₂(Gly₄Ser)₁ (SEQ ID NO:344), (Gly₃Ser)₃(Gly₄Ser)₁ (SEQ ID NO:345), (Gly₃Ser)₄(Gly₄Ser)₁ (SEQ ID NO:346), (Gly₃Ser)₅(Gly₄Ser)₁ (SEQ ID NO:347), (Gly₃Ser)₁(Gly₄Ser)₁ (SEQ ID NO:348), (Gly₃Ser)₁(Gly₄Ser)₂ (SEQ ID NO:349), (Gly₃Ser)₁(Gly₄Ser)₃ (SEQ ID NO:350), (Gly₃Ser)₁(Gly₄Ser)₄ (SEQ ID NO:351), (Gly₃Ser)₁(Gly₄Ser)₅ (SEQ ID NO:352), (Gly₃Ser)₃(Gly₄Ser)₃ (SEQ ID NO:353), (Gly₃Ser)₄(Gly₄Ser)₄ (SEQ ID NO:354), (Gly₃Ser)₅(Gly₄Ser)₅ (SEQ ID NO:355), (Gly₄Ser)₂ (SEQ ID NO:356), (Gly₄Ser)₃ (SEQ ID NO:145), (Gly₄Ser)₄ (SEQ ID NO:357), (Gly₄Ser)₅ (SEQ ID NO:358) or GGGGSGGGGSGGGGSAQ (SEQ ID NO:98).

3. The protein of claim 1, wherein the binding domain comprises the amino acid sequence as set forth in SEQ ID NO:263.

4. The protein of claim 1, wherein the protein comprises an immunologlobulin CH2 region polypeptide and an immunologlobulin CH3 region polypeptide.

5. The protein of claim 4, wherein the immunologlobulin CH2 region polypeptide comprises:
(i) an amino acid substitution at the asparagine of position 297 and one or more substitutions or deletions at positions 234 to 238;
(ii) one or more substitutions or deletions at positions 234 to 238 and at least one substitution at position 253, 310, 318, 320, 322, or 331; or
(iii) an amino acid substitution at the asparagine of position 297, one or more substitutions or deletions at positions 234 to 238 and at least one substitution at position 253, 310, 318, 320, 322, or 331.

6. The protein of claim 4, wherein the immunologlobulin CH2 region polypeptide comprises an amino acid substitution at the asparagine of position 297, amino acid substitutions at positions 234, 235 and 237, and an amino acid deletion at position 236.

7. The protein of claim 5, wherein the amino acid substitution at position 297 is an Asn to Ala substitution.

8. The protein of claim 4, wherein the binding domain is linked to the CH2 or CH3 group by an immunoglobulin hinge region polypeptide.

9. The protein of claim 8, wherein the immunoglobulin hinge region polypeptide is selected from the group consisting of a wild type human IgG1 hinge, a human IgG1 hinge with at least one cysteine mutated, a wild type mouse IGHG2c hinge, any one of the amino acid sequences set forth in SEQ ID NOS:212-218, 300 and 379-434, and amino acids 3-17 of SEQ ID NO:10.

10. The protein of claim 4, comprising an immunoglobulin CH2 region polypeptide as set forth in any one of SEQ ID NOS:75, 102-104 and 375-378.

11. The protein of claim 4, wherein:
(i) the immunoglobulin CH2 region polypeptide is a human IgG2 CH2 region polypeptide and the immunoglobulin CH3 region polypeptide is a human IgG2 CH3 region polypeptide; or
(ii) the immunoglobulin CH2 region polypeptide is a human IgG4 CH2 region polypeptide and the immunoglobulin CH3 region polypeptide is a human IgG4 CH3 region polypeptide.

12. The protein of claim 1, wherein the protein does not contain an immunoglobulin CH2 region polypeptide.

13. The protein of claim 1, comprising a sequence as set forth in any one of SEQ ID NOS:290-293.

14. The protein of claim 1, wherein the protein does not induce or induces a minimally detectable cytokine release.

15. The protein of claim 1, wherein the protein does not activate or minimally activates T cells.

16. A composition comprising the protein of claim 1 and a pharmaceutically acceptable carrier, diluent, or excipient.

17. A polynucleotide encoding the protein of claim 1.

18. An expression vector comprising the polynucleotide of claim 17 operably linked to an expression control sequence.

19. A method of reducing rejection of solid organ transplant, comprising administering to a solid organ transplant recipient an effective amount of the protein of claim 1.

20. A method for treating an autoimmune disease, comprising administering to a patient in need thereof an effective amount of the protein of claim 1.

21. The method of claim 20, wherein the autoimmune disease is selected from the group consisting of an inflammatory bowel disease, Crohn's disease, ulcerative colitis, diabetes mellitus, asthma and arthritis.
